(19) United States
(12) Patent Application Publication
(10) Pub. No.: US 2023/0332184 A1
(43) Pub. Date: Oct. 19, 2023

(54) TEMPLATE GUIDE RNA MOLECULES

(71) Applicant: FLAGSHIP PIONEERING INNOVATIONS VI, LLC, Cambridge, MA (US)

(72) Inventors: Jacob Rosenblum RUBENS, Cambridge, MA (US); Robert James CITORIK, Somerville, MA (US); Anne Helen BOTHMER, Cambridge, MA (US); Aamir MIR, Cambridge, MA (US); John Frederick BRIONES, Cambridge, MA (US); William Edward SALOMON, West Roxbury, MA (US)

(21) Appl. No.: 18/061,632

(22) Filed: Dec. 5, 2022

Related U.S. Application Data
(63) Continuation of application No. PCT/US2021/036055, filed on Jun. 5, 2021.
(60) Provisional application No. 63/035,663, filed on Jun. 5, 2020.

(51) Int. Cl. C12N 15/90; C12N 15/11; C12N 9/22; C12N 9/12

(52) U.S. Cl. CPC C12N 15/907; C12N 15/11; C12N 15/111; C12N 9/22; C12N 9/1276; C12N 9/1252; C12N 2310/531; C12N 2310/20; C12N 2800/80; C12N 2320/52; C12N 2310/3231

(57) ABSTRACT

The disclosure is directed, in part, to improved systems and nucleic acids for modifying target DNA.

### RELATED APPLICATIONS

This application is a continuation of International Application No.: PCT/US2021/036055, filed Jun. 5, 2021, which claims priority to U.S. Ser. No. 63/035,663, filed Jun. 5, 2020, the entire contents of which is incorporated herein by reference.

### SEQUENCE LISTING

The instant application contains a Sequence Listing which has been submitted electronically in XML format and is hereby incorporated by reference in its entirety. Said XML copy, created on Jan. 19, 2023, is named V2065-701420_SL.xml and is 79,593 bytes in size.

### BACKGROUND

Integration of a nucleic acid of interest into a genome occurs at low frequency and with little site specificity, in the absence of a specialized protein to promote the insertion event. Some existing approaches, like CRISPR/Cas9, are more suited for small edits that rely on host repair pathways, and are less effective at integrating longer sequences. Other existing approaches, like Cre/loxP, require a first step of inserting a loxP site into the genome and then a second step of inserting a sequence of interest into the loxP site. There is a need in the art for improved compositions (e.g., proteins and nucleic acids) and methods for inserting, altering, or deleting sequences of interest in a genome.

### SUMMARY OF THE INVENTION

This disclosure relates to novel compositions, systems and methods for altering a genome at one or more locations in a host cell, tissue or subject, in vivo or in vitro. In particular, the invention features compositions, systems and methods for inserting, altering, or deleting sequences of interest in a host genome. The invention features, in part, improved template nucleic acids (e.g., template RNAs) that provide improvements to said compositions, systems and methods for inserting, altering, or deleting sequences of interest in a host genome.

Features of the compositions or methods can include one or more of the following enumerated embodiments.

1. A system for modifying DNA comprising:
(a) polypeptide or a nucleic acid encoding the polypeptide, wherein the polypeptide comprises a reverse transcriptase (RT) domain and optionally an endonuclease domain, e.g., nickase domain; and
(b) a template RNA (or DNA encoding the template RNA) comprising (e.g., from 5′ to 3′) (i) optionally a sequence that binds a target site in the DNA (e.g., a second strand of a site in a target genome), (ii) a sequence that binds the polypeptide, (iii) a heterologous object sequence, and (iv) optionally a 3′ target homology domain,
wherein the heterologous object sequence comprises an alteration relative to a corresponding original sequence (e.g., a wild-type sequence), wherein the alteration improves the speed, fidelity, or speed and fidelity of target-primed reverse transcription by the reverse transcriptase.
   2. A system for modifying DNA comprising:
(a) polypeptide or a nucleic acid encoding the polypeptide, wherein the polypeptide comprises a reverse transcriptase (RT) domain and optionally an endonuclease domain, e.g., nickase domain; and
(b) a template RNA (or DNA encoding the template RNA) comprising (e.g., from 5′ to 3′) (i) optionally a sequence that binds a target site in the DNA (e.g., a second strand of a site in a target genome), (ii) a sequence that binds the polypeptide, (iii) a heterologous object sequence, and (iv) optionally a 3′ target homology domain,
wherein the template RNA comprises an alteration relative to a corresponding original sequence (e.g., a wild-type sequence), wherein the alteration improves the speed, fidelity, or speed and fidelity of target-primed reverse transcription by the reverse transcriptase,
wherein optionally the alteration is in (iv) or (i).
   3. The system of embodiment 2, wherein the alteration is in a region of (iv) that has complementarity with a second region in the template RNA, e.g., wherein the second region is a region in (i).
   4. The system of embodiment 2, wherein the alteration is in a region of (i) that has complementarity with a second region in the template RNA, e.g., wherein the second region is a region in (iv).
   5. The system of any of embodiments 1-4, wherein the alteration comprises a chemical modification that destabilizes RNA:RNA pairing relative to a corresponding unmodified nucleotide.
   6. The system of any of embodiments 1-5, wherein the original sequence corresponding to (i) is the sequence of the corresponding target site, e.g., a target site in the human genome.
   7. The system of any of embodiments 1-6, wherein the original sequence corresponding to (iv) is the sequence of the corresponding target site, e.g., a target site in the human genome.
   8. A system for modifying DNA comprising:
(a) polypeptide or a nucleic acid encoding the polypeptide, wherein the polypeptide comprises a reverse transcriptase (RT) domain and optionally an endonuclease domain, e.g., nickase domain; and
(b) a template RNA (or DNA encoding the template RNA) comprising (e.g., from 5′ to 3′) (i) optionally a sequence that binds a target site in the DNA (e.g., a second strand of a site in a target genome), (ii) a sequence that binds the polypeptide, (iii) a heterologous object sequence, and (iv) optionally a 3′ target homology domain,
wherein the heterologous object sequence has one or both of the following characteristics:
i) does not comprise self-complementary sequences, e.g., that form hairpin structures, e.g., under stringent conditions, or if a self-complementary sequence is present, it has one, two, or all of the following characteristics:
   (1) each self-complementary sequence is no more than 10, 9, 8, 7, 6, 5, 4, or 3 nucleotides in length,
   (2) the self-complementary sequence forms a hairpin comprising arms of no longer than 10, 9, 8, 7, 6, 5, 4, or 3 nucleotides in length, or
   (3) the self-complementary sequence comprises at least 1, 2, 3, 4, or 5 positions of non-complementarity (e.g., mismatches or bulges) with its partner sequence,
ii) does not comprise a repetitive sequence (e.g., a single-, di-, or tri-nucleotide repetitive sequence) or if a repetitive sequence is present it is of no more than 12, 11, 10, 9, 8, 7, or 6 nucleotides in length.
   9. The system of any of embodiments 1-8, wherein (a) comprises an endonuclease domain, e.g., a nickase domain.
   10. The system of any of embodiments 1-9, further comprising a second polypeptide or nucleic acid encoding a second polypeptide comprising an endonuclease domain.
   11. The system of any of embodiments 1-10, wherein (a) further comprises a DNA-binding domain.
   12. The system of either of embodiments 10 or 11, wherein the second polypeptide further comprise a DNA-binding domain.
   13. A system for modifying DNA comprising:
(a) first polypeptide or a nucleic acid encoding the first polypeptide, wherein the first polypeptide comprises DNA polymerase activity (e.g., comprises a reverse transcriptase (RT) domain) and optionally comprises an endonuclease domain, e.g., nickase domain;
(b) a first template RNA (or DNA encoding the first template RNA) comprising (e.g., from 5′ to 3′) (i) optionally a sequence that binds a first target site in a DNA (e.g., a second strand of a site in a target genome), (ii) a sequence that binds the polypeptide, (iii) a heterologous object sequence, and (iv) optionally a 3′ target homology domain;
(c) optionally, a second polypeptide or a nucleic acid encoding the second polypeptide, wherein the second polypeptide comprises DNA polymerase activity (e.g., comprises a reverse transcriptase (RT) domain) and optionally comprises an endonuclease domain, e.g., nickase domain; and
(d) a second template RNA (or DNA encoding the second template RNA) comprising (e.g., from 5′ to 3′) (i) optionally a sequence that binds a second target site in the DNA (e.g., a second strand of a site in a target genome), (ii) a sequence that binds the polypeptide (e.g., the polypeptide of (a) or the polypeptide of (c)), (iii) a heterologous object sequence, and (iv) optionally a 3′ target homology domain;
wherein the first target site and the second target site are on antiparallel strands of the DNA;
wherein optionally the second polypeptide has an identical sequence to the first polypeptide;
wherein optionally one or both of the first template RNA and the second template RNA is a template RNA described herein.
   14. The system of embodiment 13, wherein the polypeptide of (a)) comprises a RNA-dependent DNA polymerase activity and optionally a DNA-dependent DNA polymerase activity.
   15. The system of embodiment 13, wherein the polypeptide of (c) comprises a RNA-dependent DNA polymerase activity and optionally a DNA-dependent DNA polymerase activity.
   16. The system of embodiment 13, wherein the heterologous object sequence of the first template RNA comprises a first region that is complementary to a second region of the heterologous object sequence of the second template RNA.
   17. The system of embodiment 16, wherein the first region is situated at the 5′ end of the heterologous object sequence of the first template RNA, and the second region is situated at the 5′ end of the heterologous object sequence of the second template RNA
   18. The system of embodiment 13, wherein the first and second target sites are located about 5, 6, 7, 8, 9, 10, 11, 12, 13, 14, 15, 16, 17, 18, 19, 20, 25, 30, 40, 50, 60, 70, 80, 90, 100, 150, 200, 300, 400, 500, 600, 700, 800, 900, 1000, 1500, 2000, 2500, 3000, 4000, 5000, 6000, 7000, 8000, 9000, or 10,000 nucleotides apart from each other on the DNA.
   19. The system of embodiment 13 or 18, wherein the first and second target sites are located about 5-10, 10-20, 20-30, 30-40, 40-50, 50-60, 60-70, 70-80, 80-90, 90-100, 100-200, 200-300, 300-400, 400-500, 500-600, 600-700, 700-800, 800-900, 900-1000, 1000-2000, 2000-3000, 3000-4000, 4000-5000, 5000-6000, 6000-7000, 7000-8000, 8000-9000, or 9000-10,000 nucleotides apart from each other on the DNA.
   20. The system of any of embodiments 13-19, wherein the first polypeptide has at least 75%, 80%, 85%, 90%, 95%, 96%, 97%, 98%, 99%, or 100% amino acid sequence identity relative to the second polypeptide.
   21. The system of any of embodiments 13-20, wherein the first target site is positioned within about 1, 2, 3, 4, 5, 6, 7, 8, 9, or 10 nucleotides (e.g., adjacent to) a first protospacer adjacent motif (PAM); and/or wherein the second target site is positioned within about 1, 2, 3, 4, 5, 6, 7, 8, 9, or 10 nucleotides (e.g., adjacent to) a second PAM.
   22. The system of embodiment 21, wherein the first PAM and the second PAM share at least 66%, 75%, or 100% sequence identity.
   23. The system of any of embodiments 13-22, wherein the system is capable of introducing deletions and/or insertions into the DNA (e.g., deletions or insertions of at least about 10, 20, 30, 40, 50, 60, 70, 80, 90, 100, 200, 300, 400, 500, 600, 700, 800, 900, or 1000 nucleotides).
   24. A system for modifying DNA comprising:
(a) polypeptide or a nucleic acid encoding the polypeptide, wherein the polypeptide comprises a reverse transcriptase (RT) domain and optionally an endonuclease domain, e.g., nickase domain; and
(b) a template RNA (or DNA encoding the template RNA) comprising (e.g., from 5′ to 3′) (i) optionally a sequence that binds a target site in the DNA (e.g., a second strand of a site in a target genome), (ii) a sequence that binds the polypeptide, (iii) a heterologous object sequence, (iv) optionally a 3′ target homology domain, and (v) a reverse transcriptase termination moiety.
   25. A template RNA comprising (e.g., from 5′ to 3′) (i) optionally a sequence that binds a target site in the DNA (e.g., a second strand of a site in a target genome), (ii) a sequence that binds a polypeptide comprising a reverse transcriptase (RT) domain and/or an endonuclease domain, (iii) a heterologous object sequence, (iv) optionally a 3′ target homology domain, and (v) a reverse transcriptase termination moiety.
   26. The system or template RNA of embodiment 24 or 25, wherein the reverse transcriptase termination moiety is positioned between (ii) the sequence that binds the polypeptide and (iii) the heterologous object sequence.
   27. The system or template RNA of any of embodiments 24-26, wherein the reverse transcriptase termination moiety comprises a streptavidin moiety.
   28. The system or template RNA of embodiment 27, wherein the sequence that binds the polypeptide is attached to a first biotin moiety bound to the streptavidin moiety, and/or wherein the heterologous object sequence is attached to a second biotin moiety bound to the streptavidin moiety.
   29. The system or template RNA of any of embodiments 24-26, wherein the reverse transcriptase termination moiety comprises an artificially stabilized hairpin.
   30. The system or template RNA of any of embodiments 24-26, wherein the reverse transcriptase termination moiety comprises a spacer (e.g., a C3 spacer or a tri/hexa-ethylene glycol spacer).
   31. The system or template RNA of any of embodiments 24-26, wherein the reverse transcriptase termination moiety comprises a trizole moiety (e.g., a trizole moiety produced by click chemistry).
   32. A system for modifying DNA comprising:
(a) polypeptide or a nucleic acid encoding the polypeptide, wherein the polypeptide comprises a reverse transcriptase (RT) domain and optionally an endonuclease domain, e.g., nickase domain; and
(b) a template RNA (or DNA encoding the template RNA) comprising (e.g., from 5′ to 3′) (i) optionally a sequence that binds a target site in the DNA (e.g., a second strand of a site in a target genome), (ii) a sequence that binds the polypeptide, (iii) a heterologous object sequence, (iv) optionally a 3′ target homology domain, and (v) a region capable of hybridizing to either of (i) or (iv), or portions (e.g., having a length of about 5-10 nucleotides) or combinations thereof.
   33. The system of embodiment 32, wherein the region of (v) is positioned at one end of the template nucleic acid.
   34. The system of embodiment 33, wherein the region of (v) is attached to (i), e.g., at the 3′ end of (i).
   35. The system of embodiment 33, wherein the region of (v) is attached to (iv), e.g., at the 5′ end of (iv).
   36. The system of any of embodiments 32-35, wherein the hybridization of the region of (v) to (i) or (iv), or the portion or combination thereof, forms a hairpin.
   37. The system of any of embodiments 32-36, wherein hybridization between (i) and (iv), or portions thereof (e.g., having a length of at least 10, 20, 30, 40, 50, 60, 70, 80, 90, or 100 nucleotides) is reduced, e.g., by at least 50%, 60%, 70%, 75%, 80%, 85%, 90%, 95%, 96%, 97%, 98%, 99%, or 100%, by presence of the region of (v).
   38. The system of any of embodiments 32-37, wherein the region of (v) comprises the reverse complement of the sequence of (i), or a sequence having at least 75%, 80%, 85%, 90%, 95%, 96%, 97%, 98%, or 99% sequence identity thereto.
   39. The system of any of embodiments 32-38, wherein the region of (v) comprises the reverse complement of (i), or a sequence having no more than 1, 2, 3, 4, 5, 6, 7, 8, 9 or 10 mismatches relative thereto.
   40. The system of any of embodiments 32-39, wherein the region of (v) comprises the reverse complement of (i), or a sequence having 1, 2, or 3 mismatches relative thereto.
   41. The system of any of embodiments 32-40, wherein the region of (v) comprises the reverse complement of the sequence of (iv), or a sequence having at least 75%, 80%, 85%, 90%, 95%, 96%, 97%, 98%, or 99% sequence identity thereto.
   42. The system of any of embodiments 32-41, wherein the region of (v) comprises the reverse complement of (iv), or a sequence having no more than 1, 2, 3, 4, 5, 6, 7, 8, 9 or 10 mismatches relative thereto.
   43. The system of any of embodiments 32-42, wherein the region of (v) comprises the reverse complement of (iv), or a sequence having 1, 2, or 3 mismatches relative thereto.
   44. The system of any of embodiments 32-43, wherein the template RNA comprises a ribozyme (e.g., a hammerhead ribozyme or an HDV ribozyme).
   45. The system of embodiment 44, wherein the ribozyme cleaves the region of (v) from the remainder of the template nucleic acid.
   46. The system of any of embodiments 32-45, wherein the template RNA comprises an aptamer.
   47. The system of embodiment 46, wherein the aptamer binds to a ligand (e.g., a small molecule), e.g., theophylline or guanine, or a variant or derivative thereof.
   48. The system of any of embodiments 32-47, wherein the template RNA comprises an aptazyme, wherein the aptazyme comprises an aptamer domain and a self-cleaving ribozyme domain, wherein binding of a ligand to the aptamer domain stimulates activity of the self-cleaving ribozyme domain.
   49. The system of embodiment 48, wherein the aptazyme cleaves the region of (v) from the remainder of the template RNA in the presence of (e.g., when bound to) the ligand (e.g., a small molecule), e.g., theophylline, guanine, or a variant or derivative thereof.
   50. The system of any of embodiments 32-49, wherein the template RNA comprises a photocleavable region (e.g., positioned between the region of (v) and the remainder of the template nucleic acid).
   51. The system of embodiment 50, wherein excitation of the photocleavable region (e.g., by a particular wavelength of light) results in cleavage of the region of (v) from the remainder of the template RNA.
   52. A template RNA (or DNA encoding the template RNA) comprising (e.g., from 5′ to 3′) (i) optionally a sequence that binds a target site (e.g., a second strand of a site in a target genome), (ii) a sequence that binds a polypeptide comprising a reverse transcriptase (RT) or RT domain, (iii) a heterologous object sequence, and (iv) optionally a 3′ target homology domain, wherein the heterologous object sequence comprises an alteration relative to a corresponding original sequence (e.g., a wild-type sequence), wherein the alteration improves the speed, fidelity, or speed and fidelity of target-primed reverse transcription by a reverse transcriptase (RT).
   53. A template RNA (or DNA encoding the template RNA) comprising (e.g., from 5′ to 3′) (i) optionally a sequence that binds a target site (e.g., a second strand of a site in a target genome), (ii) a sequence that binds a polypeptide comprising a reverse transcriptase (RT) or RT domain, (iii) a heterologous object sequence, and (iv) optionally a 3′ target homology domain, wherein the template RNA comprises an alteration relative to a corresponding original sequence (e.g., a wild-type sequence), wherein the alteration improves the speed, fidelity, or speed and fidelity of target-primed reverse transcription by the reverse transcriptase, wherein optionally the alteration is in (iv) or (i).
   54. A template RNA (or DNA encoding the template RNA) comprising (e.g., from 5′ to 3′) (i) optionally a sequence that binds a target site (e.g., a second strand of a site in a target genome), (ii) a sequence that binds a polypeptide comprising a reverse transcriptase (RT) or RT domain, (iii) a heterologous object sequence, and (iv) optionally a 3′ target homology domain,

wherein the heterologous object sequence has one or both of the following characteristics:
i) does not comprise self-complementary sequences, e.g., that form hairpin structures, e.g., under stringent conditions, or if a self-complementary sequence is present, it has one, two, or all of the following characteristics:
   (1) each self-complementary sequence is no more than 10, 9, 8, 7, 6, 5, 4, or 3 nucleotides in length,
   (2) the self-complementary sequence forms a hairpin comprising arms of no longer than 10, 9, 8, 7, 6, 5, 4, or 3 nucleotides in length, or
   (3) the self-complementary sequence comprises at least 1, 2, 3, 4, or 5 positions of non-complementarity (e.g., mismatches or bulges) with its partner sequence,
ii) does not comprise a repetitive sequence (e.g., a single-, di-, or tri-nucleotide repetitive sequence) or if a repetitive sequence is present it is of no more than 12, 11, 10, 9, 8, 7, or 6 nucleotides in length.
   55. The system or template RNA of any preceding embodiment, wherein the template RNA comprises a 3′ target homology domain.
   56. The system or template RNA of any of embodiments 1-12 or 52-55, wherein improving speed comprises increasing the processivity of the polypeptide (e.g., the RT or RT domain), the polymerization rate of the polypeptide (e.g., the RT or RT domain), or both.
   57. The system or template RNA of any of embodiments 1-12 or 52-56, wherein improving fidelity comprises decreasing the error rate of the polypeptide (e.g., the RT or RT domain).
   58. The system or template RNA of any of embodiments 1-12 or 52-57, wherein improving fidelity comprises preventing or decreasing the amount of incorporation of non-heterologous object sequence template RNA sequence into the target site in the DNA (e.g., the target site in a target genome).
   59. The system or template RNA of any of embodiments 1-12 or 52-58, wherein the alteration comprises an RT terminator sequence situated between the heterologous object sequence and either (i) or (ii), wherein at least (i) or (ii) is present in the template RNA.
   60. The system of any of any preceding embodiment, wherein the polypeptide comprises a reverse transcriptase (RT) domain, e.g., as described herein.
   61. A system for modifying DNA comprising:
(a) a polypeptide or a nucleic acid encoding the polypeptide, wherein the polypeptide comprises a reverse transcriptase (RT) domain and optionally an endonuclease domain, e.g., a nickase domain; and
(b) a template RNA (or DNA encoding the template RNA) comprising (e.g., from 5′ to 3′) (i) optionally a sequence that binds a target site (e.g., a second strand of a site in a target genome), (ii) a sequence that binds the polypeptide, (iii) a heterologous object sequence, and (iv) optionally a 3′ target homology domain,
wherein the template RNA further comprises an RT terminator sequence situated between the heterologous object sequence and either (i) or (ii).
   62. A template RNA (or DNA encoding the template RNA) comprising (e.g., from 5′ to 3′) (i) optionally a sequence that binds a target site (e.g., a second strand of a site in a target genome), (ii) a sequence that binds a polypeptide comprising a reverse transcriptase (RT) or RT domain, (iii) a heterologous object sequence, and (iv) optionally a 3′ target homology domain,

wherein the template RNA further comprises an RT terminator sequence situated between the heterologous object sequence and either (i) or (ii).

63. The system or template RNA of any of embodiments 1-60, wherein the template RNA further comprises an RT terminator sequence situated between the heterologous object sequence and either (i) or (ii).
64. The system or template RNA of either of embodiments 61 or 62, wherein the heterologous object sequence comprises an alteration relative to a corresponding original sequence (e.g., a wild-type sequence), wherein the alteration improves the speed, fidelity, or speed and fidelity of target-primed reverse transcription by the reverse transcriptase.
65. The system or template RNA of any preceding embodiment, wherein the heterologous object sequence encodes a polypeptide or portion thereof or comprises a sequence that is the reverse complement of a sequence encoding a polypeptide or portion thereof.
66. The system or template RNA of embodiment 65, wherein the alteration comprises a change to the sequence encoding the polypeptide or portion thereof or to the sequence that is the reverse complement of a sequence encoding a polypeptide or portion thereof.
67. The system or template RNA of any of embodiments 1-7 or 52-66, wherein the corresponding original sequence is a wild-type gene sequence, a wild-type mRNA sequence or reverse complement thereof, an original nucleic acid sequence encoding a mutant protein, original nucleic acid sequence encoding an artificial protein (e.g., fusion protein), or a sequence encoding a protective mutation, or a portion of any thereof.
68. The system or template RNA of any of embodiments 1-7 or 52-67, wherein the template RNA further comprises one or more additional alterations that do not affect speed, fidelity, or both.
69. The system or template RNA of any of embodiments 1-7 or 52-68, wherein the template RNA further comprises one or more additional alterations that improve speed, fidelity, or both.
70. The system or template RNA of any of embodiments 1-7 or 52-69, wherein the alteration comprises one or more substitutions in the nucleic acid sequence of the heterologous object sequence relative to the original sequence.
71. The system or template RNA of any of embodiments 1-7 or 52-670, wherein the heterologous object sequence encodes a polypeptide or portion thereof or comprises a sequence that is the reverse complement of a sequence encoding a polypeptide or portion thereof, and wherein the alteration does not change the amino acid sequence of the polypeptide or portion thereof.
72. The system or template RNA of any of embodiments 1-7 or 52-71, wherein the original sequence comprises a first self-complementary region and a second self-complementary region, and wherein the alteration is in the first self-complementarity region and reduces complementarity between the first self-complementary region and the second self-complementary region.
73. The system or template RNA of embodiment 72, wherein the first self-complementary region and the second self-complementary region of the original sequence are each independently at least 4, 5, 6, 7, 8, 9, 10, 11, 12, 13, 14, 15, 16, 17, 18, 19, or 20 nucleotides long (and optionally no more than 5, 6, 7, 8, 9, 10, 11, 12, 13, 14, 15, 16, 17, 18, 19, 20, or 30 nucleotides long).
74. The system or template RNA of either of embodiments 72 or 73, wherein the first self-complementary region and the second self-complementary region of the original sequence have no more than 1, 2, or 3 positions of non-complementarity (e.g., mismatches or bulges).
75. The system or template RNA of any of embodiments 72-74, wherein the first self-complementary region and the second self-complementary region of the original sequence comprise a region of perfect complementarity 3, 4, 5, 6, 7, 8, 9, or 10 base pairs in length.
76. The system or template RNA of any of embodiments 72-75, wherein the first self-complementary region and the second self-complementary region of the original sequence comprise a region of partial complementarity, wherein the positions of non-complementarity comprise one or more (e.g., all) wobble base pairs (e.g., G to U, hypoxanthine (I) to U, I to A, or I to C).
77. The system or template RNA of any of the preceding embodiments, wherein the alteration disrupts a G to C base pairing of the first self-complementary region and the second self-complementary region.
78. The system or template RNA of any of the preceding embodiments, wherein the alteration disrupts an A to U base pairing of the first self-complementary region and the second self-complementary region.
79. The system or template RNA of any of the preceding embodiments, wherein the alteration disrupts an G to U base pairing of the first self-complementary region and the second self-complementary region.
80. The system or template RNA of any of the preceding embodiments, wherein the alteration changes (e.g., increases) the minimum free energy of folding (e.g., predicted minimum free energy of folding) of the template RNA, e.g., as measured by a tool such as mRNAoptimiser as described in Example 2.
81. The system or template RNA of any of the preceding embodiments, wherein the alteration increases the minimum free energy of folding (e.g., predicted minimum free energy of folding) of the template RNA by at least 1, 2, 3, 4, 5, 6, 7, 8, 9, 10, 15, 20, 25, 30, 35, 40, 45, 50, 55, 60, 65, 70, 75, 80, 85, 90, 95, or 100 kcal/mol, e.g., as measured by a tool such as mRNAoptimiser as described in Example 2.
82. The system or template RNA of any of the preceding embodiments, wherein the alteration increases the minimum free energy of folding (e.g., predicted minimum free energy of folding) of the template RNA by at least 5, 10, 15, 20, 25, 30, 35, 40, 45, 50, 55, 60, 65, 70, 75, 80, 85, 90, 95, or 100% relative to the minimum free energy of folding of an otherwise similar template RNA lacking the alteration, e.g., as measured by a tool such as mRNAoptimiser as described in Example 2.
83. The system or template RNA of any of the preceding embodiments, wherein the heterologous object sequence encodes a polypeptide or portion thereof or comprises a sequence that is the reverse complement of a sequence encoding a polypeptide or portion thereof, and wherein the alteration comprises a change to the wobble position (the third position) of a codon encoding the polypeptide or portion thereof.
84. The system or template RNA of any of the preceding embodiments, wherein the alteration does not change the function of a polypeptide or portion thereof encoded by the heterologous object sequence.
85. The system or template RNA of any of the preceding embodiments, wherein the alteration reduces the predicted secondary structure of the heterologous object sequence, e.g., as predicted by RNAstructure (Bellaousov et al. Nucleic Acids Res 41:W471-W474 (2013)), e.g., increases the folding free energy by at least 10, 20, 30, 40, or 50 kcal/mol.
86. The system or template RNA of any of the preceding embodiments, wherein the alteration eliminates a hairpin structure from the heterologous object sequence.
87. The system or template RNA of any of the preceding embodiments, wherein the alteration decreases the length of a hairpin structure in the heterologous object sequence, e.g., by 1, 2, 3, 4, 5, 6, 7, 8, 9, or 10 nucleotides.
88. The system or template RNA of any of the preceding embodiments, wherein the alteration replaces a G with an A, a G with a T, or a G with a U.
89. The system or template RNA of any of the preceding embodiments, wherein the alteration replaces a C with an A, a C with a T, or a C with a U.
90. The system or template RNA of any of the preceding embodiments, wherein the alteration decreases the GC content level of the heterologous object sequence (e.g., by at least 1, 2, 3, 4, 5, 6, 7, 8, 9, 10, 15, 20, 25, or 30%).
91. The system or template RNA of any of the preceding embodiments, wherein the alteration eliminates or shortens a repetitive sequence in the heterologous object sequence, e.g., a single, di- or tri-nucleotide repeat.
92. The system or template RNA of any of the preceding embodiments, wherein the processivity of a polypeptide, e.g., a polypeptide comprising an RT domain, reverse transcribing the heterologous object sequence is increased relative to the processivity of a polypeptide, e.g., a polypeptide comprising an RT domain, reverse transcribing a sequence similar to the heterologous object sequence but not comprising the alteration.
93. The system or template RNA of any of the preceding embodiments, wherein the processivity of a polypeptide, e.g., a polypeptide comprising an RT domain, reverse transcribing the heterologous object sequence is at least 1, 2, 3, 4, 5, 6, 7, 8, 9, 10, 20, 30, 40, 50, 60, 70, 80, 90, 100, 120, 140, 160, 180, 200, 300, 400, 500, or 1000% increased relative to the processivity of a polypeptide, e.g., a polypeptide comprising an RT domain, reverse transcribing a sequence similar to the heterologous object sequence but not comprising the alteration.
94. The system or template RNA of any of the preceding embodiments, wherein the polymerization rate of a polypeptide, e.g., a polypeptide comprising an RT domain, reverse transcribing the heterologous object sequence is increased relative to the polymerization rate of a polypeptide, e.g., a polypeptide comprising an RT domain, reverse transcribing a sequence similar to the heterologous object sequence but not comprising the alteration.
95. The system or template RNA of any of the preceding embodiments, wherein the polymerization rate of a polypeptide, e.g., a polypeptide comprising an RT domain, reverse transcribing the heterologous object sequence is at least 1, 2, 3, 4, 5, 6, 7, 8, 9, 10, 20, 30, 40, 50, 60, 70, 80, 90, 100, 120, 140, 160, 180, 200, 300, 400, 500, or 1000% increased relative to the polymerization rate of a polypeptide, e.g., a polypeptide comprising an RT domain, reverse transcribing a sequence similar to the heterologous object sequence but not comprising the alteration.
96. The system or template RNA of any of the preceding embodiments, wherein the error rate of a polypeptide, e.g., a polypeptide comprising an RT domain, reverse transcribing the heterologous object sequence is decreased relative to the error rate a polypeptide, e.g., a polypeptide comprising an RT domain, reverse transcribing a sequence similar to the heterologous object sequence but not comprising the alteration (e.g., as measured by Next Generation Sequencing (NGS)).
97. The system or template RNA of any of the preceding embodiments, wherein the error rate of a polypeptide, e.g., a polypeptide comprising an RT domain, reverse transcribing the heterologous object sequence is at least 1, 2, 3, 4, 5, 6, 7, 8, 9, 10, 20, 30, 40, 50, 60, 70, 80, 90, or 100% decreased relative to the error rate of a polypeptide, e.g., a polypeptide comprising an RT domain, reverse transcribing a sequence similar to the heterologous object sequence but not comprising the alteration (e.g., as measured by Next Generation Sequencing (NGS)).
98. The system or template RNA of any of the preceding embodiments, wherein contacting a plurality of cells with the system or a system comprising the template RNA produces a higher number of genomic modifications comprising the heterologous object sequence compared to contacting a similar plurality of cells with an otherwise similar system comprising a template RNA not comprising the alteration.
99. The system or template RNA of any of the preceding embodiments, wherein contacting a plurality of cells with the system or a system comprising the template RNA produces at least 1, 2, 3, 4, 5, 6, 7, 8, 9, 10, 20, 30, 40, 50, 60, 70, 80, 90, 100, 120, 140, 160, 180, 200, 300, 400, 500, or 1000% more genomic modifications comprising the heterologous object sequence compared to contacting a similar plurality of cells with an otherwise similar system comprising a template RNA not comprising the alteration.
100. The system or template RNA of any of the preceding embodiments, wherein contacting a plurality of cells with the system or a system comprising the template RNA produces a higher fraction of complete integrations into genomic DNA (e.g., comprising the entire heterologous object sequence) and optionally a lower fraction of incomplete integrations into genomic DNA (e.g., comprising only a portion of the heterologous object sequence) compared to contacting a similar plurality of cells with an otherwise similar system comprising a template RNA not comprising the alteration.
101. The system or template RNA of any of the preceding embodiments, wherein contacting a plurality of cells with the system or a system comprising the template RNA produces an at least 1, 2, 3, 4, 5, 6, 7, 8, 9, 10, 20, 30, 40, 50, 60, 70, 80, 90, 100, 120, 140, 160, 180, 200, 300, 400, 500, or 1000% higher fraction of complete integrations into genomic DNA (e.g., comprising the entire heterologous object sequence) and optionally an at least 1, 2, 3, 4, 5, 6, 7, 8, 9, 10, 20, 30, 40, 50, 60, 70, 80, 90, 100, 120, 140, 160, 180, 200, 300, 400, 500, or 1000% lower fraction of incomplete integrations into genomic DNA (e.g., comprising only a portion of the heterologous object sequence) compared to contacting a similar plurality of cells with an otherwise similar system comprising a template RNA not comprising the alteration.
102. The system or template RNA of any of the preceding embodiments, wherein the RT terminator sequence is situated 5′ of the heterologous object sequence on the template RNA.
103. The system or template RNA of any of the preceding embodiments, wherein the RT terminator sequence is situated no more than 1, 2, 3, 4, 5, 6, 7, 8, 9, 10, 11, 12, 13, 14, 15, 16, 17, 18, 19, 20, 30, 40, or 50 nucleotides from the heterologous object sequence.
104. The system or template RNA of any of the preceding embodiments, wherein the RT terminator sequence is situated directly adjacent to the heterologous object sequence.
105. The system or template RNA of any of the preceding embodiments, wherein the RT terminator sequence is situated within the heterologous object sequence, e.g., at the 5′ end of the heterologous object sequence, e.g., no more than 1, 2, 3, 4, 5, 6, 7, 8, 9, 10, 11, 12, 13, 14, 15, 16, 17, 18, 19, 20, 30, 40, or 50 nucleotides from the 5′ end of heterologous object sequence.
106. The system or template RNA of any of the preceding embodiments, wherein the RT terminator sequence is not situated in a protein coding sequence of the heterologous object sequence.
107. The system or template RNA of any of the preceding embodiments, wherein the RT terminator sequence is situated 3′ of (i) the sequence that binds a target site, (ii) the sequence that binds the polypeptide comprising an RT domain, or both (i) and (ii).
108. The system or template RNA of any of the preceding embodiments, wherein the RT terminator sequence is situated no more than 1, 2, 3, 4, 5, 6, 7, 8, 9, 10, 11, 12, 13, 14, 15, 16, 17, 18, 19, 20, 30, 40, or 50 nucleotides from the sequence that binds a target site.
109. The system or template RNA of any of the preceding embodiments, wherein the RT terminator sequence is situated directly adjacent to the sequence that binds a target site.
110. The system or template RNA of any of the preceding embodiments, wherein the RT terminator sequence is situated no more than 1, 2, 3, 4, 5, 6, 7, 8, 9, 10, 11, 12, 13, 14, 15, 16, 17, 18, 19, 20, 30, 40, or 50 nucleotides from the sequence that binds the polypeptide, e.g., the polypeptide comprising an RT domain.
111. The system or template RNA of any of the preceding embodiments, wherein the RT terminator sequence is situated directly adjacent to the sequence that binds the polypeptide, e.g., the polypeptide comprising an RT domain.
112. The system or template RNA of any of the preceding embodiments, wherein the RT terminator sequence comprises a sequence (e.g., a termination sequence) from the genome of a virus, e.g., a retrovirus, e.g., HIV.
113. The system or template RNA of any of the preceding embodiments, wherein the RT terminator sequence comprises some or all of the HIV-1 central termination sequence (CTS), e.g., Ter1 and/or Ter2.
114. The system of any of the preceding embodiments, wherein contacting a plurality of cells with the system produces fewer genomic modifications comprising template RNA sequence that is not the heterologous object sequence compared to contacting a similar plurality of cells with a similar system comprising a template RNA not comprising the RT terminator sequence.
115. The system of any of the preceding embodiments, wherein contacting a plurality of cells with the system produces at least 1, 2, 3, 4, 5, 6, 7, 8, 9, 10, 20, 30, 40, 50, 60, 70, 80, 90, or 100% fewer genomic modifications comprising template RNA sequence that is not the heterologous object sequence compared to contacting a similar plurality of cells with a similar system comprising a template RNA not comprising the RT terminator sequence.
116. The system of any of the preceding embodiments, wherein contacting a plurality of cells with the system produces fewer genomic modifications comprising (i) the sequence that binds the target site or (ii) the sequence that binds the polypeptide, e.g., the polypeptide comprising an RT domain, compared to contacting a similar plurality of cells with a similar system comprising a template RNA not comprising the RT terminator sequence.
117. The system of any of the preceding embodiments, wherein contacting a plurality of cells with the system produces at least 1, 2, 3, 4, 5, 6, 7, 8, 9, 10, 20, 30, 40, 50, 60, 70, 80, 90, or 100% fewer genomic modifications comprising (i) the sequence that binds the target site or (ii) the sequence that binds the polypeptide, e.g., the polypeptide comprising an RT domain, compared to contacting a similar plurality of cells with a similar system comprising a template RNA not comprising the RT terminator sequence.
118. The template RNA of any of the preceding embodiments, wherein reverse transcription (RT) of the heterologous object sequence by a polypeptide, e.g., a polypeptide comprising an RT domain, produces fewer DNA products comprising template RNA sequence that is not the heterologous object sequence compared to RT of a similar heterologous object sequence not comprising the RT terminator sequence.
119. The template RNA of any of the preceding embodiments, wherein reverse transcription (RT) of the heterologous object sequence by a polypeptide, e.g., a polypeptide comprising an RT domain, produces at least 1, 2, 3, 4, 5, 6, 7, 8, 9, 10, 20, 30, 40, 50, 60, 70, 80, 90, or 100% fewer DNA products comprising template RNA sequence that is not the heterologous object sequence compared to RT of a similar heterologous object sequence not comprising the RT terminator sequence (e.g., as measured by long-read PCR).
120. The template RNA of any of the preceding embodiments, wherein reverse transcription (RT) of the heterologous object sequence by a polypeptide, e.g., a polypeptide comprising an RT domain, produces fewer DNA products comprising (i) the sequence that binds the target site or (ii) the sequence that binds the polypeptide, e.g., the polypeptide comprising an RT domain, compared to RT of a similar heterologous object sequence not comprising the RT terminator sequence.
121. The template RNA of any of the preceding embodiments, wherein reverse transcription (RT) of the heterologous object sequence by a polypeptide, e.g., a polypeptide comprising an RT domain, produces at least 1, 2, 3, 4, 5, 6, 7, 8, 9, 10, 20, 30, 40, 50, 60, 70, 80, 90, or 100% fewer DNA products comprising (i) the sequence that binds the target site or (ii) the sequence that binds the polypeptide, e.g., the polypeptide comprising an RT domain, compared to RT of a similar heterologous object sequence not comprising the RT terminator sequence.
122. The system of any of the preceding embodiments, wherein the template RNA further comprises a 5′ target homology domain, and wherein reverse transcription (RT) of the heterologous object sequence by a polypeptide, e.g., a polypeptide comprising an RT domain, produces fewer DNA products comprising the 5′ target homology domain compared to RT of a similar heterologous object sequence not comprising the RT terminator sequence.
123. The template RNA of any of the preceding embodiments, wherein the template RNA further comprises a 5′ target homology domain, and wherein reverse transcription (RT) of the heterologous object sequence by a polypeptide, e.g., a polypeptide comprising an RT domain, produces at least 1, 2, 3, 4, 5, 6, 7, 8, 9, 10, 20, 30, 40, 50, 60, 70, 80, 90, or 100% fewer DNA products comprising the 5′ target homology domain compared to RT of a similar heterologous object sequence not comprising the RT terminator sequence.
124. The system or template RNA of any of the preceding embodiments, wherein the RT terminator sequence comprises a first self-complementary region and a second self-complementary region, e.g., a palindromic or partially palindromic sequence.
125. The system or template RNA of embodiment 70, wherein the first self-complementary region of the RT terminator sequence hybridizes to the second self-complementary region of the RT terminator sequence, e.g., under stringent conditions.
126. The system or template RNA of any of the preceding embodiments, wherein the RT terminator sequence adopts a secondary or tertiary structure comprising one or more hairpins, e.g., under stringent conditions, e.g., as measured by a computer tool such as RNAstructure.
127. A reaction mixture comprising:

a cell and the system or template RNA of any preceding embodiment, or DNA encoding the same.

128. A reaction mixture comprising:

a DNA comprising a target site and any system or template RNA of any of embodiments 1-126, or DNA encoding the same.

129. A kit comprising:

the system, template RNA, or reaction mixture of any preceding embodiments, and

instructions for using the system, template RNA, or reaction mixture.

130. The kit of embodiment 129, further comprising one or both of a cell or DNA comprising a target site.
131. A lipid nanoparticle (LNP) comprising the system or template RNA, or DNA encoding the system or template RNA of any of embodiments 1-126.
132. A virus, viral-like particle, or virosome comprising the system or template RNA, or DNA encoding the system or template RNA of any of embodiments 1-126.
133. The virus, viral-like particle, or virosome of embodiment 132, wherein the virus, viral-like particle, or virosome comprises an adeno-associated virus (AAV) capsid protein.
134. A method of manufacturing a template RNA, comprising:

providing a first segment of the template RNA;

providing a second segment of the template RNA; and

covalently linking the first and second segments.

135. The method of embodiment 134, wherein covalently linking the first segment to the second segment comprises using ligation (e.g., splint ligation) or click chemistry.
136. The method of any of embodiments 133-135, wherein the first segment comprises (iii) a heterologous object sequence and (iv) a 3′ target homology domain.
137. The method of any of embodiments 133-136, wherein the second segment comprises (ii) a sequence that binds the RT domain and/or endonuclease domain of a polypeptide described herein, and optionally further comprises (i) the sequence that binds a target site in the DNA.
138. The method of any of embodiments 133-137, which further comprises:

providing a first segment of the template RNA; and

covalently linking the third segment to the second segment.

139. The method of embodiment 138, wherein the third segment comprises (i) the sequence that binds a target site in the DNA.
140. A method for manufacturing a template RNA, comprising:
(a) providing a template RNA of any of embodiments 1-126, and
(b) assaying one or more of:
   (i) the length of the template RNA, e.g., whether the template RNA has a length that is above a reference length or within a reference length range, e.g., whether at least 80, 85, 90, 95, 96, 97, 98, or 99% of the template RNA present is greater than 100, 125, 150, 175, or 200 nucleotides long;
   (ii) the presence, absence, and/or length of a polyA tail on the template RNA, e.g., whether at least 80, 85, 90, 95, 96, 97, 98, or 99% of the template RNA present contains a polyA tail (e.g., a polyA tail that is at least 5, 10, 20, or 30 nucleotides in length);
   (iii) the presence, absence, and/or type of a 5′ cap on the template RNA, e.g., whether at least 80, 85, 90, 95, 96, 97, 98, or 99% of the template RNA present contains a 5′ cap, e.g., whether that cap is a 7-methylguanosine cap, e.g., a O-Me-m7G cap;
   (iv) the presence, absence, and/or type of one or more modified nucleotides (e.g., selected from dihydrouridine, inosine, 7-methylguanosine, 5-methylcytidine (5mC), 5′ Phosphate ribothymidine, 2′-O-methyl ribothymidine, 2′-O-ethyl ribothymidine, 2′-fluoro ribothymidine, C-propynyl-deoxycytidine (pdC), C-5 propynyl-deoxyuridine (pdU), C-5 propynyl-cytidine (pC), C-5 propynyl-uridine (pU), 5-methyl cytidine, 5-methyl uridine, 5-methyl deoxycytidine, 5-methyl deoxyuridine methoxy, 2,6-diaminopurine, 5′-Dimethoxytrityl-N4-ethyl-2′-deoxycytidine, C-5 propynyl-f-cytidine (pfC), C-5 propynyl-f-uridine (pfU), 5-methyl f-cytidine, 5-methyl f-uridine, C-5 propynyl-m-cytidine (pmC), C-5 propynyl-f-uridine (pmU), 5-methyl m-cytidine, 5-methyl m-uridine, LNA (locked nucleic acid), MGB (minor groove binder) pseudouridine (T), 1-N-methylpseudouridine (1-Me-′P), or 5-methoxyuridine (5-MO-U)) in the template RNA, e.g., whether at least 80, 85, 90, 95, 96, 97, 98, or 99% of the template RNA present contains one or more modified nucleotides;
   (v) the stability of the template RNA (e.g., over time and/or under a pre-selected condition), e.g., whether at least 80, 85, 90, 95, 96, 97, 98, or 99% of the template RNA remains intact (e.g., greater than 100, 125, 150, 175, or 200 nucleotides long) after a stability test;
   (vi) the potency of the template RNA in a system for modifying DNA, e.g., whether at least 1% of target sites are modified after a system comprising the template RNA is assayed for potency; or
   (vii) the presence, absence, and/or level of one or more of a pyrogen, virus, fungus, bacterial pathogen, or host cell protein, e.g., whether the template RNA is free or substantially free of pyrogen, virus, fungus, bacterial pathogen, or host cell protein contamination.
      141. A method for manufacturing a system for modifying DNA, comprising:
(a) providing a system for modifying DNA of any of embodiments 1-126, and
(b) assaying one or more of:
   (i) the length of the template RNA, e.g., whether the template RNA has a length that is above a reference length or within a reference length range, e.g., whether at least 80, 85, 90, 95, 96, 97, 98, or 99% of the template RNA present is greater than 100, 125, 150, 175, or 200 nucleotides long;
   (ii) the presence, absence, and/or length of a polyA tail on the template RNA, e.g., whether at least 80, 85, 90, 95, 96, 97, 98, or 99% of the template RNA present contains a polyA tail (e.g., a polyA tail that is at least 5, 10, 20, or 30 nucleotides in length);
   (iii) the presence, absence, and/or type of a 5′ cap on the template RNA, e.g., whether at least 80, 85, 90, 95, 96, 97, 98, or 99% of the template RNA present contains a 5′ cap, e.g., whether that cap is a 7-methylguanosine cap, e.g., a O-Me-m7G cap;
   (iv) the presence, absence, and/or type of one or more modified nucleotides (e.g., selected from pseudouridine, dihydrouridine, inosine, 7-methylguanosine, 1-N-methylpseudouridine (1-Me-Ψ), 5-methoxyuridine (5-MO-U), 5-methylcytidine (5mC), or a locked nucleotide in the template RNA, e.g., whether at least 80, 85, 90, 95, 96, 97, 98, or 99% of the template RNA present contains one or more modified nucleotides;
   (v) the stability of the template RNA (e.g., over time and/or under a pre-selected condition), e.g., whether at least 80, 85, 90, 95, 96, 97, 98, or 99% of the template RNA remains intact (e.g., greater than 100, 125, 150, 175, or 200 nucleotides long) after a stability test;
   (vi) the potency of the template RNA in a system for modifying DNA, e.g., whether at least 1% of target sites are modified after a system comprising the template RNA is assayed for potency;
   (vii) the length of the polypeptide, first polypeptide, or second polypeptide, e.g., whether the polypeptide, first polypeptide, or second polypeptide has a length that is above a reference length or within a reference length range, e.g., whether at least 80, 85, 90, 95, 96, 97, 98, or 99% of the polypeptide, first polypeptide, or second polypeptide present is greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long);
   (viii) the presence, absence, and/or type of post-translational modification on the polypeptide, first polypeptide, or second polypeptide, e.g., whether at least 80, 85, 90, 95, 96, 97, 98, or 99% of the polypeptide, first polypeptide, or second polypeptide contains a selected post-translational modification;
   (ix) the presence, absence, and/or type of one or more artificial, synthetic, or non-canonical amino acids in the polypeptide, first polypeptide, or second polypeptide, e.g., whether at least 80, 85, 90, 95, 96, 97, 98, or 99% of the polypeptide, first polypeptide, or second polypeptide present contains one or more artificial, synthetic, or non-canonical amino acids;
   (x) the stability of the polypeptide, first polypeptide, or second polypeptide (e.g., over time and/or under a pre-selected condition), e.g., whether at least 80, 85, 90, 95, 96, 97, 98, or 99% of the polypeptide, first polypeptide, or second polypeptide remains intact (e.g., greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long)) after a stability test;
   (xi) the potency of the polypeptide, first polypeptide, or second polypeptide in a system for modifying DNA, e.g., whether at least 1% of target sites are modified after a system comprising the polypeptide, first polypeptide, or second polypeptide is assayed for potency; or
   (xii) the presence, absence, and/or level of one or more of a pyrogen, virus, fungus, bacterial pathogen, or host cell protein, e.g., whether the system is free or substantially free of pyrogen, virus, fungus, bacterial pathogen, or host cell protein contamination.
      142. A method for modifying a target site in genomic DNA in a cell, the method comprising:

contacting the cell with a system, template RNA, virus, viral-like particle, or virosome, LNP, or DNA encoding the same of any of embodiments 1-126 or 131-133,

thereby modifying the target site in genomic DNA in a cell.

143. A method for treating a subject having a disease or condition associated with a genetic defect, the method comprising:

administering to the subject a system, template RNA, virus, viral-like particle, or virosome, LNP, or DNA encoding the same of any of embodiments 1-126 or 131-133,

thereby treating the subject having a disease or condition associated with a genetic defect.

144. The method of either of embodiments 142 or 143, wherein the disease or condition associated with a genetic defect is an indication listed in any of Tables 9-12 of International Application PCT/US2021/020948 filed Mar. 4, 2021, and/or wherein the genetic defect is a defect in a gene listed in any of Tables 9-12 therein.
145. The method of either of embodiments 142 or 143, wherein the subject is a human patient.
146. The system, kit, polypeptide, or reaction mixture of any of the preceding embodiments, wherein the system comprises one or more circular RNA molecules (circRNAs).
147. The system, kit, polypeptide, or reaction mixture of embodiment 146, wherein the circRNA encodes the polypeptide (e.g., the polypeptide comprising an RT domain).
148. The system, kit, polypeptide, or reaction mixture of any of embodiments 146-147, wherein circRNA is delivered to a host cell.
149. The system, kit, polypeptide, or reaction mixture of any of the preceding embodiments, wherein the circRNA is capable of being linearized, e.g., in a host cell, e.g., in the nucleus of the host cell.
150. The system, kit, polypeptide, or reaction mixture of any of the preceding embodiments, wherein the circRNA comprises a cleavage site.
151. The system, kit, polypeptide, or reaction mixture of any embodiment 150, wherein the circRNA further comprises a second cleavage site.
152. The system, kit, polypeptide, or reaction mixture of embodiment 150 or 151, wherein the cleavage site can be cleaved by a ribozyme, e.g., a ribozyme comprised in the circRNA (e.g., by autocleavage).
153. The system, kit, polypeptide, or reaction mixture of any of the preceding embodiments, wherein the circRNA comprises a ribozyme sequence.
154. The system, kit, polypeptide, or reaction mixture of embodiment 153, wherein the ribozyme sequence is capable of autocleavage, e.g., in a host cell, e.g., in the nucleus of the host cell.
155. The system, kit, polypeptide, or reaction mixture of any of embodiments 153-154, wherein the ribozyme is an inducible ribozyme.
156. The system, kit, polypeptide, or reaction mixture of any of embodiments 153-155 wherein the ribozyme is a protein-responsive ribozyme, e.g., a ribozyme responsive to a nuclear protein, e.g., a genome-interacting protein, e.g., an epigenetic modifier, e.g., EZH2.
157. The system, kit, polypeptide, or reaction mixture of any of embodiments 153-156, wherein the ribozyme is a nucleic acid-responsive ribozyme.
158. The system, kit, polypeptide, or reaction mixture of embodiment 157, wherein the catalytic activity (e.g., autocatalytic activity) of the ribozyme is activated in the presence of a target nucleic acid molecule (e.g., an RNA molecule, e.g., an mRNA, miRNA, ncRNA, lncRNA, tRNA, snRNA, or mtRNA).
159. The system, kit, polypeptide, or reaction mixture of any of embodiments 153-156, wherein the ribozyme is responsive to a target protein (e.g., an MS2 coat protein).
160. The system, kit, polypeptide, or reaction mixture of embodiment 158, wherein the target protein localized to the cytoplasm or localized to the nucleus (e.g., an epigenetic modifier or a transcription factor).
161. The system, kit, polypeptide, or reaction mixture of any of embodiments 153-157, wherein the ribozyme comprises the ribozyme sequence of a B2 or ALU retrotransposon, or a nucleic acid sequence having at least 85%, 90%, 95%, 96%, 97%, 98%, or 99% sequence identity thereto.
162. The system, kit, polypeptide, or reaction mixture of any of embodiments 153-157, wherein the ribozyme comprises the sequence of a tobacco ringspot virus hammerhead ribozyme, or a nucleic acid sequence having at least 85%, 90%, 95%, 96%, 97%, 98%, or 99% sequence identity thereto.
163. The system, kit, polypeptide, or reaction mixture of any of embodiments 153-157, wherein the ribozyme comprises the sequence of a hepatitis delta virus (HDV) ribozyme, or a nucleic acid sequence having at least 85%, 90%, 95%, 96%, 97%, 98%, or 99% sequence identity thereto.
164. The system, kit, polypeptide, or reaction mixture of any of embodiments 153-163, wherein the ribozyme is activated by a moiety expressed in a target cell or target tissue.
165. The system, kit, polypeptide, or reaction mixture of any of embodiments 153-164, wherein the ribozyme is activated by a moiety expressed in a target subcellular compartment (e.g., a nucleus, nucleolus, cytoplasm, or mitochondria).
166. The system, kit, polypeptide, or reaction mixture of any of the preceding embodiments, wherein the ribozyme is comprised in a circular RNA or a linear RNA.
167. A system comprising a first circular RNA encoding the polypeptide of a system as described herein; and a second circular RNA comprising the template RNA of a system as described herein.
168. The system of any of the preceding embodiments, wherein the template RNA, e.g., the 5′ UTR, comprises a ribozyme which cleaves the template RNA (e.g., in the 5′ UTR).
169. The system of any of the preceding embodiments, wherein the template RNA comprises a ribozyme that is heterologous to (b)(i), (b)(ii), (b)(iii), (b)(iv), or a combination thereof.
170. The system of any of the preceding embodiments, wherein the heterologous ribozyme is capable of cleaving RNA comprising the ribozyme, e.g., 5′ of the ribozyme, 3′ of the ribozyme, or within the ribozyme.

### Definitions

Domain: The term “domain” as used herein refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, an endonuclease domain, a DNA binding domain, a reverse transcription domain; an example of a domain of a nucleic acid is a regulatory domain, such as a transcription factor binding domain.

Exogenous: As used herein, the term exogenous, when used with reference to a biomolecule (such as a nucleic acid sequence or polypeptide) means that the biomolecule was introduced into a host genome, cell or organism by the hand of man. For example, a nucleic acid that is as added into an existing genome, cell, tissue or subject using recombinant DNA techniques or other methods is exogenous to the existing nucleic acid sequence, cell, tissue or subject.

First/Second Strand: As used herein, first strand and second strand, as used to describe the individual DNA strands of target DNA, distinguish the two DNA strands based upon which strand the reverse transcriptase domain initiates polymerization, e.g., based upon where target primed synthesis initiates. The first strand refers to the strand of the target DNA upon which the reverse transcriptase domain initiates polymerization, e.g., where target primed synthesis initiates. The second strand refers to the other strand of the target DNA. First and second strand designations do not describe the target site DNA strands in other respects; for example, in some embodiments the first and second strands are nicked by a polypeptide described herein, but the designations ‘first’ and ‘second’ strand have no bearing on the order in which such nicks occur.

Genomic safe harbor site (GSH site): A genomic safe harbor site is a site in a host genome that is able to accommodate the integration of new genetic material, e.g., such that the inserted genetic element does not cause significant alterations of the host genome posing a risk to the host cell or organism. A GSH site generally meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconservered element; (vi) has low transcriptional activity (i.e. no mRNA+/−25 kb); (vii) is not in copy number variable region; (viii) is in open chromatin; and/or (ix) is unique, with 1 copy in the human genome. Examples of GSH sites in the human genome that meet some or all of these criteria include (i) the adeno-associated virus site 1 (AAVS1), a naturally occurring site of integration of AAV virus on chromosome 19; (ii) the chemokine (C—C motif) receptor 5 (CCR5) gene, a chemokine receptor gene known as an HIV-1 coreceptor; (iii) the human ortholog of the mouse Rosa26 locus; (iv) the rDNA locus. Additional GSH sites are known and described, e.g., in Pellenz et al. epub Aug. 20, 2018 (https://doi.org/10.1101/396390).

Heterologous: The term heterologous, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In another example, a heterologous domain of a polypeptide or nucleic acid sequence (e.g., a DNA binding domain of a polypeptide or nucleic acid encoding a DNA binding domain of a polypeptide) may be disposed relative to other domains or may be a different sequence or from a different source, relative to other domains or portions of a polypeptide or its encoding nucleic acid. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

Inverted Terminal Repeats: The term “inverted terminal repeats” or “ITRs” as used herein refers to AAV viral cis-elements named so because of their symmetry. These elements promote efficient multiplication of an AAV genome. It is hypothesized that the minimal elements for ITR function are a Rep-binding site (RBS; 5′-GCGCGCTCGCTCGCTC-3 ‘ (SEQ ID NO: 1) for AAV2) and a terminal resolution site (TRS; 5’-AGTTGG-3′ for AAV2) plus a variable palindromic sequence allowing for hairpin formation. According to the present invention, an ITR comprises at least these three elements (RBS, TRS and sequences allowing the formation of an hairpin). In addition, in the present invention, the term “ITR” refers to ITRs of known natural AAV serotypes (e.g. ITR of a serotype 1, 2, 3, 4, 5, 6, 7, 8, 9, 10 or 11 AAV), to chimeric ITRs formed by the fusion of ITR elements derived from different serotypes, and to functional variant thereof. By functional variant of an ITR, it is referred to a sequence presenting a sequence identity of at least 80%, 85%, 90%, preferably of at least 95% with a known ITR, allowing multiplication of the sequence that includes said ITR in the presence of Rep proteins.

Mutation or Mutated: The term “mutated” when applied to nucleic acid sequences means that nucleotides in a nucleic acid sequence may be inserted, deleted or changed compared to a reference (e.g., native) nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art.

Nucleic acid molecule: Nucleic acid molecule refers to both RNA and DNA molecules including, without limitation, cDNA, genomic DNA and mRNA, and also includes synthetic nucleic acid molecules, such as those that are chemically synthesized or recombinantly produced, such as RNA templates, as described herein. The nucleic acid molecule can be double-stranded or single-stranded, circular or linear. If single-stranded, the nucleic acid molecule can be the sense strand or the antisense strand. Unless otherwise indicated, and as an example for all sequences described herein under the general format “SEQ. ID NO:,” “nucleic acid comprising SEQ. ID NO:1” refers to a nucleic acid, at least a portion which has either (i) the sequence of SEQ. ID NO:1, or (ii) a sequence complimentary to SEQ. ID NO:1. The choice between the two is dictated by the context in which SEQ. ID NO:1 is used. For instance, if the nucleic acid is used as a probe, the choice between the two is dictated by the requirement that the probe be complimentary to the desired target. Nucleic acid sequences of the present disclosure may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule. Other modifications can include, for example, analogs in which the ribose ring contains a bridging moiety or other structure such as modifications found in “locked” nucleic acids. In various embodiments, the nucleic acids are in operative association with additional genetic elements, such as tissue-specific expression-control sequence(s) (e.g., tissue-specific promoters and tissue-specific microRNA recognition sequences), as well as additional elements, such as inverted repeats (e.g., inverted terminal repeats, such as elements from or derived from viruses, e.g., AAV ITRs) and tandem repeats, inverted repeats/direct repeats (e.g., transposon inverted repeats, e.g., transposon inverted repeats also containing direct repeats, e.g., inverted repeats also containing direct repeats), homology regions (segments with various degrees of homology to a target DNA), UTRs (5′, 3′, or both 5′ and 3′ UTRs), and various combinations of the foregoing. The nucleic acid elements of the systems provided by the invention can be provided in a variety of topologies, including single-stranded, double-stranded, circular, linear, linear with open ends, linear with closed ends, and particular versions of these, such as doggybone DNA (dbDNA), close-ended DNA (ceDNA).

Gene expression unit: a gene expression unit is a nucleic acid sequence comprising at least one regulatory nucleic acid sequence operably linked to at least one effector sequence. A first nucleic acid sequence is operably linked with a second nucleic acid sequence when the first nucleic acid sequence is placed in a functional relationship with the second nucleic acid sequence. For instance, a promoter or enhancer is operably linked to a coding sequence if the promoter or enhancer affects the transcription or expression of the coding sequence. Operably linked DNA sequences may be contiguous or non-contiguous. Where necessary to join two protein-coding regions, operably linked sequences may be in the same reading frame.

Host: The terms host genome or host cell, as used herein, refer to a cell and/or its genome into which protein and/or genetic material has been introduced. It should be understood that such terms are intended to refer not only to the particular subject cell and/or genome, but to the progeny of such a cell and/or the genome of the progeny of such a cell. Because certain modifications may occur in succeeding generations due to either mutation or environmental influences, such progeny may not, in fact, be identical to the parent cell, but are still included within the scope of the term “host cell” as used herein. A host genome or host cell may be an isolated cell or cell line grown in culture, or genomic material isolated from such a cell or cell line, or may be a host cell or host genome which composing living tissue or an organism. In some instances, a host cell may be an animal cell or a plant cell, e.g., as described herein. In certain instances, a host cell may be a bovine cell, horse cell, pig cell, goat cell, sheep cell, chicken cell, or turkey cell. In certain instances, a host cell may be a corn cell, soy cell, wheat cell, or rice cell.

Operative association: As used herein, “operative association” describes a functional relationship between two nucleic acid sequences, such as a 1) promoter and 2) a heterologous object sequence, and means, in such example, the promoter and heterologous object sequence (e.g., a gene of interest) are oriented such that, under suitable conditions, the promoter drives expression of the heterologous object sequence. For instance, the template nucleic acid may be single-stranded, e.g., either the (+) or (−) orientation but an operative association between promoter and heterologous object sequence means whether or not the template nucleic acid will transcribe in a particular state, when it is in the suitable state (e.g., is in the (+) orientation, in the presence of required catalytic factors, and NTPs, etc.), it does accurately transcribe. Operative association applies analogously to other pairs of nucleic acids, including other tissue-specific expression control sequences (such as enhancers, repressors and microRNA recognition sequences), IR/DR, ITRs, UTRs, or homology regions and heterologous object sequences or sequences encoding a transposase.

Pseudoknot: A “pseudoknot sequence” sequence, as used herein, refers to a nucleic acid (e.g., RNA) having a sequence with suitable self-complementarity to form a pseudoknot structure, e.g., having: a first segment, a second segment between the first segment and a third segment, wherein the third segment is complementary to the first segment, and a fourth segment, wherein the fourth segment is complementary to the second segment. The pseudoknot may optionally have additional secondary structure, e.g., a stem loop disposed in the second segment, a stem-loop disposed between the second segment and third segment, sequence before the first segment, or sequence after the fourth segment. The pseudoknot may have additional sequence between the first and second segments, between the second and third segments, or between the third and fourth segments. In some embodiments, the segments are arranged, from 5′ to 3′: first, second, third, and fourth. In some embodiments, the first and third segments comprise five base pairs of perfect complementarity. In some embodiments, the second and fourth segments comprise 10 base pairs, optionally with one or more (e.g., two) bulges. In some embodiments, the second segment comprises one or more unpaired nucleotides, e.g., forming a loop. In some embodiments, the third segment comprises one or more unpaired nucleotides, e.g., forming a loop.

Stem-loop sequence: As used herein, a “stem-loop sequence” refers to a nucleic acid sequence (e.g., RNA sequence) with sufficient self-complementarity to form a stem-loop, e.g., having a stem comprising at least two (e.g., 3, 4, 5, 6, 7, 8, 9, or 10) base pairs, and a loop with at least three (e.g., four) base pairs. The stem may comprise mismatches or bulges.

Tissue-specific expression-control sequence(s): As used herein, a “tissue-specific expression-control sequence” means nucleic acid elements that increase or decrease the level of a transcript comprising the heterologous object sequence in the target tissue in a tissue-specific manner, e.g., preferentially in an on-target tissue(s), relative to an off-target tissue(s). In some embodiments, a tissue-specific expression-control sequence preferentially drives or represses transcription, activity, or the half-life of a transcript comprising the heterologous object sequence in the target tissue in a tissue-specific manner, e.g., preferentially in an on-target tissue(s), relative to an off-target tissue(s). Exemplary tissue-specific expression-control sequences include tissue-specific promoters, repressors, enhancers, or combinations thereof, as well as tissue-specific microRNA recognition sequences. Tissue specificity refers to on-target (tissue(s) where expression or activity of the template nucleic acid is desired or tolerable) and off-target (tissue(s) where expression or activity of the template nucleic acid is not desired or is not tolerable). For example, a tissue-specific promoter (such as a promoter in a template nucleic acid or controlling expression of a transposase) drives expression preferentially in on-target tissues, relative to off-target tissues. In contrast, a micro-RNA that binds the tissue-specific microRNA recognition sequences (either on a nucleic acid encoding the transposase or on the template nucleic acid, or both) is preferentially expressed in off-target tissues, relative to on-target tissues, thereby reducing expression of a template nucleic acid (or transposase) in off-target tissues. Accordingly, a promoter and a microRNA recognition sequence that are specific for the same tissue, such as the target tissue, have contrasting functions (promote and repress, respectively, with concordant expression levels, i.e., high levels of the microRNA in off-target tissues and low levels in on-target tissues, while promoters drive high expression in on-target tissues and low expression in off-target tissues) with regard to the transcription, activity, or half-life of an associated sequence in that tissue.

### BRIEF DESCRIPTION OF THE DRAWINGS

FIG. 1 shows the predicted structural characteristics (using RNAstructure) of an exemplary template nucleic acid (e.g., without improvement) as predicted by RNA structure. Arrows in the figure point to the ends of the heterologous object sequence, and nucleotide positions for portions of the template nucleic acid are as follows: (1) gRNA spacer, 1-20; (2) gRNA scaffold, 21-96; (3) Heterologous object sequence, 97-209; (4) 3′ target region, 210-221. FIG. 1 discloses SEQ ID NO: 57.

FIG. 2 shows the predicted structural characteristics of the exemplary template nucleic acid from FIG. 1 with improvements comprising a redesigned heterologous object sequence (mRNAOptimiser). Arrows in the figure point to the ends of the heterologous object sequence, and nucleotide positions for portions of the template nucleic acid are as described in FIG. 1. FIG. 2 discloses SEQ ID NO: 58.

FIG. 3 shows a schematic of the structure of exemplary template RNAs.

FIG. 4 shows a diagram showing the modules of an exemplary template RNA. Individual modules of the exemplary template can be combined, re-arranged, and/or omitted, e.g., to produce a template as described herein. A=5′ homology arm; B=Ribozyme; C=5′ UTR; D=heterologous object sequence; E=3′ UTR; F=3′ homology arm.

FIG. 5 shows a table listing the modules of an exemplary RNA template. Individual modules can be combined, re-arranged, and/or omitted, e.g., to produce a template. A=5′ homology arm; B=Ribozyme; C=5′ UTR; D=heterologous object sequence; E=3′ UTR; F=3′ homology arm.

FIG. 6 is a diagram showing an exemplary dual template system. The system includes two template RNA molecules, each comprising, in order, a priming region, a template region, a reverse transcriptase binding sequence, and a spacer sequence. Each of the template RNAs is bound to a reverse transcriptase (RT) protein (e.g., two identical RT proteins or two different RT proteins). The two template RNAs are bound to the same target DNA, with one template RNA bound to one strand upstream of one PAM, and the other template RNA bound to the antiparallel strand upstream of a second PAM. The two template RNA-RT protein complexes are arranged to move along the target DNA toward each other.

FIGS. 7A-7B are a series of diagrams showing exemplary strategies for preventing reverse transcriptase (RT) read-through into the tracrRNA sequence. The left panel shows a template RNA comprising a generic RT block. The right panel shows a template RNA comprising a biotin-streptavidin RT block, in which the priming and template regions are conjugated to a first biotin moiety, and the spacer region of the template RNA conjugated to a second biotin moiety, with both biotin moieties then bound to a single streptavidin moiety.

FIGS. 8A-8B are a series of diagrams showing exemplary template RNAs each comprising a secondary structural element capable of annealing to one end of the template RNA, thereby reducing or preventing annealing between the spacer region and the priming region of the template RNA. The left panel shows a template RNA in which the secondary structural element is located at the end of the spacer region and is self-annealed to at least a portion of the spacer region. The right panel shows a template RNA in which the secondary structural element is located at the end of the priming region and is self-annealed to at least a portion of the priming region.

FIG. 9 is a diagram showing exemplary self-annealing secondary structural elements at the ends of template RNAs. The top panel shows a secondary structural element having a sequence complementary to the priming region of the template RNA, such that the resultant hairpin formed by its hybridization to the priming region contains no mismatches. The bottom panel shows a secondary structural element having a sequence having at least one mismatch relative to the priming region of the template RNA, such that the resultant hairpin formed by its hybridization to the priming region is destabilized compared to the fully complementary hairpin.

FIG. 10 is a diagram showing an exemplary self-annealing secondary structural element at the end of a template RNA, in which the secondary structural element further comprises a ligand-activated self-cleaving ribozyme (e.g., an aptazyme). When the aptazyme binds to a particular ligand, a cleavage event is triggered that cleaves the self-annealing region of the secondary structural element off from the remainder of the template RNA.

FIG. 11A depicts exemplary positions for chemical modifications in a template RNA. The template RNA comprises (from 5′ to 3′) (i) a sequence that binds a target site in the DNA, (ii) a sequence that binds the polypeptide comprising an RT domain, (iii) a heterologous object sequence (labeled “template”), and (iv) a 3′ target homology domain (labeled “priming”). A Template RNA may comprise chemical modifications at one or more of the positions shown.

FIG. 11B depicts exemplary patterns of chemical modification in a template RNA, e.g., in (i) the sequence that binds a target site in the DNA and (ii) the sequence that binds the polypeptide comprising an RT domain. Top left diagram (SEQ ID NO: 59), see Finn et al, doi.org/10.1016/j.celrep.2018.02.014. Bottom left diagram (SEQ ID NO: 60), see Yin et al, doi.org/10.1038/nbt.4005. Right diagram (SEQ ID NOS 61-62, respectively, in order of appearance), see Mir et al, doi.org/10.1038/s41467-018-05073-z.

FIG. 11C depicts exemplary patterns of chemical modification in a template RNA, e.g., in (iii) the heterologous object sequence and (iv) the 3′ target homology domain (labeled “priming”). In some embodiments, (iii) comprises a region (labeled “nick to edit”) that extends from the end of (iv) to the position corresponding to the location of the nick in the target DNA. In some embodiments, (iii) comprises a region (labeled “edit”) corresponding to the sequence desired to be inserted into the target DNA. In some embodiments, (iii) comprises a region (labeled “RT homology”) that has homology (e.g., identity) to the corresponding region of the target DNA. Unmodified nucleotides are shown in white, and modified nucleotides are shown in gray.

FIG. 11D depicts exemplary patterns of chemical modification in a template RNA, e.g., in (iii) the heterologous object sequence and (iv) the 3′ target homology domain.

FIG. 12 depicts a template RNA and corresponding protein, with oligonucleotides that pair with specific regions of the template RNA and disrupt pairing within the template RNA.

FIG. 13 depicts methods of making a template RNA. Different segments of the template RNA can be synthesized (e.g., by solid phase synthesis) and then assembled, e.g., by click chemistry or splint ligation.

FIG. 14 depicts a circular template RNA.

FIG. 15 depicts a splint oligonucleotide for making a circular template RNA.

### DETAILED DESCRIPTION

This disclosure relates to compositions, systems and methods for targeting, editing, modifying or manipulating a DNA sequence (e.g., inserting a heterologous object sequence into a target site of a mammalian genome) at one or more locations in a DNA sequence in a cell, tissue or subject, e.g., in vivo or in vitro. The heterologous object sequence may include, e.g., a substitution, a deletion, an insertion, e.g., a coding sequence, a regulatory sequence, or a gene expression unit.

More specifically, the disclosure provides reverse transcriptase-based systems for altering a genomic DNA sequence of interest, e.g., by inserting, deleting, or substituting one or more nucleotides into/from the sequence of interest.

The disclosure provides, in part, systems comprising a polypeptide component and a template nucleic acid (e.g., template RNA) component. The disclosure also provides, in part, template nucleic acids (e.g., template RNAs), e.g., for use in a system (e.g., a genome editor system) for modifying DNA. In some embodiments, a genome editor can be used to introduce an alteration into a target site in a genome. In some embodiments, the template nucleic acid (e.g., template RNA) comprises a sequence that binds a target site in the genome (e.g., that binds to a second strand of the target site), a sequence that binds the polypeptide component, a heterologous object sequence, and a 3′ target homology domain. In some embodiments, the polypeptide component comprises a writing domain (e.g., a reverse transcriptase domain). In some embodiments, the polypeptide component comprises a writing domain (e.g., a reverse transcriptase domain) and a DNA-binding domain. In some embodiments, the polypeptide component comprises a writing domain (e.g., a reverse transcriptase domain) and an endonuclease domain (e.g., nickase domain). In some embodiments, the polypeptide component comprises a writing domain (e.g., a reverse transcriptase domain), a DNA-binding domain, and an endonuclease domain (e.g., a nickase domain). Without wishing to be bound by theory, in some embodiments it is thought that the template nucleic acid (e.g., template RNA) binds to the target site in the genome, and binds to the polypeptide component (e.g., localizing the polypeptide component to the target site in the genome). It is thought that the writing domain (e.g., reverse transcriptase domain) of the polypeptide component uses the heterologous object sequence as a template to, e.g., polymerize a sequence complementary to the heterologous object sequence. Without wishing to be bound by theory, it is thought that selection of an appropriate heterologous object sequence can result in substitution, deletion, or insertion of one or more nucleotides at the target site. It is thought that improving the design of the template nucleic acid (e.g., template RNA), e.g., improving the design of the heterologous object sequence and/or proximal sequences, may result in enhanced modification of a target site in the genome.

### Reverse Transcriptase-Based Genome Editors

Genome editors are systems that are capable of modifying a host cell's genome and can be applied for the mutation, deletion, or other modification of a genomic target sequence, including the insertion of heterologous payloads. In some embodiments, these systems take inspiration from a group of naturally evolved mobile genetic elements known as retrotransposons. Genome editor polypeptides can also comprise RT domains derived from sources other than retrotransposons, e.g., from viruses.

Examples of polypeptides (e.g., polypeptide comprising RT domains, e.g., a genome editing polypeptide as described herein), writing domains, DNA-binding domains, endonuclease domains (e.g., nickase domains), and methods of using, combining, and modifying the same can be found, for example, in PCT Publication WO2020/047124, which is hereby incorporated by reference in its entirety. In particular, exemplary RT domains, writing domains, DNA-binding domains, endonuclease domains (e.g., nickase domains), transposons relating to the same, amino acid sequences of any thereof, or nucleic acids encoding any thereof can be found in Tables 1-3 of WO2020/047124, which are hereby incorporated by reference. Exemplary polypeptides comprising an RT domain (e.g., a genome editing polypeptide as described herein) and RT domain sequences are also described, e.g., in U.S. Provisional Application No. 63/035,627 filed Jun. 5, 2020, e.g., at Table 1, Table 3, Table 30, and Table 31 therein; the entire application is incorporated by reference herein including said sequences and tables. Accordingly, a polypeptide comprising an RT domain (e.g., a genome editing polypeptide as described herein) described herein may comprise an amino acid sequence according to any of the Tables mentioned in this paragraph, or a domain thereof (e.g., an RT domain, a DNA binding domain, an RNA binding domain, or an endonuclease domain), or a functional fragment or variant of any of the foregoing, or an amino acid sequence having at least 70%, 80%, 85%, 90%, 95%, or 99% identity thereto.

### Template Nucleic Acid Component of Gene Editor System

The systems described herein can modify a host target DNA site using a template nucleic acid sequence. The systems described herein can transcribe an RNA sequence template into host target DNA sites by target-primed reverse transcription. By writing DNA sequence(s) via reverse transcription of the RNA sequence template directly into the host genome, the system can insert an object sequence into a target genome without the need for exogenous DNA sequences to be introduced into the host cell (unlike, for example, CRISPR systems), as well as eliminate an exogenous DNA insertion step. The system can also delete a sequence from the target genome or introduce a substitution using an object sequence. Therefore, the system provides a platform for the use of customized RNA sequence templates containing object sequences, e.g., sequences comprising heterologous gene coding and/or function information.

The template RNA may have some homology to the target DNA. In some embodiments the template RNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 175, 200 or more bases of exact homology to the target DNA at the 3′ end of the RNA. In some embodiments the template RNA has at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 175, 180, or 200 or more bases of at least 50%, 60%, 70%, 80%, 85%, 90%, 95%, 97%, 98%, 99% or 100% homology to the target DNA, e.g., at the 5′ end of the template RNA. In some embodiments the template RNA has a 3′ untranslated region derived from a non-LTR retrotransposon, e.g. a non-LTR retrotransposons described herein. In some embodiments the template RNA has a 3′ region of at least 10, 15, 20, 25, 30, 40, 50, 60, 80, 100, 120, 140, 160, 180, 200 or more bases of at least 50%, 60%, 70%, 80%, 85%, 90%, 95%, 97%, 98%, 99% or 100% homology to the 3′ sequence of a non-LTR retrotransposon, e.g., a non-LTR retrotransposon described. In some embodiments the template RNA has a 5′ untranslated region derived from a non-LTR retrotransposon, e.g. a non-LTR retrotransposons described herein. In some embodiments the template RNA has a 5′ region of at least 10, 15, 20, 25, 30, 40, 50, 60, 80, 100, 120, 140, 160, 180, or 200 or more bases of at least 40%, 50%, 60%, 70%, 80%, 90%, 95% or greater homology to the 5′ sequence of a non-LTR retrotransposon, e.g., a non-LTR retrotransposon described.

The template RNA component of a genome editing system described herein typically is able to bind the genome editing protein of the system. In some embodiments the template RNA has a 3′ region that is capable of binding a genome editing protein. The binding region, e.g., 3′ region, may be a structured RNA region, e.g., having at least 1, 2 or 3 hairpin loops, capable of binding the genome editing protein of the system.

The template RNA component of a genome editing system described herein typically is able to bind the genome editing protein of the system. In some embodiments the template RNA has a 5′ region that is capable of binding a genome editing protein. The binding region, e.g., 5′ region, may be a structured RNA region, e.g., having at least 1, 2 or 3 hairpin loops, capable of binding the genome editing protein of the system. In some embodiments, the 5′ untranslated region comprises a pseudoknot, e.g., a pseudoknot that is capable of binding to the protein.

In some embodiments, the template RNA (e.g., an untranslated region of the hairpin RNA, e.g., a 5′ untranslated region) comprises a stem-loop sequence. In some embodiments, the template RNA (e.g., an untranslated region of the hairpin RNA, e.g., a 5′ untranslated region) comprises a hairpin. In some embodiments, the template RNA (e.g., an untranslated region of the hairpin RNA, e.g., a 5′ untranslated region) comprises a helix. In some embodiments, the template RNA (e.g., an untranslated region of the hairpin RNA, e.g., a 5′ untranslated region) comprises a pseudoknot. In some embodiments the template RNA comprises a ribozyme. In some embodiments the ribozyme is similar to an hepatitis delta virus (HDV) ribozyme, e.g., has a secondary structure like that of the HDV ribozyme and/or has one or more activities of the HDV ribozyme, e.g., a self-cleavage activity. See, e.g., Eickbush et al., Molecular and Cellular Biology, 2010, 3142-3150.

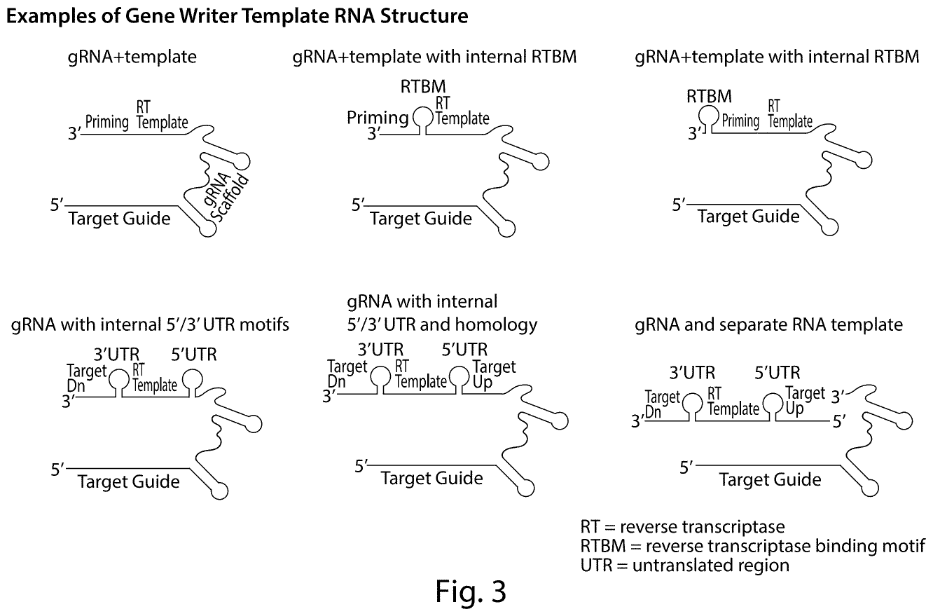

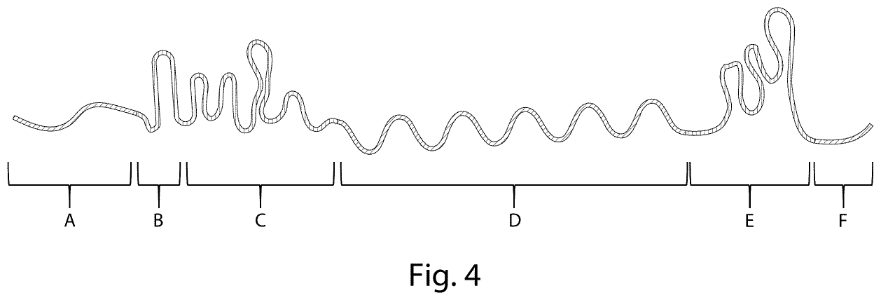

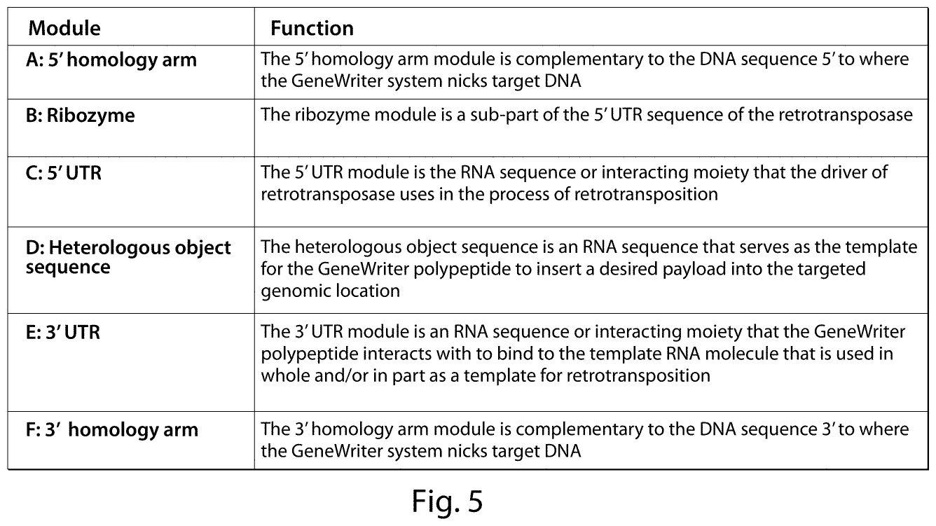

In some embodiments, the template RNA (e.g., an untranslated region of the hairpin RNA, e.g., a 3′ untranslated region) comprises one or more stem-loops or helices. Exemplary structures of R2 3′ UTRs are shown, for example, in Ruschak et al. “Secondary structure models of the 3′ untranslated regions of diverse R2 RNAs” RNA. 2004 June; 10(6): 978-987, e.g., at FIG. 3, therein, and in Eickbush and Eickbush, “R2 and R2/R1 hybrid non-autonomous retrotransposons derived by internal deletions of full-length elements” Mobile DNA (2012) 3:10; e.g., at FIG. 3 therein, which articles are hereby incorporated by reference in their entirety.

In some embodiments, a template RNA described herein comprises a sequence that is capable of binding to a polypeptide (e.g., a polypeptide comprising an RT domain, e.g., a genome editing polypeptide) described herein. For instance, in some embodiments, the template RNA comprises an MS2 RNA sequence capable of binding to an MS2 coat protein sequence in the polypeptide. In some embodiments, the template RNA comprises an RNA sequence capable of binding to a B-box sequence. In some embodiments, the template RNA comprises an RNA sequence (e.g., a crRNA sequence and/or tracrRNA sequence) capable of binding to a dCas sequence in the polypeptide. In some embodiments, in addition to or in place of a UTR, the template RNA is linked (e.g., covalently) to a non-RNA UTR, e.g., a protein or small molecule.

In some embodiments the template RNA has a poly-A tail at the 3′ end. In some embodiments the template RNA does not have a poly-A tail at the 3′ end.

In some embodiments, a template nucleic acid (e.g., template RNA) comprises a 3′ target homology domain. In some embodiments, a 3′ target homology domain is disposed 3′ of the heterologous object sequence and is complementary to a sequence adjacent to a site to be modified by a system described herein, or comprises no more than 1, 2, 3, 4, or 5 mismatches to a sequence complementary to the sequence adjacent to a site to be modified by the system. In some embodiments, the 3′ target homology domain anneals to the target site, which provides a binding site and the 3′ hydroxyl for the initiation of TPRT by a polypeptide (e.g., a polypeptide comprising an RT domain, e.g., a genome editing polypeptide as described herein). In some embodiments, the 3′ target homology domain is 3-5, 5-10, 10-30, 10-25, 10-20, 10-19, 10-18, 10-17, 10-16, 10-15, 10-14, 10-13, 10-12, 10-11, 11-30, 11-25, 11-20, 11-19, 11-18, 11-17, 11-16, 11-15, 11-14, 11-13, 11-12, 12-30, 12-25, 12-20, 12-19, 12-18, 12-17, 12-16, 12-15, 12-14, 12-13, 13-30, 13-25, 13-20, 13-19, 13-18, 13-17, 13-16, 13-15, 13-14, 14-30, 14-25, 14-20, 14-19, 14-18, 14-17, 14-16, 14-15, 15-30, 15-25, 15-20, 15-19, 15-18, 15-17, 15-16, 16-30, 16-25, 16-20, 16-19, 16-18, 16-17, 17-30, 17-25, 17-20, 17-19, 17-18, 18-30, 18-25, 18-20, 18-19, 19-30, 19-25, 19-20, 20-30, 20-25, or 25-30 nt in length, e.g., 10-17, 12-16, or 12-14 nt in length. In some embodiments, the 3′ target homology domain comprises DNA. In some embodiments, the 3′ target homology domain comprises RNA. In some embodiments, the 3′ target homology domain comprises a mixture of DNA and RNA bases.

In some embodiments, a template nucleic acid (e.g., template RNA) comprises a heterologous object sequence. In some embodiments, the heterologous object sequence may be transcribed by the RT domain of a polypeptide (e.g., a polypeptide comprising an RT domain, e.g., a genome editing polypeptide as described herein), e.g., thereby introducing an alteration into a target site in genomic DNA. In some embodiments, the heterologous object sequence is at least 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, or 1,000 nucleotides (nts) in length, or at least 1, 1.5, 2, 2.5, 3, 3.5, 4, 4.5, 5, 5.5, 6, 6.5, 7, 7.5, 8, 8.5, 9, 9.5, or 10 kilobases in length. In some embodiments, the heterologous object sequence is no more than 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 500, 1,000, or 2000 nucleotides (nts) in length, or no more than 20, 15, 10, 9, 8, 7, 6, 5, 4, or 3 kilobases in length. In some embodiments, the heterologous object sequence is 30-1000, 40-1000, 50-1000, 60-1000, 70-1000, 74-1000, 75-1000, 76-1000, 77-1000, 78-1000, 79-1000, 80-1000, 85-1000, 90-1000, 100-1000, 120-1000, 140-1000, 160-1000, 180-1000, 200-1000, 500-1000, 30-500, 40-500, 50-500, 60-500, 70-500, 74-500, 75-500, 76-500, 77-500, 78-500, 79-500, 80-500, 85-500, 90-500, 100-500, 120-500, 140-500, 160-500, 180-500, 200-500, 30-200, 40-200, 50-200, 60-200, 70-200, 74-200, 75-200, 76-200, 77-200, 78-200, 79-200, 80-200, 85-200, 90-200, 100-200, 120-200, 140-200, 160-200, 180-200, 30-100, 40-100, 50-100, 60-100, 70-100, 74-100, 75-100, 76-100, 77-100, 78-100, 79-100, 80-100, 85-100, or 90-100 nucleotides (nts) in length, or 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 5-20, 5-15, 5-10, 5-9, 5-8, 5-7, 5-6, 6-20, 6-15, 6-10, 6-9, 6-8, 6-7, 7-20, 7-15, 7-10, 7-9, 7-8, 8-20, 8-15, 8-10, 8-9, 9-20, 9-15, 9-10, 10-15, 10-20, or 15-20 kilobases in length. In some embodiments, the heterologous object sequence is 10-100, 10-90, 10-80, 10-70, 10-60, 10-50, 10-40, 10-30, or 10-20 nt in length, e.g., 10-80, 10-50, or 10-20 nt in length, e.g., about 10-20 nt in length. In some embodiments, the heterologous object sequence comprises DNA. In some embodiments, the heterologous object sequence comprises RNA. In some embodiments, the heterologous object sequence comprises a mixture of DNA and RNA bases.

In some embodiments, the components and/or functionalities of a template nucleic acid (e.g., template RNA) described herein may be situated in a single template nucleic acid (e.g., template RNA). In some embodiments, a system or method described herein comprises a single template nucleic acid (e.g., template RNA). In some embodiments, the components and/or functionalities of a template nucleic acid (e.g., template RNA) described herein may be disposed amongst a plurality of template nucleic acids (e.g., template RNAs, template RNA and gRNA). In some embodiments a system or method described herein comprises a plurality of template nucleic acids (e.g., template RNAs). For example, a system described herein comprises a first RNA comprising (e.g., from 5′ to 3′) a sequence that binds the polypeptide (e.g., the DNA-binding domain and/or the endonuclease domain, e.g., a gRNA) and a sequence that binds a target site (e.g., a second strand of a site in a target genome), and a second RNA (e.g., a template RNA) comprising (e.g., from 5′ to 3′) optionally a sequence that binds the polypeptide (e.g., that specifically binds the RT domain), a heterologous object sequence, and a 3′ target homology domain. In some embodiments, when the system comprises a plurality of nucleic acids, each nucleic acid comprises a conjugating domain. In some embodiments, a conjugating domain enables association of nucleic acid molecules, e.g., by hybridization of complementary sequences. For example, in some embodiments a first RNA comprises a first conjugating domain and a second RNA comprises a second conjugating domain, and the first and second conjugating domains are capable of hybridizing to one another, e.g., under stringent conditions. In some embodiments, the stringent conditions for hybridization include hybridization in 4× sodium chloride/sodium citrate (SSC), at about 65° C., followed by a wash in 1×SSC, at about 65° C.

In some embodiments, the object sequence may contain an open reading frame. In some embodiments the template nucleic acid (e.g., template RNA) has a Kozak sequence. In some embodiments the template RNA has an internal ribosome entry site. In some embodiments the template RNA has a self-cleaving peptide such as a T2A or P2A site. In some embodiments the template RNA has a start codon. In some embodiments the template RNA has a splice acceptor site. In some embodiments the template RNA has a splice donor site. Exemplary splice acceptor and splice donor sites are described in WO2016044416, incorporated herein by reference in its entirety. Exemplary splice acceptor site sequences are known to those of skill in the art and include, by way of example only, CTGACCCTTCTCTCTCTCCCCCAGAG (SEQ ID NO: 54) (from human HBB gene) and TTTCTCTCCCACAAG (SEQ ID NO: 55) (from human immunoglobulin-gamma gene). In some embodiments the template RNA has a microRNA binding site downstream of the stop codon. In some embodiments the template RNA has a polyA tail downstream of the stop codon of an open reading frame. In some embodiments the template RNA comprises one or more exons. In some embodiments the template RNA comprises one or more introns. In some embodiments the template RNA comprises a eukaryotic transcriptional terminator. In some embodiments the template RNA comprises an enhanced translation element or a translation enhancing element. In some embodiments the RNA comprises the human T-cell leukemia virus (HTLV-1) R region. In some embodiments the RNA comprises a posttranscriptional regulatory element that enhances nuclear export, such as that of Hepatitis B Virus (HPRE) or Woodchuck Hepatitis Virus (WPRE).

In some embodiments, a nucleic acid described herein (e.g., a template RNA or a DNA encoding a template RNA) comprises a microRNA binding site. In some embodiments, the microRNA binding site is used to increase the target-cell specificity of a system, e.g., as described herein. For instance, the microRNA binding site can be chosen on the basis that is is recognized by a miRNA that is present in a non-target cell type, but that is not present (or is present at a reduced level relative to the non-target cell) in a target cell type. Thus, when the template RNA is present in a non-target cell, it would be bound by the miRNA, and when the template RNA is present in a target cell, it would not be bound by the miRNA (or bound but at reduced levels relative to the non-target cell). While not wishing to be bound by theory, binding of the miRNA to the template RNA may interfere with its activity, e.g., may interfere with insertion of the heterologous object sequence into the genome. Accordingly, the system would edit the genome of target cells more efficiently than it edits the genome of non-target cells, e.g., the heterologous object sequence would be inserted into the genome of target cells more efficiently than into the genome of non-target cells, or an insertion or deletion is produced more efficiently in target cells than in non-target cells. A system having a microRNA binding site in the template RNA (or DNA encoding it) may also be used in combination with a nucleic acid encoding a polypeptide (e.g., a polypeptide comprising an RT domain, e.g., a genome editing polypeptide as described herein), wherein expression of the polypeptide is regulated by a second microRNA binding site, e.g., as described herein, e.g., in the section entitled “Polypeptide component of gene editor system”. In some embodiments, e.g., for liver indications, a miRNA is selected from Table 4 of WO2020014209, incorporated herein by reference.

In some embodiments, the object sequence may contain a non-coding sequence. For example, the template nucleic acid (e.g., template RNA) may comprise a regulatory element, e.g., a promoter or enhancer sequence or miRNA binding site. In some embodiments, integration of the object sequence at a target site will result in upregulation of an endogenous gene. In some embodiments, integration of the object sequence at a target site will result in downregulation of an endogenous gene. In some embodiments the template nucleic acid (e.g., template RNA) comprises a tissue specific promoter or enhancer, each of which may be unidirectional or bidirectional. In some embodiments the promoter is an RNA polymerase I promoter, RNA polymerase II promoter, or RNA polymerase III promoter. In some embodiments the promoter comprises a TATA element. In some embodiments the promoter comprises a B recognition element. In some embodiments the promoter has one or more binding sites for transcription factors.

In some embodiments, a nucleic acid described herein (e.g., a template RNA or a DNA encoding a template RNA) comprises a promoter sequence, e.g., a tissue specific promoter sequence. In some embodiments, the tissue-specific promoter is used to increase the target-cell specificity of a system as described herein. For instance, the promoter can be chosen on the basis that it is active in a target cell type but not active in (or active at a lower level in) a non-target cell type. Thus, even if the promoter integrated into the genome of a non-target cell, it would not drive expression (or only drive low level expression) of an integrated gene. A system having a tissue-specific promoter sequence in the template RNA may also be used in combination with a microRNA binding site, e.g., in the template RNA or a nucleic acid encoding a polypeptide (e.g., a polypeptide comprising an RT domain, e.g., a genome editing polypeptide as described herein), e.g., as described herein. A system having a tissue-specific promoter sequence in the template RNA may also be used in combination with a DNA encoding a polypeptide (e.g., a polypeptide comprising an RT domain, e.g., a genome editing polypeptide as described herein), driven by a tissue-specific promoter, e.g., to achieve higher levels of polypeptide in target cells than in non-target cells. In some embodiments, e.g., for liver indications, a tissue-specific promoter is selected from Table 3 of WO2020014209, incorporated herein by reference.

In some embodiments, a system, e.g., DNA encoding a polypeptide as described herein, DNA encoding a template RNA, or DNA or RNA encoding a heterologous object sequence, is designed such that one or more elements is operably linked to a tissue-specific promoter, e.g., a promoter that is active in T-cells. In further embodiments, the T-cell active promoter is inactive in other cell types, e.g., B-cells, NK cells. In some embodiments, the T-cell active promoter is derived from a promoter for a gene encoding a component of the T-cell receptor, e.g., TRAC, TRBC, TRGC, TRDC. In some embodiments, the T-cell active promoter is derived from a promoter for a gene encoding a component of a T-cell-specific cluster of differentiation protein, e.g., CD3, e.g., CD3D, CD3E, CD3G, CD3Z. In some embodiments, T-cell-specific promoters in systems are discovered by comparing publicly available gene expression data across cell types and selecting promoters from the genes with enhanced expression in T-cells. In some embodiments, promoters may be selecting depending on the desired expression breadth, e.g., promoters that are active in T-cells only, promoters that are active in NK cells only, promoters that are active in both T-cells and NK cells.

In some embodiments the template RNA comprises a microRNA sequence, a siRNA sequence, a guide RNA sequence, a piwi RNA sequence.

In some embodiments the template nucleic acid (e.g., template RNA) comprises a site that coordinates epigenetic modification. In some embodiments the template nucleic acid (e.g., template RNA) comprises a chromatin insulator. For example, the template nucleic acid (e.g., template RNA) comprises a CTCF site or a site targeted for DNA methylation.

In some embodiments the template nucleic acid (e.g., template RNA) comprises a gene expression unit composed of at least one regulatory region operably linked to an effector sequence. The effector sequence may be a sequence that is transcribed into RNA (e.g., a coding sequence or a non-coding sequence such as a sequence encoding a microRNA).

In some embodiments the object sequence of the template nucleic acid (e.g., template RNA) is inserted into a target genome in an endogenous intron. In some embodiments the object sequence of the template nucleic acid (e.g., template RNA) is inserted into a target genome and thereby acts as a new exon. In some embodiments the insertion of the object sequence into the target genome results in replacement of a natural exon or the skipping of a natural exon.

In some embodiments, the object sequence of the template nucleic acid (e.g., template RNA) is inserted into the target genome in a genomic safe harbor site, such as AAVS1, CCR5, ROSA26, or albumin locus. In some embodiments, a polypeptide comprising an RT domain (e.g., a genome editing polypeptide as described herein) is used to integrate a CAR into the T-cell receptor a constant (TRAC) locus (Eyquem et al Nature 543, 113-117 (2017)). In some embodiments, a polypeptide comprising an RT domain (e.g., a genome editing polypeptide as described herein) is used to integrate a CAR into a T-cell receptor (3 constant (TRBC) locus. Many other safe harbors have been identified by computational approaches (Pellenz et al Hum Gen Ther 30, 814-828 (2019)) and could be used for RT-mediated integration. In some embodiments, the object sequence of the template nucleic acid (e.g., template RNA) is added to the genome in an intergenic or intragenic region. In some embodiments, the object sequence of the template nucleic acid (e.g., template RNA) is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments, the object sequence of the template nucleic acid (e.g., template RNA) is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments, the object sequence of the template nucleic acid (e.g., template RNA) can be, e.g., 50-50,000 base pairs (e.g., between 50-40,000 bp, between 500-30,000 bp between 500-20,000 bp, between 100-15,000 bp, between 500-10,000 bp, between 50-10,000 bp, between 50-5,000 bp.

The template nucleic acid (e.g., template RNA) can be designed to result in insertions, mutations, or deletions at the target DNA locus. In some embodiments, the template nucleic acid (e.g., template RNA) may be designed to cause an insertion in the target DNA. For example, the template nucleic acid (e.g., template RNA) may contain a heterologous sequence, wherein the reverse transcription will result in insertion of the heterologous sequence into the target DNA. In other embodiments, the RNA template may be designed to write a deletion into the target DNA. For example, the template nucleic acid (e.g., template RNA) may match the target DNA upstream and downstream of the desired deletion, wherein the reverse transcription will result in the copying of the upstream and downstream sequences from the template nucleic acid (e.g., template RNA) without the intervening sequence, e.g., causing deletion of the intervening sequence. In other embodiments, the template nucleic acid (e.g., template RNA) may be designed to write an edit into the target DNA. For example, the template RNA may match the target DNA sequence with the exception of one or more nucleotides, wherein the reverse transcription will result in the copying of these edits into the target DNA, e.g., resulting in mutations, e.g., transition or transversion mutations.

In some embodiments, the template possesses one or more sequences aiding in association of the template with the polypeptide. In some embodiments, these sequences may be derived from retrotransposon UTRs. In some embodiments, the UTRs may be located flanking the desired insertion sequence. In some embodiments, a sequence with target site homology may be located outside of one or both UTRs. In some embodiments, the sequence with target site homology can anneal to the target sequence to prime reverse transcription. In some embodiments, the 5′ and/or 3′ UTR may be located terminal to the target site homology sequence, e.g., such that target primed reverse transcription excludes reverse transcription of the 5′ and/or 3′ UTR. In some embodiments, the system may result in the insertion of a desired payload without any additional sequence (e.g. gene expression unit without UTRs used to bind the polypeptide).

Alternative orientations of the template RNA motifs can be employed, e.g., to limit target site integration to the desired genetic payload. In some embodiments, the polypeptide association domains may be located 5′ of the desired template sequence. For example, the heterologous object sequence may be located downstream of the 5′ UTR and 3′ UTR, giving the 5′-3′ orientation 5′UTR-3′UTR-(heterologous object sequence). In other embodiments, only the 3′ UTR is added upstream of the heterologous object sequence. For example, giving the 5′-3′ orientation 3′UTR-(heterologous object sequence). In certain embodiments, the polypeptide coding region and the heterologous object sequence may be encoded on the same molecule, but where the 5′ UTR (e.g., 5′ UTR from R2 retrotransposon) occurs between the two regions, e.g., giving the 5′-3′ orientation (polypeptide coding sequence)-5′UTR-(heterologous object sequence).

In some embodiments, the template nucleic acid, e.g., template RNA, may comprise a gRNA (e.g., pegRNA). In some embodiments, the template nucleic acid, e.g., template RNA, may bind to the polypeptide by interaction of a gRNA portion of the template nucleic acid with a template nucleic acid binding domain, e.g., a RNA binding domain (e.g., a heterologous RNA binding domain). In some embodiments, the heterologous RNA binding domain is a CRISPR/Cas protein, e.g., Cas9.

In some embodiments, the region of the template nucleic acid, e.g., template RNA, comprising the gRNA adopts an underwound ribbon-like structure of gRNA bound to target DNA (e.g., as described in Mulepati et al. Science 19 Sep. 2014:Vol. 345, Issue 6203, pp. 1479-1484). Without wishing to be bound by theory, this non-canonical structure is thought to be facilitated by rotation of every sixth nucleotide out of the RNA-DNA hybrid. Thus, in some embodiments, the region of the template nucleic acid, e.g., template RNA, comprising the gRNA may tolerate increased mismatching with the target site at some interval, e.g., every sixth base. In some embodiments, the region of the template nucleic acid, e.g., template RNA, comprising the gRNA comprising homology to the target site may possess wobble positions at a regular interval, e.g., every sixth base, that do not need to base pair with the target site.

In some embodiments, a template nucleic acid, e.g., template RNA, comprises a gRNA with inducible activity. Inducible activity may be achieved by the template nucleic acid, e.g., template RNA, further comprising (in addition to the gRNA) a blocking domain, wherein the sequence of a portion of or all of the blocking domain is at least partially complementary to a portion or all of the gRNA. The blocking domain is thus capable of hybridizing or substantially hybridizing to a portion of or all of the gRNA. In some embodiments, the blocking domain and inducibly active gRNA are disposed on the template nucleic acid, e.g., template RNA, such that the gRNA can adopt a first conformation where the blocking domain is hybridized or substantially hybridized to the gRNA, and a second conformation where the blocking domain is not hybridized or not substantially hybridized to the gRNA. In some embodiments, in the first conformation the gRNA is unable to bind to the polypeptide (e.g., the template nucleic acid binding domain, DNA binding domain, or endonuclease domain (e.g., a CRISPR/Cas protein)) or binds with substantially decreased affinity compared to an otherwise similar template RNA lacking the blocking domain. In some embodiments, in the second conformation the gRNA is able to bind to the polypeptide (e.g., the template nucleic acid binding domain, DNA binding domain, or endonuclease domain (e.g., a CRISPR/Cas protein)). In some embodiments, whether the gRNA is in the first or second conformation can influence whether the DNA binding or endonuclease activities of the polypeptide (e.g., of the CRISPR/Cas protein the polypeptide comprises) are active. In some embodiments, hybridization of the gRNA to the blocking domain can be disrupted using an opener molecule. In some embodiments, an opener molecule comprises an agent that binds to a portion or all of the gRNA or blocking domain and inhibits hybridization of the gRNA to the blocking domain. In some embodiments, the opener molecule comprises a nucleic acid, e.g., comprising a sequence that is partially or wholly complementary to the gRNA, blocking domain, or both. By choosing or designing an appropriate opener molecule, providing the opener molecule can promote a change in the conformation of the gRNA such that it can associate with a CRISPR/Cas protein and provide the associated functions of the CRISPR/Cas protein (e.g., DNA binding and/or endonuclease activity). Without wishing to be bound by theory, providing the opener molecule at a selected time and/or location may allow for spatial and temporal control of the activity of the gRNA, CRISPR/Cas protein, or a system comprising the same. In some embodiments, the opener molecule is exogenous to the cell comprising the polypeptide and or template nucleic acid. In some embodiments, the opener molecule comprises an endogenous agent (e.g., endogenous to the cell comprising the polypeptide and or template nucleic acid comprising the gRNA and blocking domain). For example, an inducible gRNA, blocking domain, and opener molecule may be chosen such that the opener molecule is an endogenous agent expressed in a target cell or tissue, e.g., thereby ensuring activity of a system in the target cell or tissue. As a further example, an inducible gRNA, blocking domain, and opener molecule may be chosen such that the opener molecule is absent or not substantially expressed in one or more non-target cells or tissues, e.g., thereby ensuring that activity of a system does not occur or substantially occur in the one or more non-target cells or tissues, or occurs at a reduced level compared to a target cell or tissue. Exemplary blocking domains, opener molecules, and uses thereof are described in PCT App. Publication WO2020044039A1, which is incorporated herein by reference in its entirety.

In some embodiments, the template nucleic acid, e.g., template RNA, may comprise one or more UTRs (e.g. from an R2-type retrotransposon) and a gRNA. In some embodiments, the UTR facilitates interaction of the template nucleic acid (e.g., template RNA) with the writing domain, e.g., reverse transcriptase domain, of the polypeptide. In some embodiments, the gRNA facilitates interaction with the template nucleic acid binding domain (e.g., RNA binding domain) of the polypeptide. In some embodiments, the gRNA directs the polypeptide to the matching target sequence, e.g., in a target cell genome. In some embodiments, the template nucleic acid may contain only the reverse transcriptase binding motif (e.g. 3′ UTR from R2) and the gRNA may be provided as a second nucleic acid molecule (e.g., second RNA molecule) for target site recognition. In some embodiments, the template nucleic acid containing the RT-binding motif may exist on the same molecule as the gRNA, but be processed into two RNA molecules by cleavage activity (e.g. ribozyme).

In some embodiments, a template RNA may be customized to correct a given mutation in the genomic DNA of a target cell (e.g., ex vivo or in vivo, e.g., in a target tissue or organ, e.g., in a subject). For example, the mutation may be a disease-associated mutation relative to the wild-type sequence. Without wishing to be bound by theory, sets of empirical parameters help ensure optimal initial in silico designs of template RNAs or portions thereof. As a non-limiting illustrative example, for a selected mutation, the following design parameters may be employed. In some embodiments, design is initiated by acquiring approximately 500 bp (e.g., up to 50, 100, 150, 200, 250, 300, 350, 400, 450, 500, 550, 600, 650, or 700 bp, and optionally at least 20, 30, 40, 50, 100, 150, 200, 250, 300, 350, 400, 450, 500, 550, 600, or 650 bp) flanking sequence on either side of the mutation to serve as the target region. In some embodiments, a template nucleic acid comprises a gRNA. Methodology for designing gRNAs is known to those of skill in the art. In some embodiments, a gRNA comprises a sequence (e.g., a CRISPR spacer) that binds a target site. In some embodiments, the sequence (e.g., a CRISPR spacer) that binds a target site for use in targeting a template nucleic acid to a target region is selected by considering the particular polypeptide (e.g., endonuclease domain or writing domain, e.g., comprising a CRISPR/Cas domain) being used (e.g., for Cas9, a protospacer-adjacent motif (PAM) of NGG immediately 3′ of a 20 nt gRNA binding region). In some embodiments, the CRISPR spacer is selected by ranking first by whether the PAM will be disrupted by the edit. In some embodiments, disruption of the PAM may increase edit efficiency. In some embodiments, the PAM can be disrupted by also introducing (e.g., as part of or in addition to another modification to a target site in genomic DNA) a silent mutation (e.g., a mutation that does not alter an amino acid residue encoded by the target nucleic acid sequence, if any) in the target site during editing. In some embodiments, the CRISPR spacer is selected by ranking sequences by the proximity of their corresponding genomic site to the desired edit location. In some embodiments, the gRNA comprises a gRNA scaffold. In some embodiments, the gRNA scaffold used may be a standard scaffold (e.g., for Cas9, 5′-GTTTTAGAGCTAGAAATAGCAAGTTAAAATAAGGCTAGTCCGTTATCAACTTGAAAA AGTGGGACCGAGTCGGTCC-3′ (SEQ ID NO: 3)), or may contain one or more nucleotide substitutions. In some embodiments, the heterologous object sequence has at least 90% identity, e.g., at least 90%, 95%, 98%, 99%, or 100% identity, or comprises no more than 1, 2, 3, 4, or 5 positions of non-identity to the target site 3′ of the first strand nick (e.g., immediately 3′ of the first strand nick or up to 1, 2, 3, 4, or 5 nucleotides 3′ of the first strand nick), with the exception of any insertion, substitution, or deletion that may be written into the target site by the polypeptide. In some embodiments, the 3′ target homology domain contains at least 90% identity, e.g., at least 90%, 95%, 98%, 99%, or 100% identity, or comprises no more than 1, 2, 3, 4, or 5 positions of non-identity to the target site 5′ of the first strand nick (e.g., immediately 5′ of the first strand nick or up to 1, 2, 3, 4, or 5 nucleotides 3′ of the first strand nick).

Improved Template Nucleic Acids

Without wishing to be bound by theory, improving features of the interaction of the template nucleic acid (e.g., template RNA) with a polypeptide (e.g., a polypeptide comprising a reverse transcriptase (RT) or RT domain, e.g., a genome editing polypeptide as described herein) can improve methods of modifying DNA and/or systems comprising said components. In particular, improving target-primed reverse transcription of the template nucleic acid (e.g., template RNA) by a polypeptide (e.g., a polypeptide comprising a reverse transcriptase (RT) or RT domain, e.g., a genome editing polypeptide as described herein) can improve methods of modifying DNA and/or systems comprising said components.

Alterations

A template nucleic acid, e.g., template RNA, of the present disclosure may comprise an alteration relative to a corresponding original sequence. The alteration may change, e.g., improve, a characteristic of the interaction of the template nucleic acid (e.g., template RNA) with a polypeptide, e.g., a polypeptide comprising a reverse transcriptase (RT) or RT domain, e.g., a genome editing polypeptide as described herein. In some embodiments, the alteration improves a characteristic of target-primed reverse transcription (TPRT) of the template nucleic acid (e.g., template RNA) or a portion thereof, e.g., the heterologous object sequence. In some embodiments, the heterologous object sequence comprises an alteration relative to a corresponding original sequence.

In some embodiments, the alteration improves, e.g., increases, the speed of TPRT by a polypeptide, e.g., a polypeptide comprising a reverse transcriptase (RT) or RT domain, e.g., a genome editing polypeptide as described herein. In some embodiments, improving speed comprises increasing the processivity of the polypeptide (e.g., the RT or RT domain), the polymerization rate of the polypeptide (e.g., the RT or RT domain), or both.

In some embodiments, the alteration improves, e.g., increases, the fidelity of TPRT by a polypeptide, e.g., a polypeptide comprising a reverse transcriptase (RT) or RT domain, e.g., a genome editing polypeptide as described herein. In some embodiments, improving fidelity comprises decreasing the error rate of the polypeptide (e.g., the RT or RT domain).

In some embodiments, improving fidelity comprises preventing or decreasing the amount of incorporation of non-heterologous object sequence template RNA sequence into the target site in the DNA (e.g., the target site in a target genome). Without wishing to be bound by theory, it is thought that a polypeptide comprising an RT or RT domain might continue TPRT past the heterologous object sequence, e.g., to reverse transcribe sequences 5′ of the heterologous object sequence on the template RNA, e.g., a sequence that binds a target site (e.g., a second strand of a site in a target genome) or a sequence that binds a polypeptide (e.g., a genome editing polypeptide as described herein) comprising a reverse transcriptase (RT) or RT domain. In some embodiments, incorporation of sequences of the template RNA besides the heterologous object sequence may be undesirable. An advantage of systems as described herein is modification of target genomic DNA accompanied by little or no extraneous exogenous DNA incorporation. An alteration that prevents or decreases the amount of said incorporation is thus desirable.

In some embodiments, the alteration improves, e.g., increases, the speed and the fidelity of TPRT by a polypeptide, e.g., a polypeptide comprising a reverse transcriptase (RT) or RT domain, e.g., a genome editing polypeptide as described herein.

In some embodiments, the sequence of the template nucleic acid, e.g., template RNA, is designed to improve the activity of the writing domain, e.g., reverse transcription, e.g., to increase processivity or reduce error rate of the writing domain. In some embodiments, the primary sequence of the template nucleic acid, e.g., template RNA, may be modified to increase or decrease the frequency of particular nucleotides, e.g., A, T, C, U, or G. In some embodiments, the sequence of the template nucleic acid, e.g. template RNA, may be modified to increase or decrease the GC content of the template nucleic acid, e.g. template RNA. In some embodiments, the sequence of the template RNA may be modified to decrease the occurrence of uracil. In some embodiments, the primary sequence of the template nucleic acid, e.g., template RNA, may be optimized to increase or decrease the predicted formation of secondary or tertiary structures. In some embodiments, the optimization of the primary sequence of the template nucleic acid, e.g., template RNA, does not alter the amino acid sequence of any encoded proteins.

The alteration may be made relative to a corresponding original sequence. A template nucleic acid, e.g., template RNA, may comprise sequences from multiple different sources. A corresponding original sequence may be the source sequence for a portion or all of the template nucleic acid (e.g., template RNA). In some embodiments, the corresponding original sequence is a wild-type sequence, a disease-associated sequence, a mutant sequence, or a recombinant sequence (e.g., an engineered therapeutic polypeptide). In some embodiments, the alteration may be made relative to a naturally occurring sequence. For example, the alteration could be made relative to a functional wild-type copy of a gene. In some embodiments, the alteration may be made relative to a naturally occurring mutant sequence, e.g., a genetically defective copy of a gene sequence (e.g., a sequence with a disease associated genetic defect). In some embodiments, the alteration may be made relative to a synthetic or recombinant sequence that is not naturally found. For example, the alteration may be made relative to a man-made sequence, e.g., a gRNA or recombinant gene, known in the art. In some embodiments, the corresponding original sequence is a wild-type gene sequence, a wild-type mRNA sequence or reverse complement thereof, an original nucleic acid sequence encoding a mutant protein, original nucleic acid sequence encoding an artificial protein (e.g., fusion protein), or a sequence encoding a protective mutation, or a portion of any thereof.

In some embodiments, the alteration comprises an insertion or substitution with a canonical nucleotide. In other embodiments, the alteration comprises an insertion or substitution with a chemically modified nucleotide.

In some embodiments, the heterologous object sequence encodes a polypeptide or portion thereof or comprises a sequence that is the reverse complement of a sequence encoding a polypeptide or portion thereof. In some embodiments, the polypeptide or portion thereof is a wild-type protein or an artificial protein, e.g., a fusion protein. In some embodiments, the alteration comprises a change to the sequence encoding the polypeptide or portion thereof or to the sequence that is the reverse complement of a sequence encoding a polypeptide or portion thereof. In some embodiments, the alteration does not change the amino acid sequence of the polypeptide encoded by the heterologous object sequence. In some embodiments, the alteration comprises a change to the wobble position (the third position) of a codon encoding the polypeptide or portion thereof. In some embodiments, the alteration changes the amino acid sequence of the polypeptide encoded by the heterologous object sequence. In some embodiments, the alteration does not change the function of the polypeptide encoded by the heterologous object sequence (e.g., the polypeptide produced from the alteration containing heterologous object sequence is functional, or retains at least 50, 60, 70, 80, 90, or 100% of the function of the unaltered polypeptide). In some embodiments, the alteration is in a regulatory sequence, e.g., a promoter sequence or enhancer sequence.

In some embodiments, RNA templates with sense (coding) and antisense heterologous object sequences can be altered. In some embodiments, the template comprises a coding strand of a gene, such that the RNA of the heterologous object sequence is the same sequence as would be transcribed from the genome upon integration to produce a product(s) from the heterologous object sequence (e.g., mRNA encoding a therapeutic protein). When in the same sense as the desired mRNA, the primary sequence may be codon optimized, e.g., wherein a plurality of (e.g., all possible) primary sequences encoding the same amino acid sequence are analyzed to optimize for a particular function, e.g., using the most commonly occurring codons to increase (e.g., maximize) expression. In some embodiments, the optimization is performed around secondary structure, where a plurality of (e.g., all possible) primary sequences encoding the same amino acid sequence are analyzed and a primary sequence resulting in less secondary structure, e.g., a sequence with a higher free folding energy, is selected. In some embodiments, the optimization is performed around secondary structure, where a plurality of (e.g., all possible) primary sequences encoding the same amino acid sequence are analyzed and a primary sequence resulting in more secondary structure, e.g., a sequence with a lower free folding energy, is selected.

In some embodiments, the template comprises the antisense strand of a gene, such that the template RNA comprises the reverse complementary sequence of what would be transcribed from the genome upon integration to produce a product(s) from the heterologous object sequence (e.g., mRNA encoding a therapeutic protein). When in the antisense of the desired mRNA, the primary sequence may be optimized by considering the codons of the reverse complementary strand, where a plurality of (e.g., all possible) primary sequences corresponding to a sense strand that would encode the same amino acid sequence are analyzed to optimize for a particular function, e.g., using the most commonly occurring codons to increase (e.g., maximize) expression. In some embodiments, the optimization is performed around secondary structure, where a plurality of (e.g., all possible) primary sequences corresponding to a sense strand that would encode the same amino acid sequence are analyzed and a primary sequence resulting in less secondary structure, e.g., a sequence with a higher free folding energy, is selected. In some embodiments, the optimization is performed around secondary structure, where a plurality of (e.g., all possible) primary sequences corresponding to a sense strand that would encode the same amino acid sequence are analyzed and a primary sequence resulting in more secondary structure, e.g., a sequence with a lower free folding energy, is selected.

In some embodiments, the template nucleic acid (e.g., template RNA) further comprises one or more additional alterations (e.g., a second, third, fourth, or fifth alteration) that improve the speed, fidelity, or both of TPRT by a polypeptide (e.g., a polypeptide comprising a reverse transcriptase (RT) or RT domain, e.g., a genome editing polypeptide as described herein). In some embodiments, the template nucleic acid (e.g., template RNA) further comprises one or more additional alterations (e.g., a second, third, fourth, or fifth alteration) that do not affect the speed, fidelity, or both of TPRT.

In some embodiments, an alteration comprises one or more substitutions, insertions, or deletions in the nucleic acid sequence of the heterologous object sequence relative to the original sequence. In some embodiments, an alteration comprises substitution of at least 1, 2, 3, 4, 5, 6, 7, 8, 9, 10, 15, 20, 30, 40, 50, 60, 70, 80, 90, or 100 nucleotides (and optionally no more than 3, 4, 5, 6, 7, 8, 9, 10, 15, 20, 30, 40, 50, 60, 70, 80, 90, 100, or 150). In some embodiments, an alteration comprises insertion of at least 1, 2, 3, 4, 5, 6, 7, 8, 9, 10, 15, 20, 30, 40, 50, 60, 70, 80, 90, or 100 nucleotides (and optionally no more than 3, 4, 5, 6, 7, 8, 9, 10, 15, 20, 30, 40, 50, 60, 70, 80, 90, 100, or 150). In some embodiments, an alteration comprises deletion of at least 1, 2, 3, 4, 5, 6, 7, 8, 9, 10, 15, 20, 30, 40, 50, 60, 70, 80, 90, or 100 nucleotides (and optionally no more than 3, 4, 5, 6, 7, 8, 9, 10, 15, 20, 30, 40, 50, 60, 70, 80, 90, 100, or 150).

In some embodiments, the corresponding original sequence comprises a self-complementary sequence, e.g., wherein the self-complementary sequence is capable of hybridizing to itself, e.g., to form secondary structures. For instance, the self-complementary sequence may comprise a first self-complementary region and a second self-complementary region that can bind each other, e.g., to form a hairpin. In some embodiments, the first self- and second self-complementary regions are perfectly complementary, and in other embodiments they have one or more mismatch or bulge. In some embodiments, the corresponding original sequence comprises a first self-complementary region and a second self-complementary region which are capable of hybridizing to one another, e.g., to form a hairpin structure. In some embodiments, the alteration is in the first self-complementarity region and reduces complementarity between the first self-complementary region and the second self-complementary region. In some embodiments, reducing complementarity between the first and second self-complementary regions disrupts, e.g., decreases or prevents, the formation of secondary structure, e.g., hairpin structures. In some embodiments, the first self-complementary region is at least 4, 5, 6, 7, 8, 9, 10, 11, 12, 13, 14, 15, 16, 17, 18, 19, or 20 nucleotides long (and optionally no more than 5, 6, 7, 8, 9, 10, 11, 12, 13, 14, 15, 16, 17, 18, 19, 20, or 30 nucleotides long). In some embodiments, the second self-complementary region is at least 4, 5, 6, 7, 8, 9, 10, 11, 12, 13, 14, 15, 16, 17, 18, 19, or 20 nucleotides long (and optionally no more than 5, 6, 7, 8, 9, 10, 11, 12, 13, 14, 15, 16, 17, 18, 19, 20, or 30 nucleotides long). In some embodiments, the first and second self-complementary regions are capable of hybridizing to form a double-stranded or partially double-stranded structure (e.g., a hairpin) comprising at least 4, 5, 6, 7, 8, 9, 10, 11, 12, 13, 14, 15, 16, 17, 18, 19, or 20 nucleotides long (and optionally no more than 5, 6, 7, 8, 9, 10, 11, 12, 13, 14, 15, 16, 17, 18, 19, 20, or 30 nucleotides long) of each self-complementary region. In some embodiments, the first self-complementary region and the second self-complementary region of the original sequence have no more than 1, 2, 3, 4, 5, 6, 7, or 8 positions of non-complementarity (e.g., mismatches or bulges). In some embodiments, the first self-complementary region and the second self-complementary region of the original sequence comprise a region of perfect complementarity of at least 3, 4, 5, 6, 7, 8, 9, 10, 11, 12, 13, 14, 15, 16, 17, 18, 19, or 20 base pairs in length. In some embodiments, the first self-complementary region and the second self-complementary region of the original sequence comprise a region of partial complementarity, wherein the positions of non-complementarity comprise one or more (e.g., all) wobble base pairs (e.g., G to U, hypoxanthine (I) to U, I to A, or I to C). In some embodiments, the alteration disrupts a G to C base pairing of the first self-complementary region and the second self-complementary region. In some embodiments, the alteration disrupts an A to U base pairing of the first self-complementary region and the second self-complementary region. In some embodiments, the alteration disrupts an G to U base pairing of the first self-complementary region and the second self-complementary region. In some embodiments, the first and second self-complementary regions are separated by about 3, 4, 5, 6, 7, 8, 9, or 10 or more nucleotides. In some embodiments, a hairpin formed by the first and second self-complementary regions comprises a loop of about 3, 4, 5, 6, 7, 8, 9, or 10 or more nucleotides.

In some embodiments, the alteration reduces the predicted secondary structure of the template nucleic acid, e.g., template RNA, e.g., the heterologous object sequence. Such decreases may be assessed, for example, as described below. In some embodiments, the alteration eliminates a hairpin structure from the heterologous object sequence. In some embodiments, the alteration decreases the length of a double stranded region of a hairpin structure in the heterologous object sequence, e.g., by 1, 2, 3, 4, 5, 6, 7, 8, 9, or 10 base pairs. For example, in some embodiments, the alteration reduces the number of predicted base pairs to below 10, 9, 8, 7, 6, 5, 4, 3, or 2.

Secondary structures in a template RNA can also be predicted in silico by software tools, e.g., the RNAstructure tool available on the world wide web at rna.urmc.rochester.edu/RNAstructureWeb (Bellaousov et al. Nucleic Acids Res 41:W471-W474 (2013); incorporated by reference herein in its entirety), e.g., to determine secondary structures for selecting modifications, e.g., hairpins, stems, and/or bulges.

In some embodiments, the template RNA is sequence optimized, e.g., to reduce secondary structures, for example, as measured by RNAstructure per the methods of Turner and Mathews (2009) Nucleic Acids Res 38:D280-282. In some embodiments, the template RNA is sequence optimized, e.g., to reduce secondary structure, for example, as measured in vitro by SHAPE-MaP, e.g., as described in Siegfried et al. Nat Methods (2014) 11:959-965 (incorporation by reference herein in its entirety). In some embodiments, the template RNA is sequence optimized, e.g., to reduce secondary structure, for example, as measured in cells by DMS-MaPseq, e.g., as described in Zubradt et al. Nat Methods (2017) 14:75-82 (incorporated by reference herein in its entirety).

In some embodiments, the alteration replaces a G with an A, a G with a T, or a G with a U. In some embodiments, the alteration replaces a C with an A, a C with a T, or a C with a U. In some embodiments, the alteration changes the GC content of the template nucleic acid, e.g., template RNA, e.g., the heterologous object sequence. In some embodiments, the alteration decreases the GC content level of the heterologous object sequence (e.g., by at least 1, 2, 3, 4, 5, 6, 7, 8, 9, 10, 15, 20, 25, or 30%). In some embodiments, the altered heterologous object sequence has a GC content of less than 70%, 60%, 50%, 40%, 30%, or 20%. In some embodiments, the altered template nucleic acid has a GC content of less than 70%, 60%, 50%, 40%, 30%, or 20%.

In some embodiments, the alteration eliminates or shortens a repetitive sequence in the heterologous object sequence, e.g., a single, di- or tri-nucleotide repeat. In some embodiments, the repetitive sequence is shortened by at least 1, 2, 3, 4, 5, 6, 7, 8, 9, 10, 15, 20, 25, 30, 35, 40, 45, or 50% its total length, or by at least 1, 2, 3, 4, 5, 6, 7, 8, 9, 10, 15, 20, 25, 30, 35, 40, 45, or 50 nucleotides. In some embodiments, the heterologous object sequence does not comprise any single nucleotide repeat that is longer than 2, 3, 4, 5, 6, 7, 8, 9, or 10, nucleotides. In some embodiments, the heterologous object sequence does not comprise any di- or tri-nucleotide repeat that is longer than 2, 3, 4, 5, 6, 7, 8, 9, or 10 repeats.

In some embodiments, optimizing the energy level of the structures a template nucleic acid, e.g., template RNA, can improve methods of modifying DNA and/or systems comprising said components, e.g., improve TPRT of the template RNA (e.g., the heterologous object sequence). In some embodiments, the alteration changes the minimal energy structure of the template RNA to between −280 and −480 kcal/mol (e.g., between −280 and −300, −300 and −320, −320 and −340, −340 and −360, 360 and −380, −380 and −400, −400 and −420, −420 and −440, −440 and −460, −460 and −480 kcal/mol). In embodiments, the energy structures of the template RNA are measured by RNAstructure, e.g., as described in Turner and Mathews (2009) Nucleic Acids Res 38:D280-282 (incorporated by reference herein in its entirety). In some embodiments, the alteration changes (e.g., increases) the minimum free energy of folding (e.g., predicted minimum free energy of folding) of the template RNA, e.g., as measured by a tool such as RNAStructure as described in Example 2. In some embodiments, the alteration increases the minimum free energy of folding (e.g., predicted minimum free energy of folding) of the template RNA by at least 1, 2, 3, 4, 5, 6, 7, 8, 9, 10, 15, 20, 25, 30, 35, 40, 45, 50, 55, 60, 65, 70, 75, 80, 85, 90, 95, or 100 kcal/mol, e.g., as measured by a tool such as RNAStructure as described in Example 2. In some embodiments, the alteration increases the minimum free energy of folding (e.g., predicted minimum free energy of folding) of the template RNA by at least 5, 10, 15, 20, 25, 30, 35, 40, 45, 50, 55, 60, 65, 70, 75, 80, 85, 90, 95, or 100% relative to the minimum free energy of folding of an otherwise similar template RNA lacking the alteration, e.g., as measured by a tool such as RNAStructure as described in Example 2.

In some embodiments, contacting a plurality of cells with a system or a template RNA comprising an alteration produces a higher number of genomic modifications comprising the heterologous object sequence compared to contacting a similar plurality of cells with an otherwise similar system comprising a template RNA not comprising the alteration. In some embodiments, said contacting produces at least 1, 2, 3, 4, 5, 6, 7, 8, 9, 10, 20, 30, 40, 50, 60, 70, 80, 90, 100, 120, 140, 160, 180, 200, 300, 400, 500, or 1000% more genomic modifications comprising the heterologous object sequence compared to contacting a similar plurality of cells with an otherwise similar system comprising a template RNA not comprising the alteration.

In some embodiments, contacting a plurality of cells with a system or template RNA comprising an alteration produces a higher fraction of complete integrations into genomic DNA (e.g., comprising the entire heterologous object sequence) compared to contacting a similar plurality of cells with an otherwise similar system comprising a template RNA not comprising the alteration. For example, a complete integration could comprise integrating an entire gene of the heterologous object sequence being integrated into the genomic DNA. As a further example, a complete integration could comprise inserting the desired number of nucleotide repeats into the genomic DNA. In some embodiments, contacting a plurality of cells with a system or template RNA comprising an alteration produces a lower fraction of incomplete integrations into genomic DNA (e.g., comprising only a portion of the heterologous object sequence) compared to contacting a similar plurality of cells with an otherwise similar system comprising a template RNA not comprising the alteration. For example, an incomplete integration could comprise integrating only part of the coding sequence of a gene of the heterologous object sequence into the genomic DNA, or a non-preferred (e.g., lower) number of nucleotide repeats into the genomic DNA. In some embodiments, contacting a plurality of cells with a system or template RNA comprising an alteration produces an at least 1, 2, 3, 4, 5, 6, 7, 8, 9, 10, 20, 30, 40, 50, 60, 70, 80, 90, 100, 120, 140, 160, 180, 200, 300, 400, 500, or 1000% higher fraction of complete integrations into genomic DNA. In some embodiments, contacting a plurality of cells with a system or template RNA comprising an alteration produces an at least 1, 2, 3, 4, 5, 6, 7, 8, 9, 10, 20, 30, 40, 50, 60, 70, 80, 90, or 100% lower fraction of incomplete integrations into genomic DNA.

In some embodiments, the reverse transcriptase domain is able to complete at least about 30% or 50% of integrations in cells. The percent of complete integrations can be measured by dividing the number of substantially full-length integration events (e.g., genomic sites that comprise at least 98% of the expected integrated sequence) by the number of total (including substantially full-length and partial) integration events in a population of cells. In embodiments, the integrations in cells is determined (e.g., across the integration site) using long-read amplicon sequencing, e.g., as described in Karst et al. (2020) bioRxiv doi.org/10.1101/645903 (incorporated by reference herein in its entirety).

In embodiments, quantifying integrations in cells comprises counting the fraction of integrations that contain at least about 75%, 80%, 85%, 90%, 95%, 96%, 97%, 98%, 99%, or 100% of the DNA sequence corresponding to the template RNA (e.g., a template RNA having a length of at least 0.05, 0.1, 0.5, 0.6, 0.7, 0.8, 0.9, 1, 1.5, 2, 3, 4, or 5 kb, e.g., a length between 0.5-0.6, 0.6-0.7, 0.7-0.8, 0.8-0.9, 1.0-1.2, 1.2-1.4, 1.4-1.6, 1.6-1.8, 1.8-2.0, 2-3, 3-4, or 4-5 kb). In some embodiments, contacting a plurality of cells with a system or template RNA comprising an alteration produces a higher fraction of total genetic alterations at the target site compared to contacting a similar plurality of cells with an otherwise similar system comprising a template RNA not comprising the alteration (e.g., higher by 5%, 10%, 20%, 30%, 40%, 50%, 60%, 70%, 80%, 90%, or 100%).

In some embodiments, a template nucleic acid, e.g., template RNA, comprises some or all of a gRNA. gRNA molecules may be modified by the addition or subtraction of the naturally occurring structural components, e.g., hairpins. In some embodiments, a template nucleic acid, e.g., template RNA, may comprise an alteration deleting one or more 3′ hairpin elements from a gRNA, e.g., as described in WO2018106727, incorporated herein by reference in its entirety. In some embodiments, an alteration adds an additional hairpin structure to a gRNA, e.g., an added hairpin structure in the spacer region, e.g., that increases specificity of a CRISPR-Cas system, e.g., as taught by Kocak et al. Nat Biotechnol 37(6):657-666 (2019). Additional modifications, including examples of shortened gRNA and specific modifications improving in vivo activity, can be found in US20190316121, incorporated herein by reference in its entirety.

In some embodiments, a template nucleic acid (e.g., template RNA), e.g., as described herein, comprises one or more blocking moieties at one or both ends. In some embodiments, a template RNA comprising the blocking moiety has a half-life that is at least 10%, 20%, 30%, 40%, 50%, 60%, 70%, 80%, 90%, or 100% longer than that of an otherwise similar template RNA lacking the blocking moiety, e.g., under cell culture conditions described herein.

In some embodiments, the template nucleic acid (e.g., template RNA) comprises a 3′ end blocking moiety, e.g., a mutation, modification, or moiety that blocks an exoribonuclease from degrading at least a portion of the template nucleic acid (e.g., a portion comprising a 3′ target homology domain, e.g., as described herein). In some embodiments, the 3′ end blocking moiety results in the formation of an exoribonuclease resistant RNA secondary and/or tertiary structure (e.g., a hairpin, xrRNA, triplex, pseudoknot, or G-quadruplex). In some embodiments, the 3′ end blocking moiety comprises a chemical modification (e.g., TEG, biotin, spermine, or a ligand to be delivered (e.g., GalNAc)).

In some embodiments, the template nucleic acid (e.g., template RNA) comprises a 5′ end blocking moiety, e.g., a mutation, modification, or moiety that blocks an exoribonuclease from degrading at least a portion of the template nucleic acid (e.g., a portion comprising a sequence that binds to a target site, e.g., as described herein). In some embodiments, the 5′ end blocking moiety results in the formation of an exoribonuclease resistant RNA secondary and/or tertiary structure (e.g., a hairpin, xrRNA, triplex, pseudoknot, or G-quadruplex). In some embodiments, the 5′ end blocking moiety comprises a chemical modification (e.g., TEG, biotin, spermine, or a ligand to be delivered (e.g., GalNAc)).

Dual Templates

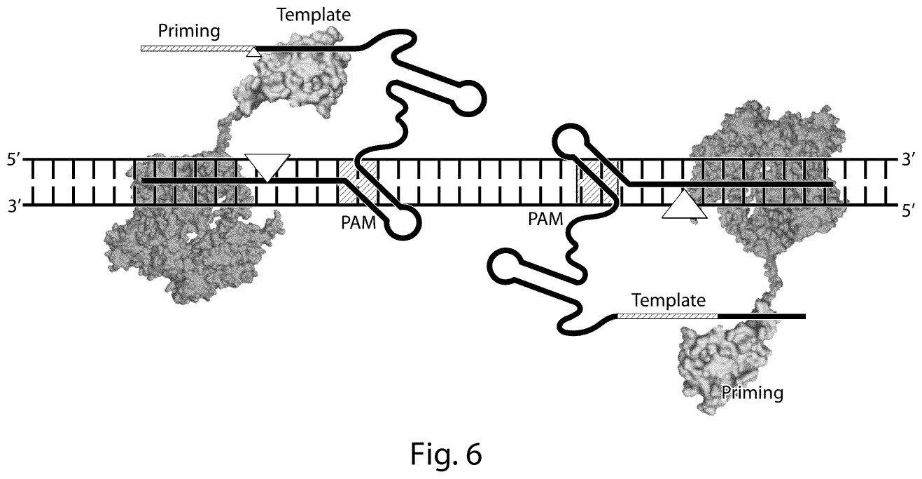

In some embodiments, an editing system as described herein comprises a plurality (e.g., two or more) of template nucleic acids (e.g., template RNAs). In some embodiments, an editing system comprises: (i) a first template nucleic acid (e.g., a first template RNA) that comprises a sequence that binds to a first target site in a first strand of a DNA; and (ii) a second template nucleic acid (e.g., a second template RNA) that comprises a sequence that binds to a second target site in a second strand of the DNA (see, e.g., FIG. 6). In some embodiments, the first target site and the second target site are antiparallel relative to each other. In some embodiments, the first and second target sites are located about 5, 6, 7, 8, 9, 10, 11, 12, 13, 14, 15, 16, 17, 18, 19, 20, 25, 30, 40, 50, 60, 70, 80, 90, 100, 150, 200, 300, 400, 500, 600, 700, 800, 900, 1000, 1500, 2000, 2500, 3000, 4000, 5000, 6000, 7000, 8000, 9000, or 10,000 nucleotides apart from each other on the DNA. In some embodiments, the first and second target sites are located about 5-10, 10-20, 20-30, 30-40, 40-50, 50-60, 60-70, 70-80, 80-90, 90-100, 100-200, 200-300, 300-400, 400-500, 500-600, 600-700, 700-800, 800-900, 900-1000, 1000-2000, 2000-3000, 3000-4000, 4000-5000, 5000-6000, 6000-7000, 7000-8000, 8000-9000, or 9000-10,000 nucleotides apart from each other on the DNA. In some embodiments, the first target site and the second target site share at least 75%, 80%, 85%, 90%, 95%, 96%, 97%, 98%, 99%, or 100% sequence identity.

In some embodiments, the first template nucleic acid further comprises: a sequence that binds to a polypeptide (e.g., as described herein, e.g., comprising a reverse transcriptase (RT) domain and, optionally, an endonuclease domain, e.g., a nickase domain), a heterologous object sequence, and optionally a 3′ target homology domain. In some embodiments, the second template nucleic acid further comprises: a sequence that binds to a polypeptide (e.g., as described herein, e.g., comprising an RT domain), a heterologous object sequence, and optionally a 3′ target homology domain. In certain embodiments, the sequences that bind to the polypeptide of the first and second template nucleic acids share at least 75%, 80%, 85%, 90%, 95%, 96%, 97%, 98%, 99%, or 100% sequence identity. In certain embodiments, the heterologous object sequences of the first and second template nucleic acids share at least 75%, 80%, 85%, 90%, 95%, 96%, 97%, 98%, 99%, or 100% sequence identity. In certain embodiments, the 3′ target homology domains of the first and second template nucleic acids share at least 75%, 80%, 85%, 90%, 95%, 96%, 97%, 98%, 99%, or 100% sequence identity.

In some embodiments, the heterologous object sequences of the first and second template nucleic acids are complementary in the regions corresponding to the terminal nucleotides that will be reverse transcribed by the polypeptide(s) of the system (e.g., polypeptides comprising reverse transcriptase domains, e.g., as described herein), e.g., such that the 3′ ends of the ssDNA regions polymerized during TPRT onto the first and second strands of the target nucleic acid also comprise terminal complementarity. In some embodiments, terminal complementarity of the ssDNA regions polymerized during TPRT onto the first and second strands of the target nucleic acid results in intermolecular annealing of the newly synthesized regions. In some embodiments, annealing of a newly synthesized first and second strand results in priming of the remaining ssDNA regions, e.g., a first ssDNA anneals to a second ssDNA. In some embodiments, a DNA-dependent DNA polymerase activity of the genome editing system (e.g., provided by an RT domain of the system that is also capable of DNA-dependent DNA polymerization) enables continued synthesis of the first strand, e.g., by using the 3′ end of the first ssDNA as a DNA primer and the second ssDNA as a template molecule for DNA polymerization. In some embodiments, the system enables continued synthesis of the second strand, e.g., by using the 3′ end of the second ssDNA as a DNA primer and the first ssDNA as a template molecule for DNA polymerization. In some embodiments, complete synthesis of both strands enables resolution of the target edit to be independent of any DNA polymerase endogenous to the host cell.

In some embodiments, the first template nucleic acid and the second template nucleic acid each bind to a copy of the same polypeptide (e.g., a polypeptide comprising a reverse transcriptase (RT) domain and optionally an endonuclease domain, e.g., nickase domain). In some embodiments, the first target site is located adjacent to or within 1, 2, 3, 4, 5, 6, 7, 8, 9, or nucleotides of a first protospacer adjacent motif (PAM). In some embodiments, the second target site is located adjacent to or within 1, 2, 3, 4, 5, 6, 7, 8, 9, or 10 nucleotides of a second PAM. In some embodiments, the first PAM and the second PAM share at least 75%, 80%, 85%, 90%, 95%, 96%, 97%, 98%, 99%, or 100% sequence identity. In some embodiments, the first template nucleic acid is configured to drive reverse transcription by a polypeptide towards the sequence antiparallel to the second target site. In some embodiments, the second template nucleic acid is configured to drive reverse transcription by a polypeptide towards the sequence antiparallel to the first target site.

In some embodiments, the first template nucleic acid comprises a sequence at its 5′ end capable of hybridizing to (e.g., complementary to) a sequence at the 5′ end of the second template nucleic acids. In some embodiments, TPRT involving the first and second template nucleic acids produces cDNAs comprising regions capable of hybridization (e.g., complementary to each other).

RT Terminators

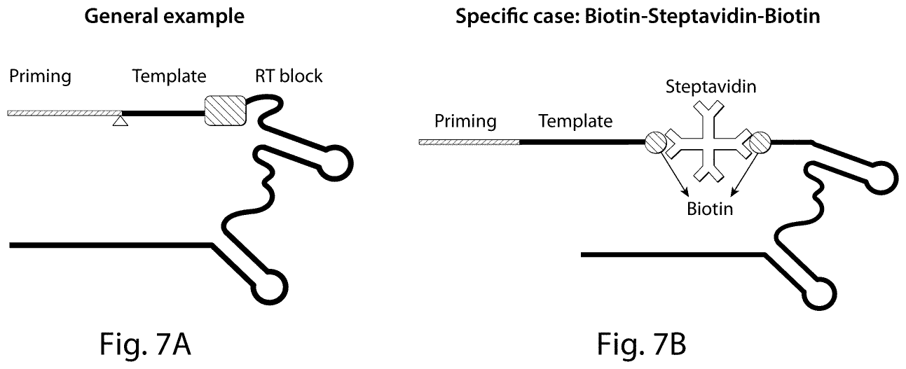

In some embodiments, the template nucleic acid, e.g., template RNA, may comprise one or more moieties (referred to herein as reverse transcriptase (RT) termination moieties that terminate the action of the writing domain of a polypeptide (e.g., a polypeptide comprising an RT domain, e.g., a genome editing polypeptide as described herein), e.g., the reverse transcriptase domain, e.g., as described herein (see, e.g., FIG. 7A). In some embodiments, an RT termination moiety comprises one or more nucleic acids (e.g., a reverse transcriptase termination site as described below). In some embodiments, an RT termination moiety comprises a non-nucleic acid molecule.

In some embodiments, an RT termination moiety comprises a protein, e.g., an avidin moiety, e.g., streptavidin moiety, e.g., bound to a corresponding ligand (e.g., a biotin moiety) attached to the template nucleic acid (see, e.g., FIG. 7B). In certain embodiments, a template nucleic acid comprises a first nucleic acid (e.g., RNA) segment (e.g., comprising a sequence that binds to a target site in a DNA and/or a sequence that binds the polypeptide, e.g., as described herein) bound to a first biotin moiety bound to the streptavidin moiety, e.g., at the 3′ end of the sequence that binds to the polypeptide. In certain embodiments, a template nucleic acid comprises a second nucleic acid (e.g., RNA) segment (e.g., comprising a heterologous object sequence and/or a 3′ target homology domain, e.g., as described herein) bound to a second biotin moiety bound to the streptavidin moiety, e.g., at the 5′ end of the heterologous object sequence.

In some embodiments, the RT termination moiety comprises an artificially stabilized hairpin sequence. In some embodiments, the RT termination moiety comprises a modification internal to the template nucleic acid. In certain embodiments, the RT termination moiety comprises a spacer (e.g., a C3 spacer or tri/hexa-ethylene glycol spacer). In certain embodiments, the RT termination moiety comprises a trizole moiety (e.g., produced by click chemistry).

In some embodiments, the RT termination moiety comprises a structure as shown in Table 8. In some embodiments, the RT termination moiety comprises a structure as shown in Table 9.

In some embodiments, the template nucleic acid, e.g., template RNA, may comprise one or more sequences which terminate the action of the writing domain of a polypeptide (e.g., a polypeptide comprising an RT domain, e.g., as described herein, e.g., a genome editing polypeptide as described herein), e.g., the reverse transcriptase domain. In some embodiments, said one or more sequences comprise one or more reverse transcriptase termination sites (also referred to herein as RT terminators or RT terminator sequences). In some embodiments, said one or more sequences comprise one or more stem-loops or structural features preventing writing domain read-through, e.g., reverse transcriptase domain read-through. In some embodiments, the one or more RT terminators may be located 5′ of the heterologous object sequence. In some embodiments, the RT terminator is situated such that the heterologous object sequence will be reverse transcribed by the RT followed by termination of polymerization. In some embodiments, the one or more RT terminators may be in the 5′ end of the heterologous object sequence. Reverse transcriptase termination sites can also be found empirically by following the methods of Kielpinski et al. Methods Mol Biol 1038:213-231 (2013) to detect where DNA molecules reverse transcribed from RNA molecules, e.g., cDNA molecules, terminate relative to the complete RNA molecule. Sequence analysis of the context of the RNA molecule in the region where the cDNA ends can be used to located an RT termination element, e.g., a secondary structure that leads to termination of polymerization by RT. In some embodiments, the inclusion of one or more RT terminators prevents the reverse transcription (e.g., and subsequent integration) of portions of the template RNA, e.g., portions downstream of the desired RT template (e.g., heterologous object sequence), e.g., a sequence that binds to a polypeptide as described herein (e.g., a polypeptide comprising an RT domain, e.g., a genome editing polypeptide as described herein), e.g., a gRNA scaffold.

In some embodiments, the RT terminator sequence is situated between the heterologous object sequence and an adjacent component of the template nucleic acid (e.g., template RNA), e.g., a sequence that binds a target site (e.g., a second strand of a site in a target genome) or a sequence that binds the polypeptide (e.g., a polypeptide comprising a reverse transcriptase (RT) or RT domain, e.g., a genome editing polypeptide as described herein). In some embodiments, the RT terminator sequence is situated 3′ of (i) the sequence that binds a target site, (ii) the sequence that binds the polypeptide, or both (i) and (ii). In some embodiments, the RT terminator sequence is situated no more than 1, 2, 3, 4, 5, 6, 7, 8, 9, 10, 11, 12, 13, 14, 15, 16, 17, 18, 19, 20, 30, 40, or 50 nucleotides from the sequence that binds a target site. In some embodiments, the RT terminator sequence is situated directly adjacent to the sequence that binds a target site. In some embodiments, the RT terminator sequence is situated no more than 1, 2, 3, 4, 5, 6, 7, 8, 9, 10, 11, 12, 13, 14, 15, 16, 17, 18, 19, 20, 30, 40, or 50 nucleotides from the sequence that binds the polypeptide, e.g., the genome editing polypeptide as described herein. In some embodiments, the RT terminator sequence is situated directly adjacent to the sequence that binds the polypeptide. In some embodiments, the RT terminator sequence is situated no more than 1, 2, 3, 4, 5, 6, 7, 8, 9, 10, 11, 12, 13, 14, 15, 16, 17, 18, 19, 20, 30, 40, or 50 nucleotides from the heterologous object sequence. In some embodiments, the RT terminator sequence is situated directly adjacent to the heterologous object sequence. In some embodiments, the RT terminator sequence is 5′ of the heterologous object sequence.

In some embodiments, the RT terminator sequence is situated within the heterologous object sequence, e.g., at the 5′ end of the heterologous object sequence, e.g., no more than 1, 2, 3, 4, 5, 6, 7, 8, 9, 10, 11, 12, 13, 14, 15, 16, 17, 18, 19, 20, 30, 40, or 50 nucleotides from the 5′ end of heterologous object sequence. In some embodiments, the RT terminator sequence is not situated in a protein coding sequence of the heterologous object sequence.

In some embodiments, including one or more RT terminators in a template RNA decreases or prevents incorporation of non-heterologous object sequence (e.g., gRNA scaffold, vector backbone, and/or ITRs) into TPRT product. In some embodiments, contacting a plurality of cells with a system comprising a template RNA comprising one or more RT terminators in produces fewer genomic modifications comprising template RNA sequence that is not the heterologous object sequence compared to contacting a similar plurality of cells with a similar system comprising a template RNA not comprising the RT terminator sequence. In some embodiments, contacting a plurality of cells with the system produces at least 1, 2, 3, 4, 5, 6, 7, 8, 9, 10, 20, 30, 40, 50, 60, 70, 80, 90, or 100% fewer genomic modifications comprising template RNA sequence that is not the heterologous object sequence compared to contacting a similar plurality of cells with a similar system comprising a template RNA not comprising the RT terminator sequence (e.g., as determined by long-read amplicon sequencing of the target site, e.g., as described in Karst et al. bioRxiv doi.org/10.1101/645903 (2020) (incorporated herein by reference in its entirety)).

In some embodiments, wherein contacting a plurality of cells with the system produces fewer genomic modifications comprising (i) the sequence that binds the target site or (ii) the sequence that binds the polypeptide, e.g., the genome editing polypeptide as described herein, compared to contacting a similar plurality of cells with a similar system comprising a template RNA not comprising the RT terminator sequence. In some embodiments, contacting a plurality of cells with the system produces at least 1, 2, 3, 4, 5, 6, 7, 8, 9, 10, 20, 30, 40, 50, 60, 70, 80, 90, or 100% fewer genomic modifications comprising (i) the sequence that binds the target site or (ii) the sequence that binds the polypeptide, e.g., the genome editing polypeptide as described herein, compared to contacting a similar plurality of cells with a similar system comprising a template RNA not comprising the RT terminator sequence (e.g., as determined by long-read amplicon sequencing of the target site, e.g., as described in Karst et al. bioRxiv doi.org/10.1101/645903 (2020) (incorporated herein by reference in its entirety)). In some embodiments, reverse transcription (RT) of the heterologous object sequence by a polypeptide, e.g., a genome editing polypeptide as described herein, produces fewer DNA products comprising template RNA sequence that is not the heterologous object sequence compared to RT of a similar heterologous object sequence not comprising the RT terminator sequence. In some embodiments reverse transcription (RT) of the heterologous object sequence by a polypeptide, e.g., a genome editing polypeptide as described herein, produces at least 1, 2, 3, 4, 5, 6, 7, 8, 9, 10, 20, 30, 40, 50, 60, 70, 80, 90, or 100% fewer DNA products comprising template RNA sequence that is not the heterologous object sequence compared to RT of a similar heterologous object sequence not comprising the RT terminator sequence (e.g., as determined by long-read amplicon sequencing of the target site, e.g., as described in Karst et al. bioRxiv doi.org/10.1101/645903 (2020) (incorporated herein by reference in its entirety)). In some embodiments reverse transcription (RT) of the heterologous object sequence by a polypeptide, e.g., a genome editing polypeptide as described herein, produces fewer DNA products comprising (i) the sequence that binds the target site or (ii) the sequence that binds the polypeptide, e.g., the genome editing polypeptide as described herein, compared to RT of a similar heterologous object sequence not comprising the RT terminator sequence. In some embodiments reverse transcription (RT) of the heterologous object sequence by a polypeptide, e.g., a genome editing polypeptide as described herein, produces at least 1, 2, 3, 4, 5, 6, 7, 8, 9, 10, 20, 30, 40, 50, 60, 70, 80, 90, or 100% fewer DNA products comprising (i) the sequence that binds the target site or (ii) the sequence that binds the polypeptide, e.g., the genome editing polypeptide as described herein, compared to RT of a similar heterologous object sequence not comprising the RT terminator sequence (e.g., as determined by long-read amplicon sequencing of the target site, e.g., as described in Karst et al. bioRxiv doi.org/10.1101/645903 (2020) (incorporated herein by reference in its entirety)).

In some embodiments, a template nucleic acid, e.g., template RNA, comprises a 5′ target homology domain. In some embodiments, reverse transcription (RT) of the heterologous object sequence by a polypeptide, e.g., a genome editing polypeptide as described herein, produces fewer DNA products comprising the 5′ target homology domain compared to RT of a similar heterologous object sequence not comprising the RT terminator sequence. In some embodiments, reverse transcription (RT) of the heterologous object sequence by a polypeptide, e.g., a genome editing polypeptide as described herein, produces at least 1, 2, 3, 4, 5, 6, 7, 8, 9, 10, 20, 30, 40, 50, 60, 70, 80, 90, or 100% fewer DNA products comprising the 5′ target homology domain compared to RT of a similar heterologous object sequence not comprising the RT terminator sequence (e.g., as determined by long-read amplicon sequencing of the target site, e.g., as described in Karst et al. bioRxiv doi.org/10.1101/645903 (2020) (incorporated herein by reference in its entirety)).

In some embodiments, an RT terminator sequence comprises a sequence that adopts a secondary structure under physiological conditions. Without wishing to be bound by theory, secondary structures in the template RNA may impede the progress of TPRT, e.g., by a polypeptide (e.g., a polypeptide comprising an RT domain, e.g., a genome editing polypeptide as described herein), thereby promoting termination of TPRT and decreasing or preventing reverse transcription of sequence beyond the secondary structure. In some embodiments, the RT terminator sequence comprises a first self-complementary region and a second self-complementary region, e.g., a palindromic or partially palindromic sequence. In some embodiments, self-complementary regions comprise completely or partially complementary nucleic acid sequences such that the self-complementary regions hybridize, e.g., under stringent conditions and/or physiological conditions. In some embodiments, the first self-complementary region and a second self-complementary region are partially complementary to one another, e.g., they are complementary to one another in at least 50, 60, 70, 80, 90, 95, or 99% of positions (but not completely complementary at every position) and/or they comprise at least 1, 2, 3, 4, 5, 6, 7, 8, 9, or 10 positions of non-complementarity. In some embodiments, the first self-complementary region and a second self-complementary region are completely complementary to one another. In some embodiments, the first self-complementary region and a second self-complementary region comprise a region of complete complementarity and a region of partial complementarity. In some embodiments, the RT terminator sequence adopts a secondary or tertiary structure comprising one or more hairpins, e.g., under stringent conditions.

Self-Annealing Elements

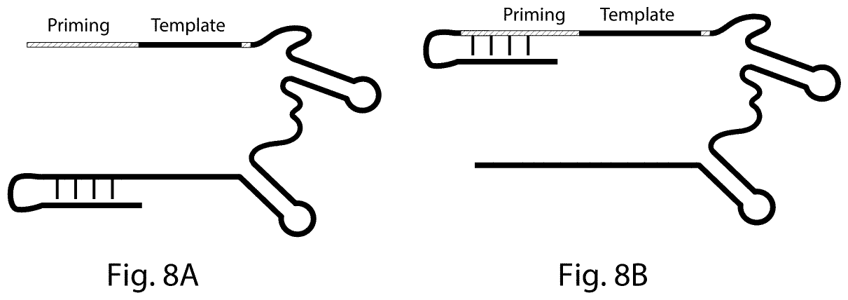

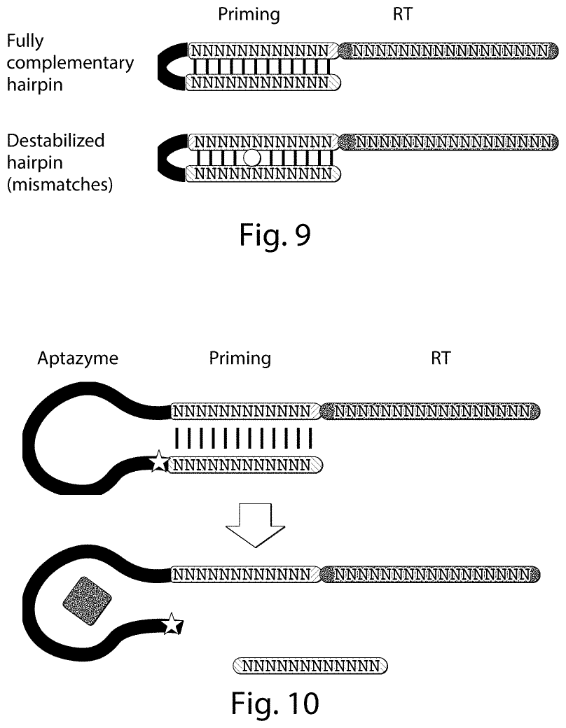

In some embodiments, a template nucleic acid (e.g., template RNA) as described herein further comprises a self-annealing region capable of hybridizing to another region of the template nucleic acid (see, e.g., FIG. 8). In some embodiments, the template nucleic acid comprises a self-annealing region capable of hybridizing to the sequence that binds to a target site, or a portion thereof, e.g., having a length of about 2-5, 5-10, 10-15, or 15-20 nucleotides (see, e.g., FIG. 8A). In certain embodiments, the self-annealing region comprises the reverse complement of the sequence that binds to a target site, or the portion thereof, or a sequence having at least 75%, 80%, 85%, 90%, 95%, 96%, 97%, 98%, or 99% sequence identity thereto. In certain embodiments, the self-annealing region comprises the reverse complement of the sequence that binds to a target site, or the portion thereof, or a sequence having no more than 1, 2, 3, 4, 5, 6, 7, 8, 9 or 10 mismatches relative thereto (see, e.g., FIG. 9). In certain embodiments, the self-annealing region is located at the 5′ end of the template nucleic acid (e.g., forms a hairpin at the 5′ end of the template nucleic acid).

In some embodiments, the template nucleic acid comprises a self-annealing region capable of hybridizing to the 3′ target homology domain, or a portion thereof, e.g., having a length of about 2-5, 5-10, 10-15, or 15-20 nucleotides (see, e.g., FIG. 8B). In certain embodiments, the self-annealing region comprises the reverse complement of the 3′ target homology domain, or the portion thereof, or a sequence having at least 75%, 80%, 85%, 90%, 95%, 96%, 97%, 98%, or 99% sequence identity thereto. In certain embodiments, the self-annealing region comprises the reverse complement of the 3′ target homology domain, or the portion thereof, or a sequence having no more than 1, 2, 3, 4, 5, 6, 7, 8, 9, or 10 mismatches relative thereto (see, e.g., FIG. 9). In certain embodiments, the self-annealing region is located at the 3′ end of the template nucleic acid (e.g., forms a hairpin at the 3′ end of the template nucleic acid).

In some embodiments, the self-annealing region, when hybridized to the other region of the template nucleic acid, forms a hairpin. In some embodiments, the hairpin has a stability of at least about −1, −2, −3, −4, −5, −6, −7, −8, −9, −10, −11, −12, −13, −14, −15, −16, −17, −18, −19, or −20 kcal/mol. In embodiments, the hairpin has a stability of at least about −10 kcal/mol. In some embodiments, the stability of the hairpin is lower than the stability of hybridization between the other region of the template nucleic acid and a target DNA sequence. In some embodiments, the stability of the hairpin is lower than, higher than, or equal to the stability of hybridization between sequence that binds to a target site and the 3′ target homology domain. In some embodiments, when the self-annealing region is hybridized to the other region of the template nucleic acid, the hybridization of that region to one or more other portions of the template nucleic acid is reduced, e.g., by at least 50%, 60%, 70%, 75%, 80%, 85%, 90%, 95%, 96%, 97%, 98%, 99%, or 100%. In certain embodiments, when the self-annealing region is hybridized to the sequence that binds a target site (or the portion thereof), the hybridization of the sequence that binds to a target site to a 3′ target homology domain of the template nucleic acid, or a portion thereof, is reduced, e.g., by at least 50%, 60%, 70%, 75%, 80%, 85%, 90%, 95%, 96%, 97%, 98%, 99%, or 100%. In certain embodiments, when the self-annealing region is hybridized to the 3′ target homology domain (or the portion thereof), the hybridization of the sequence that binds to a target site to a sequence that binds to a target site of the template nucleic acid, or a portion thereof, is reduced, e.g., by at least 50%, 60%, 70%, 75%, 80%, 85%, 90%, 95%, 96%, 97%, 98%, 99%, or 100%.

In some embodiments, the self-annealing region can be cleaved from the template nucleic acid molecule. In certain embodiments, the template nucleic acid molecule comprises (e.g., in or overlapping with the self-annealing region) a ribozyme capable of cleaving the template nucleic acid molecule to excise the self-annealing region. In embodiments, the ribozyme comprises a hammerhead ribozyme or an HDV ribozyme. In certain embodiments, the template nucleic acid molecule comprises (e.g., in or overlapping with the self-annealing region) an aptamer capable of binding to a ligand (e.g., a small molecule). In certain embodiments, the template nucleic acid molecule comprises (e.g., in or overlapping with the self-annealing region) an aptazyme capable of cleaving the template nucleic acid molecule to excise the self-annealing region when bound to a ligand (e.g., a small molecule) (see, e.g., FIG. 10). In embodiments, the ligand comprises theophylline, guanine, or a variant or derivative thereof. In certain embodiments, the template nucleic acid comprises (e.g., in or overlapping with the self-annealing region) a photo-cleavable region, e.g., wherein the self-annealing region is cleaved from the remainder of the template nucleic acid upon exposure to a particular wavelength of light.

Circular RNAs

It is contemplated that it may be useful to employ circular and/or linear RNA states during the formulation, delivery, or editing reaction within the target cell. Circular RNAs (circRNA) have been found to occur naturally in cells and have been found to have diverse functions, including both non-coding and protein coding roles in human cells. It has been shown that a circRNA can be engineered by incorporating a self-splicing intron into an RNA molecule (or DNA encoding the RNA molecule) that results in circularization of the RNA, and that an engineered circRNA can have enhanced protein production and stability (Wesselhoeft et al. Nature Communications 2018). In some embodiments, the polypeptide is encoded as circRNA. In some embodiments of any of the aspects described herein, a system comprises one or more circular RNAs (circRNAs). In some embodiments of any of the aspects described herein, a system comprises one or more linear RNAs. In some embodiments, a nucleic acid as described herein (e.g., a nucleic acid molecule encoding a polypeptide as described herein, or both) is a circRNA. In some embodiments, a circular RNA molecule encodes the polypeptide. In some embodiments, the circRNA molecule encoding the polypeptide is delivered to a host cell. In some embodiments, a circular RNA molecule encodes a recombinase, e.g., as described herein. In some embodiments, the circRNA molecule encoding the recombinase is delivered to a host cell. In some embodiments, the circRNA molecule encoding the polypeptide is linearized (e.g., in the host cell) prior to translation.

In some embodiments, the circRNA molecule (e.g., a template RNA molecule as described herein) comprises (e.g., from 5′ to 3′) (i) optionally a sequence that binds a target site in the DNA (e.g., a second strand of a site in a target genome), (ii) a sequence that binds the polypeptide, (iii) a heterologous object sequence, and (iv) optionally a 3′ target homology domain. In some embodiments, (i) and (iv) are linked, e.g., linked directly adjacent to each other, or linked by a linker region (e.g., a region having a length of about 1, 2, 3, 4, 5, 6, 7, 8, 9, 10, 20, 30, 40, 50, 60, 70, 80, 90, 100, 200, 300, 400, 500, or more nucleotides).

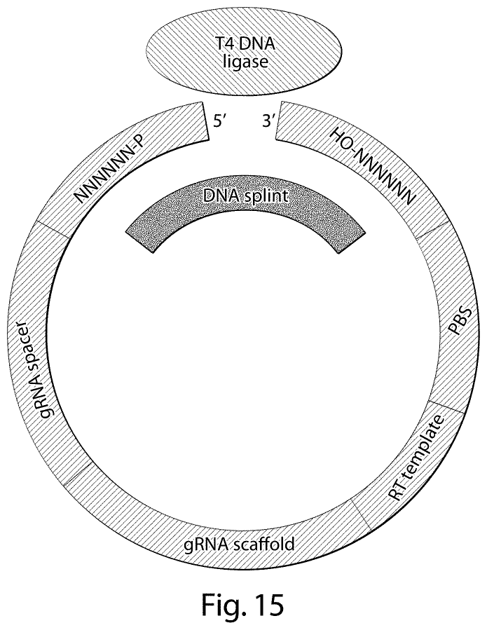

In some embodiments, the template RNA is circular. In some embodiments, the circular template RNA is made by a process using splint ligation. The process may comprise providing a splint oligonucleotide that has a first region of complementarity to the 3′ end of the linear template RNA and a second region of complementarity to the 5′ end of the linear template RNA. The splint oligonucleotide can be contacted with the linear template RNA under conditions that allow for annealing. A ligase (e.g., T4 DNA ligase) can then be added under conditions that allow for covalent linkage of the 5′ and 3′ ends. In some embodiments, the splint oligonucleotide comprises DNA. Such a system is illustrated in FIG. 15.

Circular RNAs (circRNAs) have been found to occur naturally in cells and have been found to have diverse functions, including both non-coding and protein coding roles in human cells. It has been shown that a circRNA can be engineered by incorporating a self-splicing intron into an RNA molecule (or DNA encoding the RNA molecule) that results in circularization of the RNA, and that an engineered circRNA can have enhanced protein production and stability (Wesselhoeft et al. Nature Communications 2018). In some embodiments, the polypeptide is encoded as circRNA. In certain embodiments, the template nucleic acid is a DNA, such as a dsDNA or ssDNA.

In some embodiments, the circRNA comprises one or more ribozyme sequences. In some embodiments, the ribozyme sequence is activated for autocleavage, e.g., in a host cell, e.g., thereby resulting in linearization of the circRNA. In some embodiments, the ribozyme is activated when the concentration of magnesium reaches a sufficient level for cleavage, e.g., in a host cell. In some embodiments the circRNA is maintained in a low magnesium environment prior to delivery to the host cell. In some embodiments, the ribozyme is a protein-responsive ribozyme. In some embodiments, the ribozyme is a nucleic acid-responsive ribozyme.

In some embodiments, the circRNA is linearized in the nucleus of a target cell. In some embodiments, linearization of a circRNA in the nucleus of a cell involves components present in the nucleus of the cell, e.g., to activate a cleavage event. For example, the B2 and ALU retrotransposons contain self-cleaving ribozymes whose activity is enhanced by interaction with the Polycomb protein, EZH2 (Hernandez et al. PNAS 117(1):415-425 (2020)). Thus, in some embodiments, a ribozyme, e.g., a ribozyme from a B2 or ALU element, that is responsive to a nuclear element, e.g., a nuclear protein, e.g., a genome-interacting protein, e.g., an epigenetic modifier, e.g., EZH2, is incorporated into a circRNA, e.g., of a system as described herein. In some embodiments, nuclear localization of the circRNA results in an increase in autocatalytic activity of the ribozyme and linearization of the circRNA.

In some embodiments, an inducible ribozyme (e.g., in a circRNA as described herein) is created synthetically, for example, by utilizing a protein ligand-responsive aptamer design. A system for utilizing the satellite RNA of tobacco ringspot virus hammerhead ribozyme with an MS2 coat protein aptamer has been described (Kennedy et al. Nucleic Acids Res 42(19):12306-12321 (2014), incorporated herein by reference in its entirety) that results in activation of the ribozyme activity in the presence of the MS2 coat protein. In embodiments, such a system responds to protein ligand localized to the cytoplasm or the nucleus. In some embodiments the protein ligand is not MS2. Methods for generating RNA aptamers to target ligands have been described, for example, based on the systematic evolution of ligands by exponential enrichment (SELEX) (Tuerk and Gold, Science 249(4968):505-510 (1990); Ellington and Szostak, Nature 346(6287):818-822 (1990); the methods of each of which are incorporated herein by reference) and have, in some instances, been aided by in silico design (Bell et al. PNAS 117(15):8486-8493, the methods of which are incorporated herein by reference). Thus, in some embodiments, an aptamer for a target ligand is generated and incorporated into a synthetic ribozyme system, e.g., to trigger ribozyme-mediated cleavage and circRNA linearization, e.g., in the presence of the protein ligand. In some embodiments, circRNA linearization is triggered in the cytoplasm, e.g., using an aptamer that associates with a ligand in the cytoplasm. In some embodiments, circRNA linearization is triggered in the nucleus, e.g., using an aptamer that associates with a ligand in the nucleus. In embodiments, the ligand in the nucleus comprises an epigenetic modifier or a transcription factor. In some embodiments the ligand that triggers linearization is present at higher levels in on-target cells than off-target cells.

It is further contemplated that a nucleic acid-responsive ribozyme system can be employed for circRNA linearization. For example, biosensors that sense defined target nucleic acid molecules to trigger ribozyme activation are described, e.g., in Penchovsky (Biotechnology Advances 32(5):1015-1027 (2014), incorporated herein by reference). By these methods, a ribozyme naturally folds into an inactive state and is only activated in the presence of a defined target nucleic acid molecule (e.g., an RNA molecule). In some embodiments, a circRNA of a system as described herein comprises a nucleic acid-responsive ribozyme that is activated in the presence of a defined target nucleic acid, e.g., an RNA, e.g., an mRNA, miRNA, guide RNA, gRNA, sgRNA, ncRNA, lncRNA, tRNA, snRNA, or mtRNA. In some embodiments the nucleic acid that triggers linearization is present at higher levels in on-target cells than off-target cells.

In some embodiments of any of the aspects herein, a system incorporates one or more ribozymes with inducible specificity to a target tissue or target cell of interest, e.g., a ribozyme that is activated by a ligand or nucleic acid present at higher levels in a target tissue or target cell of interest. In some embodiments, the system incorporates a ribozyme with inducible specificity to a subcellular compartment, e.g., the nucleus, nucleolus, cytoplasm, or mitochondria. In some embodiments, the ribozyme that is activated by a ligand or nucleic acid present at higher levels in the target subcellular compartment. In some embodiments, an RNA component of a system is provided as circRNA, e.g., that is activated by linearization. In some embodiments, linearization of a circRNA encoding a polypeptide activates the molecule for translation. In some embodiments, a signal that activates a circRNA component of a system is present at higher levels in on-target cells or tissues, e.g., such that the system is specifically activated in these cells.

In some embodiments, an RNA component of a system is provided as a circRNA that is inactivated by linearization. In some embodiments, a circRNA encoding the polypeptide is inactivated by cleavage and degradation. In some embodiments, a circRNA encoding the polypeptide is inactivated by cleavage that separates a translation signal from the coding sequence of the polypeptide. In some embodiments, a signal that inactivates a circRNA component of a system is present at higher levels in off-target cells or tissues, such that the system is specifically inactivated in these cells.

In some embodiments, one or more circRNA molecules of a system as described herein comprises a nuclear localization signal.

Functional Characteristics

In some embodiments, a template nucleic acid, e.g., template RNA, is a substrate for target-primed reverse transcription, e.g., by a polypeptide, e.g., a genome editing polypeptide as described herein. In some embodiments, the heterologous object sequence is a substrate for target-primed reverse transcription, e.g., by a polypeptide, e.g., a genome editing polypeptide as described herein.

In some embodiments, the heterologous object sequence of a template nucleic acid (e.g., template RNA) does not comprise self-complementary sequences, e.g., that form hairpin structures, e.g., under stringent conditions. In some embodiments, if a set of self-complementary sequences is present in the heterologous object sequence, each self-complementary sequence is no more than 10, 9, 8, 7, 6, 5, 4, or 3 nucleotides in length. In some embodiments, if a set of self-complementary sequences is present in the heterologous object sequence, the self-complementary sequence forms a hairpin comprising arms of no longer than 10, 9, 8, 7, 6, 5, 4, or 3 nucleotides in length. In some embodiments, if a set of self-complementary sequences is present in the heterologous object sequence, the self-complementary sequence comprises at least 1, 2, 3, 4, or 5 positions of non-complementarity (e.g., mismatches or bulges) with its partner sequence. In some embodiments, the heterologous object sequence of a template nucleic acid (e.g., template RNA) does not comprise a repetitive sequence (e.g., a single-, di-, or tri-nucleotide repetitive sequence) or if a repetitive sequence is present it is of no more than 12, 11, 10, 9, 8, 7, or 6 nucleotides in length.

In some embodiments, the processivity of a polypeptide, e.g., a genome editing polypeptide as described herein, reverse transcribing the heterologous object sequence is increased relative to the processivity of a polypeptide, e.g., a genome editing polypeptide as described herein, reverse transcribing a sequence similar to the heterologous object sequence but not comprising the alteration. In some embodiments, the processivity of a polypeptide, e.g., a genome editing polypeptide as described herein, reverse transcribing the heterologous object sequence is at least 1, 2, 3, 4, 5, 6, 7, 8, 9, 10, 20, 30, 40, 50, 60, 70, 80, 90, 100, 120, 140, 160, 180, 200, 300, 400, 500, or 1000% increased relative to the processivity of a polypeptide, e.g., a genome editing polypeptide as described herein, reverse transcribing a sequence similar to the heterologous object sequence but not comprising the alteration.

In some embodiments, the polymerization rate of a polypeptide, e.g., a genome editing polypeptide as described herein, reverse transcribing the heterologous object sequence is increased relative to the polymerization rate of a polypeptide, e.g., a genome editing polypeptide as described herein, reverse transcribing a sequence similar to the heterologous object sequence but not comprising the alteration. In some embodiments, the polymerization rate of a polypeptide, e.g., a genome editing polypeptide as described herein, reverse transcribing the heterologous object sequence is at least 1, 2, 3, 4, 5, 6, 7, 8, 9, 10, 20, 30, 40, 50, 60, 70, 80, 90, 100, 120, 140, 160, 180, 200, 300, 400, 500, or 1000% increased relative to the polymerization rate of a polypeptide, e.g., a genome editing polypeptide as described herein, reverse transcribing a sequence similar to the heterologous object sequence but not comprising the alteration.

In some embodiments, the error rate of a polypeptide, e.g., a genome editing polypeptide as described herein, reverse transcribing the heterologous object sequence is decreased relative to the error rate a polypeptide, e.g., a genome editing polypeptide as described herein, reverse transcribing a sequence similar to the heterologous object sequence but not comprising the alteration (e.g., as measured by RT fidelity assay, or Next Generation Sequencing (NGS)). In some embodiments, the error rate of a polypeptide, e.g., a genome editing polypeptide as described herein, reverse transcribing the heterologous object sequence is at least 1, 2, 3, 4, 5, 6, 7, 8, 9, 10, 20, 30, 40, 50, 60, 70, 80, 90, or 100% decreased relative to the error rate of a polypeptide, e.g., a genome editing polypeptide as described herein, reverse transcribing a sequence similar to the heterologous object sequence but not comprising the alteration.

In some embodiments, the template RNA has minimal energy structures between −280 and −480 kcal/mol (e.g., between −280 and −300, −300 and −320, −320 and −340, −340 and −360, −360 and −380, −380 and −400, −400 and −420, −420 and −440, −440 and −460, −460 and −480 kcal/mol). In embodiments, the energy structures of the template RNA are measured by RNAstructure, e.g., as described in Turner and Mathews (2009) Nucleic Acids Res 38:D280-282 (incorporated by reference herein in its entirety).

In some embodiments, the template RNA is sequence optimized, e.g., to reduce secondary structures, for example, as measured by RNAstructure per the methods of Turner and Mathews (2009) Nucleic Acids Res 38:D280-282. In some embodiments, the template RNA is sequence optimized, e.g., to reduce secondary structure, for example, as measured in vitro by SHAPE-MaP, e.g., as described in Siegfried et al. Nat Methods (2014) 11:959-965 (incorporation by reference herein in its entirety). In some embodiments, the template RNA is sequence optimized, e.g., to reduce secondary structure, for example, as measured in cells by DMS-MaPseq, e.g., as described in Zubradt et al. Nat Methods (2017) 14:75-82 (incorporated by reference herein in its entirety).

Chemically Modified Nucleic Acids and Nucleic Acid End Features

A nucleic acid described herein (e.g., a template nucleic acid, e.g., a template RNA; or a nucleic acid (e.g., mRNA) encoding a polypeptide as described herein; or a gRNA) can comprise unmodified or modified nucleobases. Naturally occurring RNAs are synthesized from four basic ribonucleotides: ATP, CTP, UTP and GTP, but may contain post-transcriptionally modified nucleotides. Further, approximately one hundred different nucleoside modifications have been identified in RNA (Rozenski, J, Crain, P, and McCloskey, J. (1999). The RNA Modification Database: 1999 update. Nucl Acids Res 27: 196-197). An RNA can also comprise wholly synthetic nucleotides that do not occur in nature.

In some embodiments, a chemically modified nucleotide in the template, upon reverse transcription, directs insertion of a canonical nucleotide into the genome. In some embodiments, the canonical nucleotide is the corresponding nucleotide of the original sequence. For instance, a pseudouridine in the heterologous object sequence would be written into the genome as an adenine in the reverse transcribed strand, which would base pair with a thymine in the other DNA strand. Thus, in some embodiments, a template RNA described herein comprises a one or more nucleotide that is chemically modified relative to a corresponding original sequence, but insertion of the heterologous object sequence into the genome results in presence of the corresponding nucleotide of the original sequence in the genome.

In some embodiments, the template RNA comprises at least 1, 2, 3, 4, 5, 10, 20, 50, 100, 200, or 500 modified nucleotides. In some embodiments, all nucleotides of the template RNA are modified. In some embodiments, all adenines of the template RNA are chemically modified. In some embodiments, all cytosines of the template RNA are chemically modified. In some embodiments, all guanines of the template RNA are chemically modified. In some embodiments, all uracils of the template RNA are chemically modified.

Without wishing to be bound by theory, in some embodiments, the chemical modification reduces secondary structure of the template RNA. More specifically, in some embodiments, the heterologous object sequence and the sequence that binds a target site in the DNA have complementary regions and can pair with each other, leading to reduced ability of the protein-RNA complex to bind a target site. Alternatively or in combination, the heterologous object sequence may comprise self-complementary regions that impair reverse transcriptase activity.

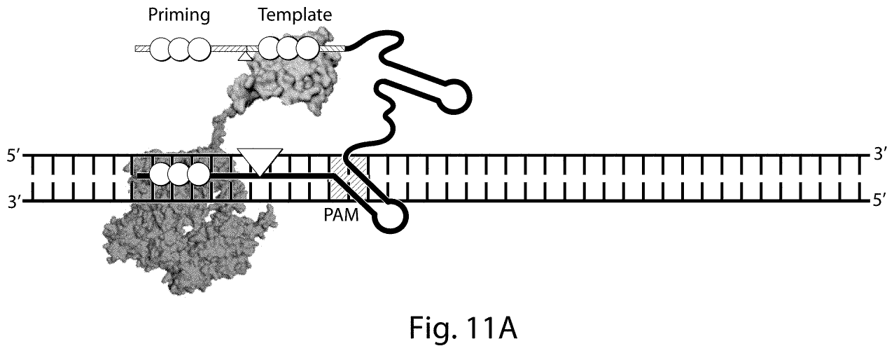

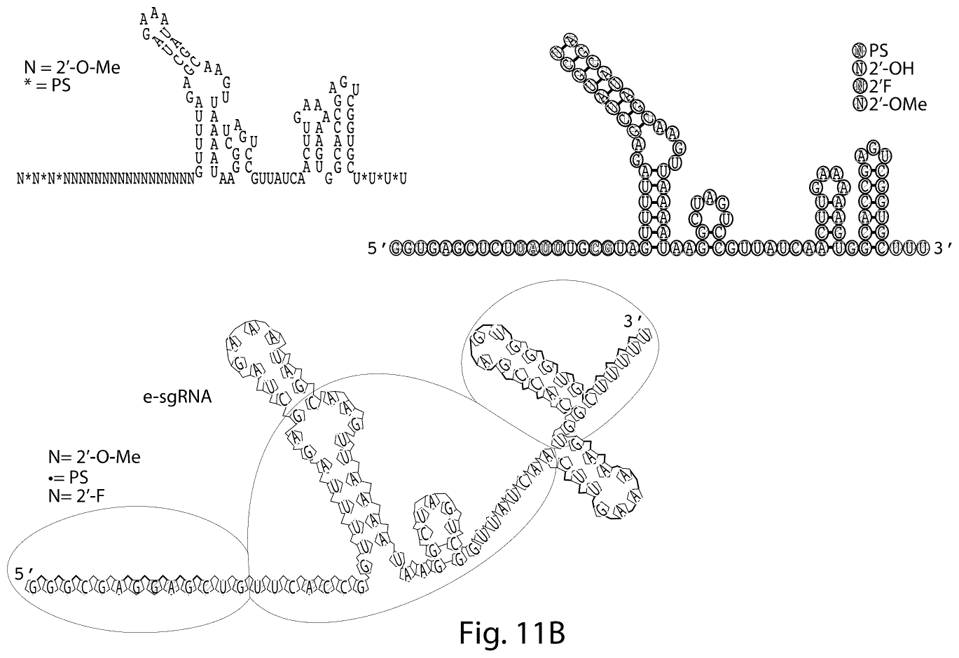

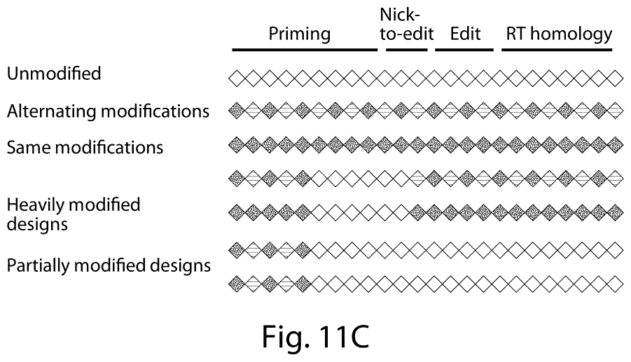

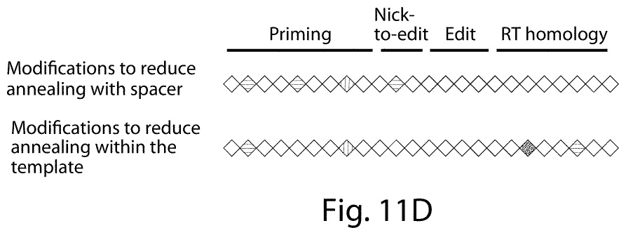

According to the teachings of the present disclosure, chemical modifications can be positioned at self-complementary sites in the target RNA to reduce undesired secondary structure. Exemplary positions for chemical modifications are shown in FIG. 11. The template RNA may comprise (from 5′ to 3′) (i) a sequence that binds a target site in the DNA, (ii) a sequence that binds the polypeptide comprising an RT domain and/or endonuclease domain, (iii) a heterologous object sequence, and (iv) a 3′ target homology domain. In some embodiments, one or more chemical modifications are situated in (iv) the 3′ target homology domain, e.g., in a region of (iv) that has complementarity with a second region in the template RNA, e.g., wherein the second region is a region in (i) the sequence that binds a target site in the DNA. In some embodiments, one or more chemical modifications are situated in (i) the sequence that binds a target site in the DNA, e.g., in a region of (i) that has complementarity with a second region in the template RNA, e.g., wherein the second region is a region in (iv) the 3′ target homology domain. In some embodiments, one or more chemical modifications are situated in (iii) the heterologous object sequence, e.g., in a region of the heterologous object sequence that has self-complementarity with a second region of the template RNA, e.g., a second region within the heterologous object sequence.

In some embodiments, the one or more chemical modifications comprise 1, 2, 3, 4, 5, 6, 7, 8, or 10 chemical modifications. In some embodiments, the chemical modification is one that destabilizes RNA:RNA base pairing. In some embodiments, one or more chemically modified nucleotide can be situated to disrupt 1, 2, or 3 base pairing interactions in a double stranded region (e.g., a stem loop). For instance, in some embodiments, a first chemically modified nucleotide disrupts a first base pair of the double stranded region, a second chemically modified nucleotide disrupts a second base pair of the double stranded region, and optionally a third chemically modified nucleotide disrupts a third base pair of the double stranded region. In some embodiments, the chemical modification is in a backbone or a base. In some embodiments, the one or more chemically modified nucleotides comprise an unlocked nucleic acid (UNA), Glycol nucleic acid (GNA), or an N1-methyl nucleotide (e.g., N1-methyl deoxyadenosine, N1-methyl deoxyguanosine, N1-methyl adenosine, or N1-methyl guanosine) (see Zhou et al., 2021 doi: 10.1038/nsmb.3270). In some embodiments, the one or more chemically modified nucleotide comprises a 2′-O-methoxy-ethyl nucleotide.

In some embodiments, the alteration (e.g., chemical modification or mutation) increases the free energy of the interaction between the two self-complementary sequences in the template RNA. For example, the free energy may be increased by about 0.5-2, 2-5, 5-10, 10-15, 15-20, 20-25, or 25-30 kcal/mol. In some embodiments, the free energy of the interaction between the sequences comprising the alteration is at least about −25, −20, −15, −10, −5, −3, or −2 kcal/mol. In some embodiments, the alteration is a mutation comprising 1, 2, 3, or 4 differences compared to the corresponding sequence of the target DNA.

In some embodiments, chemical modifications are situated on (i) the sequence that binds a target site in the DNA or (ii) the sequence that binds the polypeptide comprising an RT domain and/or endonuclease domain. In some embodiments, the structure of (ii) comprises (e.g., as shown from left to right in FIG. 11B, top left panel), a lower stem/bulge/upper stem, a nexus, a Hairpin 1, and a Hairpin 2 (see Briner, et al. (2014). Guide RNA Functional Modules Direct Cas9 Activity and Orthogonality. Molecular Cell, 56, 333-339; DOI: 10.1016/j.molcel.2014.09.019)

In some embodiments, the chemical modifications (e.g., 2′-O-Me) are situated in one or more of (e.g., two or all of) the upper stem and the loop of the upper stem; hairpin 1; and hairpin 2. In some embodiments, the template RNA comprises one or more (e.g., 2, 3, 4, or 5) backbone modifications (e.g., phosphorothioate modifications) at the 5′ end of the template RNA. In some embodiments, the template RNA comprises one or more (e.g., 2, 3, 4, or 5) backbone modifications (e.g., phosphorothioate modifications) at the 3′ end of the template RNA (e.g., at the 3′ of (iv)). In some embodiments, the chemical modifications are at one or more (e.g., all) positions shown in FIG. 11B, top left panel.

In some embodiments, the chemical modifications (e.g., 2′-O-Me) are situated in one or more of (e.g., all of) the lower stem, bulge, upper stem, and the loop of the upper stem; the nexus; hairpin 1; and hairpin 2. In some embodiments, chemical modifications (e.g., 2′F) are situated in (i) the sequence that binds a target site in the DNA. In some embodiments, the template RNA comprises one or more (e.g., 2, 3, 4, or 5) backbone modifications (e.g., phosphorothioate modifications) at one or more of (e.g., all of) the following positions: the 5′ end of the template RNA, in (i) the sequence that binds a target site in the DNA, or in (ii) (e.g., in hairpin 1 and/or hairpin 2 therein). In some embodiments, the chemical modifications are at one or more (e.g., all) positions shown in FIG. 11B, bottom left panel.

In some embodiments, the chemical modifications (e.g., 2′-O-Me) are situated in one or more of (e.g., all of) (i) the sequence that binds a target site in the DNA, lower stem, bulge, upper stem, and the loop of the upper stem; the nexus; hairpin 1; and hairpin 2. In some embodiments, chemical modifications (e.g., 2′F) are situated in one or both of (i) the sequence that binds a target site in the DNA, and (ii) (e.g., in the lower stem therein). In some embodiments, the template RNA comprises one or more (e.g., 2, 3, 4, or 5) backbone modifications (e.g., phosphorothioate modifications) at one or more of (e.g., all of) the following positions: the 5′ end of the template RNA, in (i) the sequence that binds a target site in the DNA, or in (ii) (e.g., in the lower stem and/or the upper stem therein). In some embodiments, the chemical modifications are at one or more (e.g., all) positions shown in FIG. 11B, right panel.

Additional exemplary patterns of modification are shown in FIG. 11C. In some embodiments, a template RNA described herein comprises one or more chemical modifications at one or more (e.g., all) positions shown in FIG. 11C. For instance, in some embodiments, the template RNA comprises the same chemical modification at all positions in (iii) the heterologous object sequence and (iv) the 3′ target homology domain. In some embodiments, the template RNA comprises alternating chemical modification at all positions in (iii) the heterologous object sequence and (iv) the 3′ target homology domain. In some embodiments, the template RNA comprises chemical modifications (e.g., the same modification or alternating modifications) at the 3′ end of (iv). In some embodiments, the template RNA comprises chemical modifications (e.g., the same modification or alternating modifications) at the 5′ end of the nick-to-edit region (see FIG. 11C legend), in the edit region, and in the RT homology region.

Additional exemplary patterns of modification are shown in FIG. 11D. In some embodiments, a template RNA described herein comprises one or more chemical modifications at one or more (e.g., all) positions shown in FIG. 11D. For instance, in some embodiments, the template RNA comprises chemical modifications in (iv) the 3′ target homology domain and in (iii) the heterologous object sequence (e.g., within the nick-to-edit region therein). Without wishing to be bound by theory, in some embodiments these chemical modifications reduce annealing between the (i) a sequence that binds a target site in the DNA and the (iv)/(iii) regions. Alternatively or in combination, in some embodiments, the template RNA comprises chemical modifications in (iv) the 3′ target homology domain and in (iii) the heterologous object sequence (e.g., within the RT homology domain therein). Without wishing to be bound by theory, in some embodiments these chemical modifications reduce annealing between the (iv) region and the (iii) region.

Exemplary chemical modifications suitable for use in a template RNA described herein (e.g., to disrupt secondary structure) include 2′-MOE, 2′-OMe, 2′-F, 2′-FANA, 2′-deoxy, PNA, LNA, UNA, GNA, PS, N1-methyl deoxyadenosine, N1-methyl deoxyguanosine, N1-methyl adenosine, N1-methyl guanosine. In some embodiments, the template RNA is free of chemical modifications to the 2′ OH. In some embodiments, one or both of regions (iii) and (iv) in the template RNA are free of chemical modifications to the 2′ OH.

In some embodiments, the chemical modification is one provided in PCT/US2016/032454, US Pat. Pub. No. 20090286852, of International Application No. WO/2012/019168, WO/2012/045075, WO/2012/135805, WO/2012/158736, WO/2013/039857, WO/2013/039861, WO/2013/052523, WO/2013/090648, WO/2013/096709, WO/2013/101690, WO/2013/106496, WO/2013/130161, WO/2013/151669, WO/2013/151736, WO/2013/151672, WO/2013/151664, WO/2013/151665, WO/2013/151668, WO/2013/151671, WO/2013/151667, WO/2013/151670, WO/2013/151666, WO/2013/151663, WO/2014/028429, WO/2014/081507, WO/2014/093924, WO/2014/093574, WO/2014/113089, WO/2014/144711, WO/2014/144767, WO/2014/144039, WO/2014/152540, WO/2014/152030, WO/2014/152031, WO/2014/152027, WO/2014/152211, WO/2014/158795, WO/2014/159813, WO/2014/164253, WO/2015/006747, WO/2015/034928, WO/2015/034925, WO/2015/038892, WO/2015/048744, WO/2015/051214, WO/2015/051173, WO/2015/051169, WO/2015/058069, WO/2015/085318, WO/2015/089511, WO/2015/105926, WO/2015/164674, WO/2015/196130, WO/2015/196128, WO/2015/196118, WO/2016/011226, WO/2016/011222, WO/2016/011306, WO/2016/014846, WO/2016/022914, WO/2016/036902, WO/2016/077125, or WO/2016/077123, each of which is herein incorporated by reference in its entirety. It is understood that incorporation of a chemically modified nucleotide into a polynucleotide can result in the modification being incorporated into a nucleobase, the backbone, or both, depending on the location of the modification in the nucleotide. In some embodiments, the backbone modification is one provided in EP 2813570, which is herein incorporated by reference in its entirety. In some embodiments, the modified cap is one provided in US Pat. Pub. No. 20050287539, which is herein incorporated by reference in its entirety.

In some embodiments, the chemically modified nucleic acid (e.g., RNA, e.g., mRNA) comprises one or more of ARCA: anti-reverse cap analog (m27.3′-OGP3G), GP3G (Unmethylated Cap Analog), m7GP3G (Monomethylated Cap Analog), m32.2.7GP3G (Trimethylated Cap Analog), m5CTP (5′-methyl-cytidine triphosphate), m6ATP (N6-methyl-adenosine-5′-triphosphate), s2UTP (2-thio-uridine triphosphate), and Ψ (pseudouridine triphosphate).

In some embodiments, the chemically modified nucleic acid comprises a 5′ cap, e.g.: a 7-methylguanosine cap (e.g., a O-Me-m7G cap); a hypermethylated cap analog; an NAD+-derived cap analog (e.g., as described in Kiledjian, Trends in Cell Biology 28, 454-464 (2018)); or a modified, e.g., biotinylated, cap analog (e.g., as described in Bednarek et al., Phil Trans R Soc B 373, 20180167 (2018)).

In some embodiments, the chemically modified nucleic acid comprises a 3′ feature selected from one or more of: a polyA tail; a 16-nucleotide long stem-loop structure flanked by unpaired 5 nucleotides (e.g., as described by Mannironi et al., Nucleic Acid Research 17, 9113-9126 (1989)); a triple-helical structure (e.g., as described by Brown et al., PNAS 109, 19202-19207 (2012)); a tRNA, Y RNA, or vault RNA structure (e.g., as described by Labno et al., Biochemica et Biophysica Acta 1863, 3125-3147 (2016)); incorporation of one or more deoxyribonucleotide triphosphates (dNTPs), 2′O-Methylated NTPs, or phosphorothioate-NTPs; a single nucleotide chemical modification (e.g., oxidation of the 3′ terminal ribose to a reactive aldehyde followed by conjugation of the aldehyde-reactive modified nucleotide); or chemical ligation to another nucleic acid molecule.

In some embodiments, the nucleic acid (e.g., template nucleic acid) comprises one or more modified nucleotides, e.g., selected from dihydrouridine, inosine, 7-methylguanosine, 5-methylcytidine (5mC), 5′ Phosphate ribothymidine, 2′-O-methyl ribothymidine, 2′-O-ethyl ribothymidine, 2′-fluoro ribothymidine, C-5 propynyl-deoxycytidine (pdC), C-5 propynyl-deoxyuridine (pdU), C-5 propynyl-cytidine (pC), C-5 propynyl-uridine (pU), 5-methyl cytidine, 5-methyl uridine, 5-methyl deoxycytidine, 5-methyl deoxyuridine methoxy, 2,6-diaminopurine, 5′-Dimethoxytrityl-N4-ethyl-2′-deoxycytidine, C-5 propynyl-f-cytidine (pfC), C-5 propynyl-f-uridine (pfU), 5-methyl f-cytidine, 5-methyl f-uridine, C-5 propynyl-m-cytidine (pmC), C-5 propynyl-f-uridine (pmU), 5-methyl m-cytidine, 5-methyl m-uridine, LNA (locked nucleic acid), MGB (minor groove binder) pseudouridine (Ψ), 1-N-methylpseudouridine (1-Me-Ψ), or 5-methoxyuridine (5-MO-U).

In some embodiments, the nucleic acid comprises a backbone modification, e.g., a modification to a sugar or phosphate group in the backbone. In some embodiments, the nucleic acid comprises a nucleobase modification.

In some embodiments, the nucleic acid comprises a sugar modification, e.g., as listed in Table 1 below. In some embodiments, the sugar modifications of Table 1 are situated at the three 3′-most nucleotides and the three 5′-most nucleotides of the template RNA.

TABLE 1
Exemplary sugar modifications

| Modification | Structure | Impact |
|---|---|---|
| 2′-F RNA | 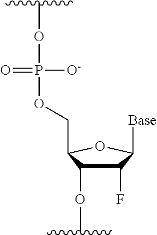 | Increases duplex T_{M} by stabilizing 3′ endo ribose conformation Increases nuclease resistance |
| 2′-OMe RNA | 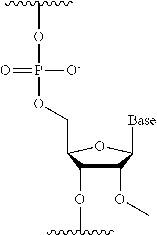 | Increases duplex T_{M} by stabilizing 3′ endo ribose conformation Increases nuclease resistance |
| 2′O-methoxyethyl (2′O- MOE) | 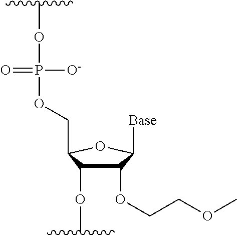 | Increases duplex T_{M} by stabilizing 3′ endo ribose conformation Increases nuclease resistance |
| 2′O-Allyl | 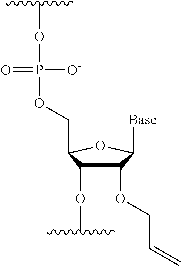 | Improved gene silencing of SiRNAs |
| 2′O-ethylamine (EA) | 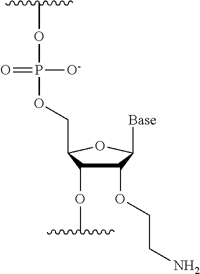 | Tolerated modification for gene silencing SiRNAs |
| 2′O-cyanoethyl | 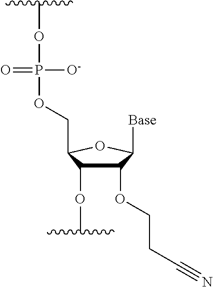 | Tolerated modification for gene silencing SIRNAs |
| 2′-FANA | 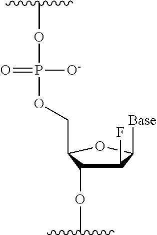 | Enhances binding to RNA Increased nuclease resistance |
| 4′S-RNA | 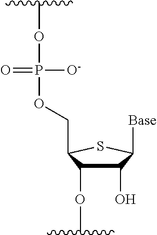 | Enhances nuclease stability |
| 4′C-RNA | 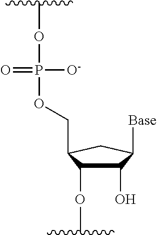 | Enhances nuclease stability |
| Locked Nucleic Acid (LNA) | 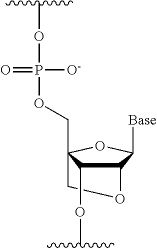 | Reduces conformational flexibility of nucleotides, C3′ endo conformation locked Increases nuclease resistance |
| Unlocked Nucleic Acids (UNA) | 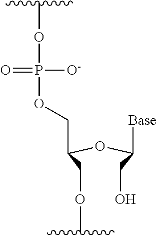 | Increases conformational flexibility of nucleotides Increases resistance from exoribonucleases |
| Glycol Nucleic Acid (GNA) | 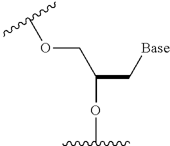 | Increases conformational flexibility of nucleotides Increases resistance from exoribonucleases Mitigates off-target effects in RNAi |
| Hexito Nucleic Acids (HNA) | 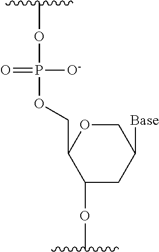 | Increases nuclease resistance in serum |
| Cyclohexenyl Nucelic Acid (CeNA) | 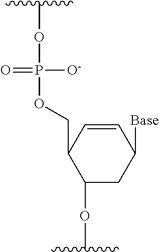 | Reduces conformational flexibility of nucleotides, stabilizes C3′ endo conformation Increases nuclease resistance in serum |
| | 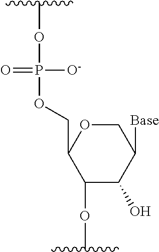 | Increases nuclease resistance in serum |
| 2′-deoxy- methanocarbanucleosides (MC) | 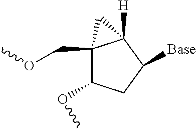 | Enhanced SiRNA serum stability Enhance thermal stability of duplex |
| Tricyclo-DNA (tc-DNA) | 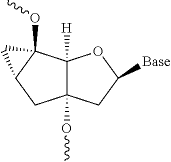 | Enhances binding affinity to RNA Stable to nucleases |
| 2'O-GalNAc | 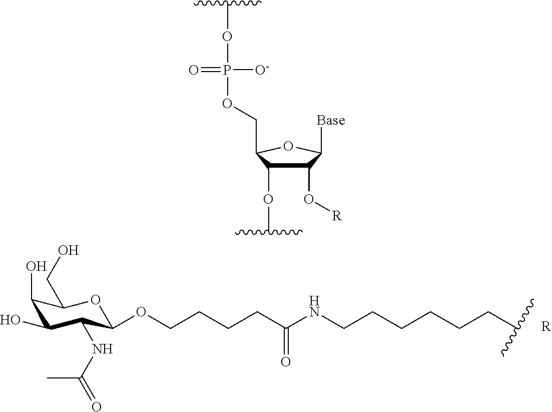 | Targeted delivery to hepatocytes |

In some embodiments, the nucleic acid comprises a backbone modification, e.g., as listed in Table 2 below. In some embodiments, backbone modifications of Table 2 are situated, at the three 3′-most internucleotide linkages and the three 5′-most internucleotide linkages of the template RNA.

TABLE 2
Exemplary phosphate backbone modifications

| Modification | Structure | Impact |
|---|---|---|
| Phophorothioate (PS) | 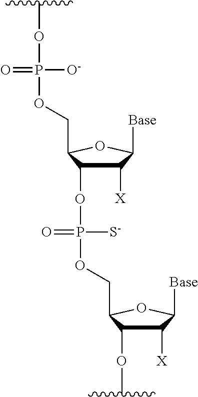 | Improved gene-silencing Enhances nuclease stability Extensive PS modification reduces silencing and has toxic side-effects |
| Boranophosphate (BP) | 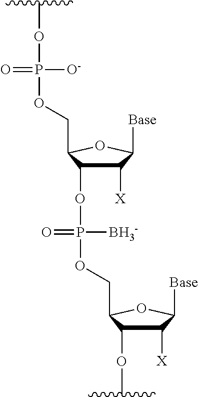 | Increased nuclease stability compared to PS Better tolerated in SiRNA design |
| 2,5-Linked | 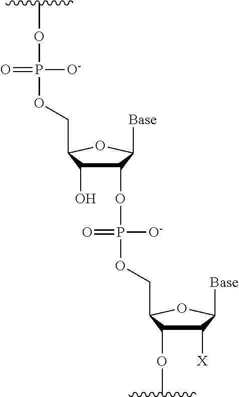 | Structurally similar to 3,5 linkage Decreased off-target effects in RNAi |
| Triazole-linked | 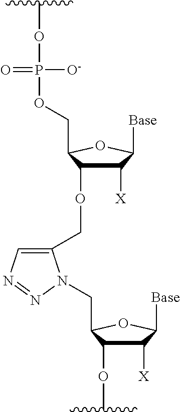 | Decreases duplex T_{M} Increases nuclease resistance |
| Peptide Nucleic Acid (PNA) | 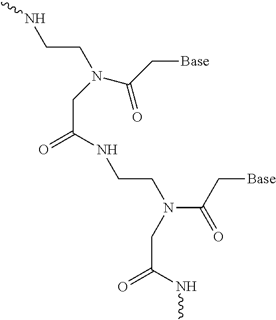 | Significant nuclease and protease resistance High affinity to DNA and RNA |
| Morpholino Phosphoramidate | 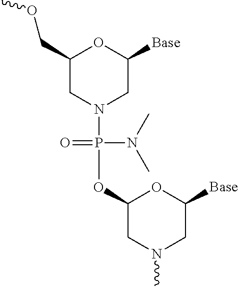 | Improved binding affinity to target strands Increases nuclease resistance |
| N3′ Phosphoramidate (NP) | 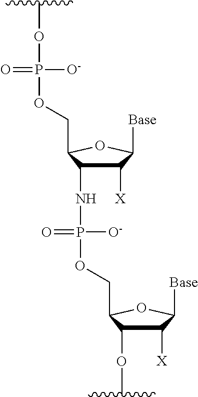 | Good binding affinity to RNA Increases resistance to nuclease degradation |
| Phosphonoacetate | 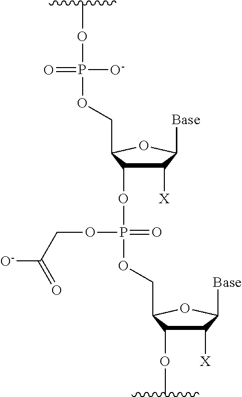 | Enhance nuclease stability Reduces gRNA off-target effects in conjuction with 2′-OMe |

In some embodiments, the nucleic acid comprises a protecting group (e.g., a sterically bulky protecting group), e.g., as listed in Table 3 below. In some embodiments, the template RNA comprises a bulky protecting group of Table 3 at its 5′ end. In some embodiments, the template RNA comprises a bulky protecting group of Table 3 at its 3′ end. In some embodiments, the template RNA comprises a bulky protecting group of Table 3 at each of its 5′ and 3′ ends. In some embodiments, a template RNA having a bulky protecting group described herein further comprises one or more additional chemical modifications. For instance, the template RNA may further comprise phosphorothioate linkages, e.g., at the three 3′-most internucleotide linkages and the three 5′-most internucleotide linkages of the template RNA. In some embodiments, the template RNA may further comprise a modification of Table 1 (e.g., a 2′-O-Me nucleotide) at the three 3′-most nucleotides and the three 5′-most nucleotides of the template RNA

TABLE 3
Exemplary 5′ and 3′ sterically bulky protecting groups

| Modification | Structure |
|---|---|
| 5′-Biotin | 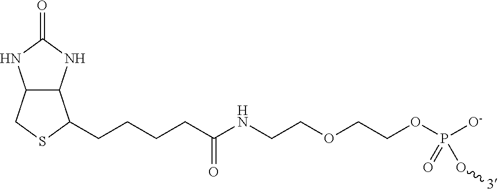 |
| 3′-Biotin | 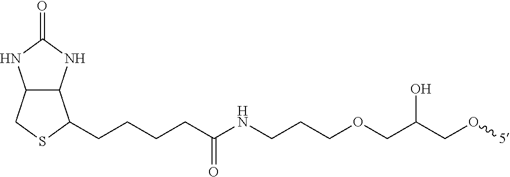 |
| 5′-Biotin- TEG | 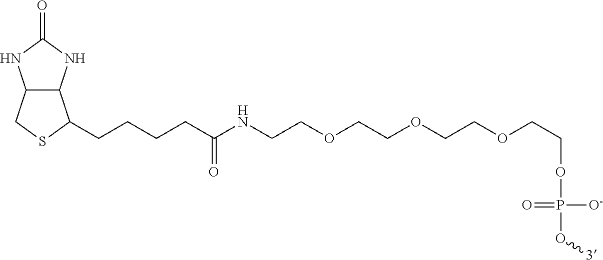 |
| 3′-Biotin- TEG | 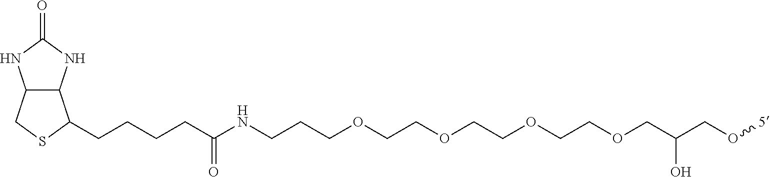 |
| 5′-Dual- Biotin | 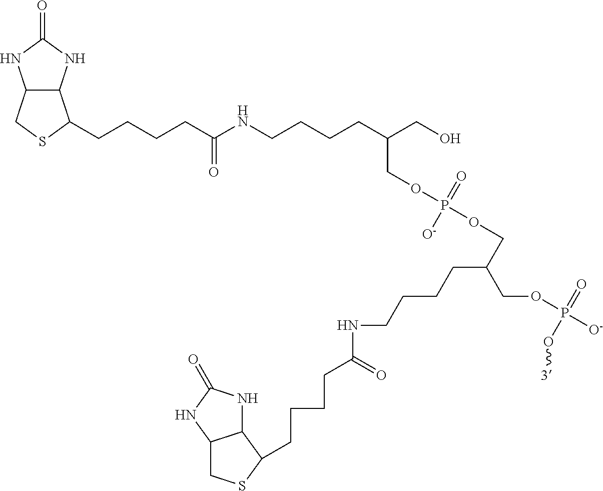 |
| Cholesterol- TEG | 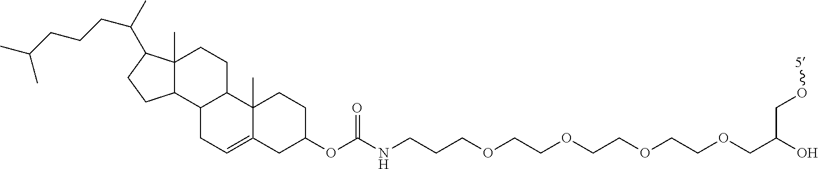 |

In some embodiments, the nucleic acid comprises an internal spacer region, e.g., as listed in Table 4 below. In some embodiments, an internal spacer of Table 4 is situated in the template RNA at a position that blocks the reverse transcriptase from reverse transcribing the region of the template RNA that binds the polypeptide. For instance, in some embodiments the internal spacer of Table 4 is situated immediately 3′ of (iii) the heterologous object sequence. In some embodiments, a template RNA having an internal spacer as described herein further comprises one or more additional chemical modifications. For instance, the template RNA may further comprise phosphorothioate linkages, e.g., at the three 3′-most internucleotide linkages and the three 5′-most internucleotide linkages of the template RNA. In some embodiments, the template RNA may further comprise a modification of Table 1 (e.g., a 2′-O-Me nucleotide) at the three 3′-most nucleotides and the three 5′-most nucleotides of the template RNA.

TABLE 4
Exemplary internal spacers

| | | Mode |
|---|---|---|
| | | of |
| Modifi- | | Attach- |
| cation | Structure | ment |
| triazole | 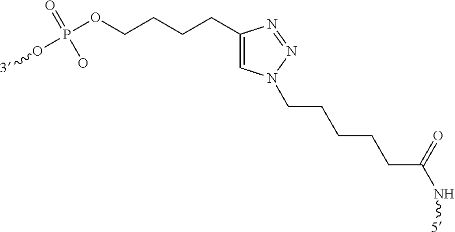 | Chem- ical reac- tion (Click) |
| DBCO- triazole | 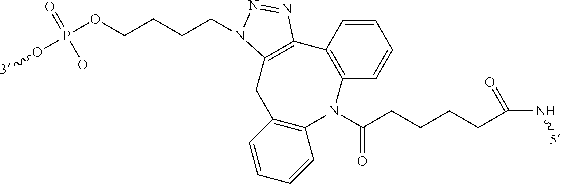 | Chem- ical reac- tion (Click) |
| DBCO- TEG- triazole | 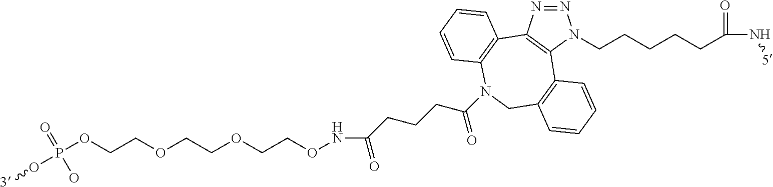 | Chem- ical reac- tion (Click) |
| Biotin- Strepta- vidin- Biotin | 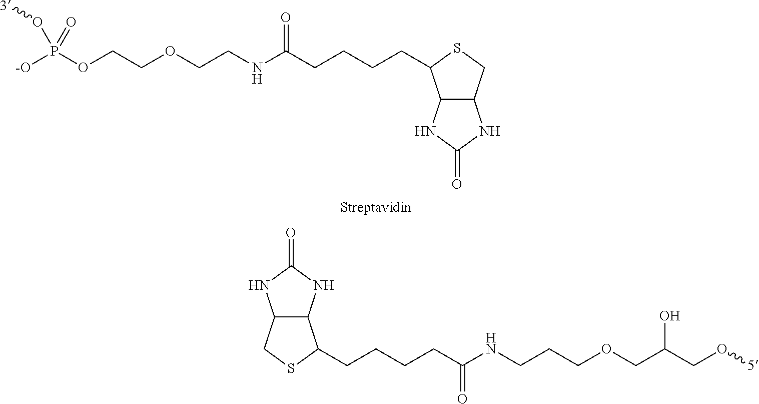 | Affin- ity |

In some embodiments, the nucleic acid comprises a nucleobase modification, e.g., as listed in Table 5 below.

TABLE 5
Exemplary nucleobase modifications

| Modification | Structure | Impact |
|---|---|---|
| 5-Methoxy-U | 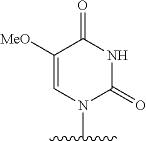 [SMILES: *n1cc(OC)c(=O)[nH]c1=O] | Improved immunogenicity profile of Cas9 mRNA Improves translational efficiency |
| Pseudo-U | 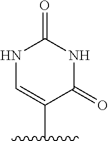 [SMILES: *c1c[nH]c(=O)[nH]c1=O] | Improved immunogenicity profile of Cas9 mRNA Improves translational efficiency |
| 5-Methyl-C | 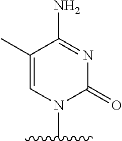 [SMILES: *n1cc(C)c(N)nc1=O] | Improved siRNA potency when used in conjuction with 2′- FANA and LNA |
| N1-Methyl-Pseudo-U | 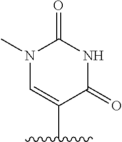 [SMILES: *c1cn(C)c(=O)[nH]c1=O] | Improved immunogenicity profile of Cas9 mRNA Improves translational efficiency |
| 5-Hydroxymethyl-U | 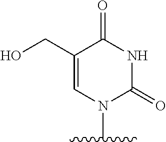 [SMILES: *n1cc(CO)c(=O)[nH]c1=O] | Improved immunogenicity profile of Cas9 mRNA Improves translational efficiency |
| 2-thio-U | 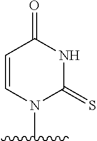 [SMILES: *n1ccc(=O)[nH]c1=S] | Increases specificity and potency in SiRNA Tolerated in mRNAs |
| 5-Bromo-U | 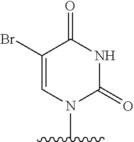 [SMILES: *n1cc(Br)c(=O)[nH]c1=O] | Stabilize A-U pairs, tolerated in siRNA Improved mRNA translation of some gene reporters |
| 5-Iodo-U | 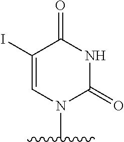 [SMILES: *n1cc(I)c(=O)[nH]c1=O] | Stabilize A-U pairs, tolerated in siRNA Improved mRNA translation of some gene reporters |
| N1-GalNAc-pseudoU | 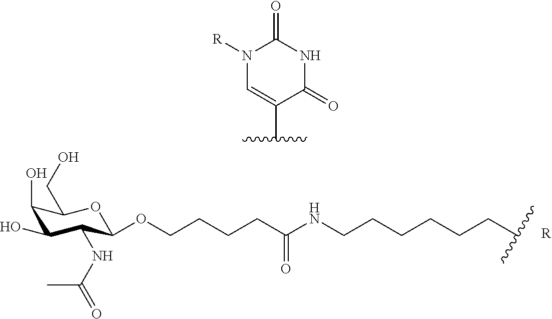 | Targeted delivery to hepatocytes |

In some embodiments, the nucleic acid comprises one or more chemically modified nucleotides of Table 4, one or more chemical backbone modifications of Table 5, one or more chemically modified caps of Table 5. For instance, in some embodiments, the nucleic acid comprises two or more (e.g., 3, 4, 5, 6, 7, 8, 9, or 10 or more) different types of chemical modifications. As an example, the nucleic acid may comprise two or more (e.g., 3, 4, 5, 6, 7, 8, 9, or 10 or more) different types of modified nucleobases, e.g., as described herein, e.g., in Table 4. Alternatively or in combination, the nucleic acid may comprise two or more (e.g., 3, 4, 5, 6, 7, 8, 9, or 10 or more) different types of backbone modifications, e.g., as described herein, e.g., in Table 5. Alternatively or in combination, the nucleic acid may comprise one or more modified cap, e.g., as described herein, e.g., in Table 6. For instance, in some embodiments, the nucleic acid comprises one or more type of modified nucleobase and one or more type of backbone modification; one or more type of modified nucleobase and one or more modified cap; one or more type of modified cap and one or more type of backbone modification; or one or more type of modified nucleobase, one or more type of backbone modification, and one or more type of modified cap.

In some embodiments, the nucleic acid comprises one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) modified nucleobases. In some embodiments, all nucleobases of the nucleic acid are modified. In some embodiments, the nucleic acid is modified at one or more (e.g., 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, or more) positions in the backbone. In some embodiments, all backbone positions of the nucleic acid are modified.

TABLE 4
Modified nucleotides

| | |
|---|---|
| 5-aza-uridine | N2-methyl-6-thio-guanosine |
| 2-thio-5-aza-midine | N2,N2-dimethyl-6-thio-guanosine |
| 2-thiouridine | pyridin-4-one ribonucleoside |
| 4-thio-pseudouridine | 2-thio-5-aza-uridine |
| 2-thio-pseudouridine | 2-thiomidine |
| 5-hydroxyuridine | 4-thio-pseudomidine |
| 3-methyluridine | 2-thio-pseudowidine |
| 5-carboxymethyl-uridine | 3-methylmidine |
| 1-carboxymethyl-pseudouridine | 1-propynyl-pseudomidine |
| 5-propynyl-uridine | 1-methyl-1-deaza-pseudomidine |
| 1-propynyl-pseudouridine | 2-thio-1-methyl-1-deaza-pseudouridine |
| 5-taurinomethyluridine | 4-methoxy-pseudomidine |
| 1-taurinomethyl-pseudouridine | 5′-O-(1-Thiophosphate)-Adenosine |
| 5-taurinomethyl-2-thio-uridine | 5′-O-(1-Thiophosphate)-Cytidine |
| 1-taurinomethyl-4-thio-uridine | 5′-O-(1-thiophosphate)-Guanosine |
| 5-methyl-uridine | 5′-O-(1-Thiophophate)-Uridine |
| 1-methyl-pseudouridine | 5′-O-(1-Thiophosphate)-Pseudouridine |
| 4-thio-1-methyl-pseudouridine | 2′-O-methyl-Adenosine |
| 2-thio-1-methyl-pseudouridine | 2′-O-methyl-Cytidine |
| 1-methyl-1-deaza-pseudouridine | 2′-O-methyl-Guanosine |
| 2-thio-1-methyl-1-deaza-pseudomidine | 2′-O-methyl-Uridine |
| dihydrouridine | 2′-O-methyl-Pseudouridine |
| dihydropseudouridine | 2′-O-methyl-Inosine |
| 2-thio-dihydromidine | 2-methyladenosine |
| 2-thio-dihydropseudouridine | 2-methylthio-N6-methyladenosine |
| 2-methoxyuridine | 2-methylthio-N6 isopentenyladenosine |
| 2-methoxy-4-thio-uridine | 2-methylthio-N6-(cis-hydroxyisopentenyl)adenosine |
| 4-methoxy-pseudouridine | N6-hydroxynorvalylcarbamoyladenosine |
| 4-methoxy-2-thio-pseudouridine | N6-methyl-N6-threonylcarbamoyladenosine |
| 5-aza-cytidine | 2-methylthio-N6-hydroxynorvalylcarbamoyladenosine |
| pseudoisocytidine | 2′-O-ribosyladenosine (phosphate) |
| 3-methyl-cytidine | 1,2′-O-dimethylinosine |
| N4-acetylcytidine | 5,2′-O-dimethylcytidine |
| 5-formylcytidine | N4-acetyl-2′-O-methylcytidine |
| N4-methylcytidine | Lysidine |
| 5-hydroxymethylcytidine | 7-methylguanosine |
| 1-methyl-pseudoisocytidine | N2,2′-O-dimethylguanosine |
| pyrrolo-cytidine | N2,N2,2′-O-trimethylguanosine |
| pyrrolo-pseudoisocytidine | 2′-O-ribosylguanosine (phosphate) |
| 2-thio-cytidine | Wybutosine |
| 2-thio-5-methyl-cytidine | Peroxywybutosine |
| 4-thio-pseudoisocytidine | Hydroxywybutosine |
| 4-thio-1-methyl-pseudoisocytidine | undermodified hydroxywybutosine |
| 4-thio-1-methyl-1-deaza-pseudoisocytidine | methylwyosine |
| 1-methyl-1-deaza-pseudoisocytidine | queuosine |
| zebularine | epoxyqueuosine |
| 5-aza-zebularine | galactosyl-queuosine |
| 5-methyl-zebularine | mannosyl-queuosine |
| 5-aza-2-thio-zebularine | 7-cyano-7-deazaguanosine |
| 2-thio-zebularine | 7-aminomethyl-7-deazaguanosine |
| 2-methoxy-cytidine | archaeosine |
| 2-methoxy-5-methyl-cytidine | 5,2′-O-dimethyluridine |
| 4-methoxy-pseudoisocytidine | 4-thiouridine |
| 4-methoxy-1-methyl-pseudoisocytidine | 5-methyl-2-thiouridine |
| 2-aminopurine | 2-thio-2′-O-methyluridine |
| 2,6-diaminopurine | 3-(3-amino-3-carboxypropyl)uridine |
| 7-deaza-adenine | 5-methoxyuridine |
| 7-deaza-8-aza-adenine | uridine 5-oxyacetic acid |
| 7-deaza-2-aminopurine | uridine 5-oxyacetic acid methyl ester |
| 7-deaza-8-aza-2-aminopurine | 5-(carboxyhydroxymethyl)uridine) |
| 7-deaza-2,6-diaminopurine | 5-(carboxyhydroxymethyl)uridine methyl ester |
| 7-deaza-8-aza-2,6-diarninopurine | 5-methoxycarbonylmethyluridine |
| 1-methyladenosine | 5-methoxycarbonylmethyl-2′-O-methyluridine |
| N6-isopentenyladenosine | 5-methoxycarbonylmethyl-2-thiouridine |
| N6-(cis-hydroxyisopentenyl)adenosine | 5-aminomethyl-2-thiouridine |
| 2-methylthio-N6-(cis-hydroxyisopentenyl)adenosine | 5-methylaminomethyluridine |
| N6-glycinylcarbamoyladenosine | 5-methylaminomethyl-2-thiouridine |
| N6-threonylcarbamoyladenosine | 5-methylaminomethyl-2-selenouridine |
| 2-methylthio-N6-threonylcarbamoyladenosine | 5-carbamoylmethyluridine |
| N6,N6-dimethyladenosine | 5-carbamoylmethyl-2′-O-methyluridine |
| 7-methyladenine | 5-carboxymethylaminomethyluridine |
| 2-methylthio-adenine | 5-carboxymethylaminomethyl-2′-O-methyluridine |
| 2-methoxy-adenine | 5-carboxymethylaminomethyl-2-thiouridine |
| inosine | N4,2′-O-dimethylcytidine |
| 1-methyl-inosine | 5-carboxymethyluridine |
| wyosine | N6,2′-O-dimethyladenosine |
| wybutosine | N,N6,O-2′-trimethyladenosine |
| 7-deaza-guanosine | N2,7-dimethylguanosine |
| 7-deaza-8-aza-guanosine | N2,N2,7-trimethylguanosine |
| 6-thio-guanosine | 3,2′-O-dimethyluridine |
| 6-thio-7-deaza-guanosine | 5-methyldihydrouridine |
| 6-thio-7-deaza-8-aza-guanosine | 5-formyl-2′-O-methylcytidine |
| 7-methyl-guanosine | 1,2′-O-dimethylguanosine |
| 6-thio-7-methyl-guanosine | 4-demethylwyosine |
| 7-methylinosine | Isowyosine |
| 6-methoxy-guanosine | N6-acetyladenosine |
| 1-methylguanosine | |
| N2-methylguanosine | |
| N2,N2-dimethylguanosine | |
| 8-oxo-guanosine | |
| 7-methyl-8-oxo-guanosine | |
| 1-methyl-6-thio-guanosine | |

TABLE 5
Backbone modifications

2′-O-Methyl backbone
Peptide Nucleic Acid (PNA) backbone
phosphorothioate backbone
morpholino backbone
carbamate backbone
siloxane backbone
sulfide backbone
sulfoxide backbone
sulfone backbone
formacetyl backbone
thioformacetyl backbone
methyleneformacetyl backbone
riboacetyl backbone
alkene containing backbone
sulfamate backbone
sulfonate backbone
sulfonamide backbone
methyleneimino backbone
methylenehydrazino backbone
amide backbone

TABLE 6
Modified caps

m7GpppA
m7GpppC
m2,7GpppG
m2,2,7GpppG
m7Gpppm7G
m7,2′OmeGpppG
m72′dGpppG
m7,3′OmeGpppG
m7,3′dGpppG
GppppG
m7GppppG
m7GppppA
m7GppppC
m2,7GppppG
m2,2,7GppppG
m7Gppppm7G
m7,2′OmeGppppG
m72′dGppppG
m7,3′OmeGppppG
m7,3′dGppppG

Compositions for RNA and Modified RNA (e.g., gRNA or Template RNA)

In some embodiments, the template nucleic acid is a template RNA. In some embodiments, the template RNA comprises one or more modified nucleotides. For example, in some embodiments, the template RNA comprises one or more deoxyribonucleotides. In some embodiments, regions of the template RNA are replaced by DNA nucleotides, e.g., to enhance stability of the molecule. For example, the 3′ end of the template may comprise DNA nucleotides, while the rest of the template comprises RNA nucleotides that can be reverse transcribed. For instance, in some embodiments, the heterologous object sequence is primarily or wholly made up of RNA nucleotides (e.g., at least 90%, 95%, 98%, or 99% RNA nucleotides). In some embodiments, one or both of the 3′ UTR and the 3′ target homology domain are primarily or wholly made up of DNA nucleotides (e.g., at least 90%, 95%, 98%, or 99% DNA nucleotides). In other embodiments, the template region for writing into the genome may comprise DNA nucleotides. In some embodiments, the DNA nucleotides in the template are copied into the genome by a domain capable of DNA-dependent DNA polymerase activity. In some embodiments, the DNA-dependent DNA polymerase activity is provided by a DNA polymerase domain in the polypeptide. In some embodiments, the DNA-dependent DNA polymerase activity is provided by a reverse transcriptase domain that is also capable of DNA-dependent DNA polymerization, e.g., second strand synthesis. In some embodiments, the template molecule is composed of only DNA nucleotides.

The nucleotides comprising the template of the system can be natural or modified bases, or a combination thereof. For example, the template may contain pseudouridine, dihydrouridine, inosine, 7-methylguanosine, or other modified bases. In some embodiments, the template may contain locked nucleic acid nucleotides. In some embodiments, the modified bases used in the template do not inhibit the reverse transcription of the template. In some embodiments, the modified bases used in the template may improve reverse transcription, e.g., specificity or fidelity.

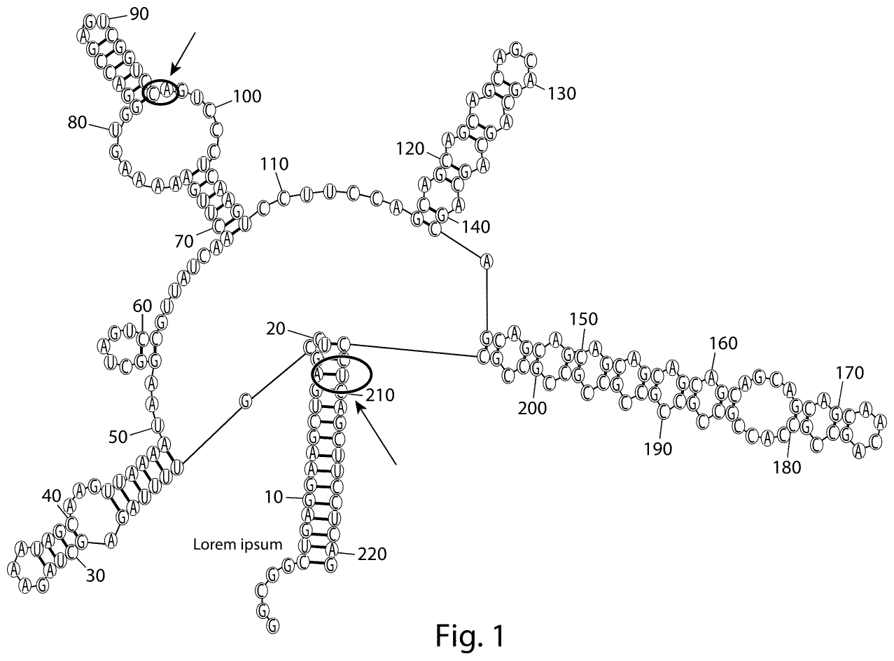

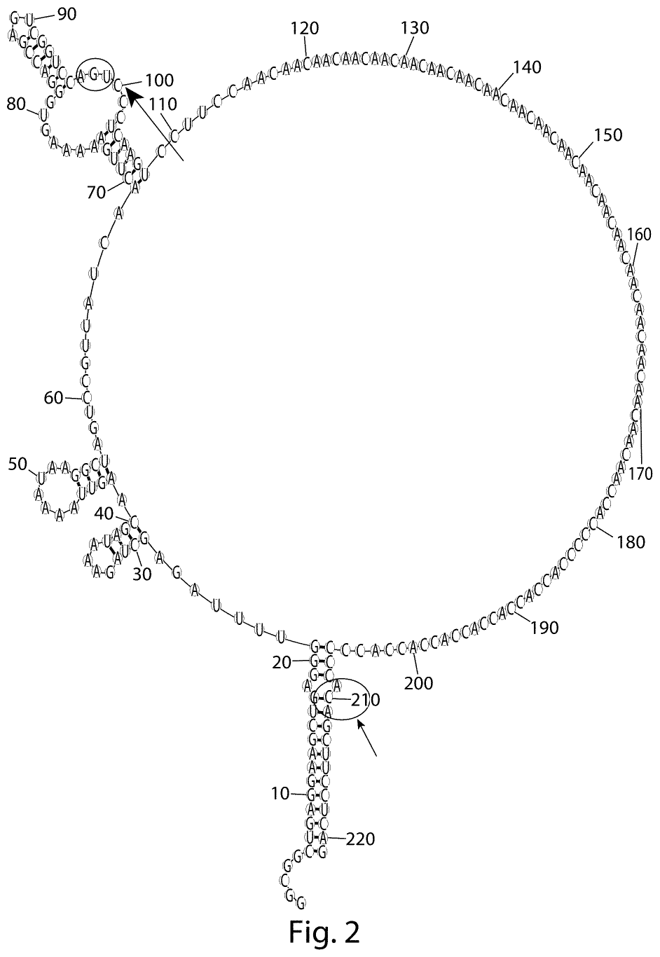

In some embodiments, an RNA component of the system (e.g., a template RNA or a gRNA) comprises one or more nucleotide modifications. In some embodiments, the modification pattern of a gRNA can significantly affect in vivo activity compared to unmodified or end-modified guides (e.g., as shown in FIG. 1D from Finn et al. Cell Rep 22(9):2227-2235 (2018); incorporated herein by reference in its entirety). Without wishing to be bound by theory, this process may be due, at least in part, to a stabilization of the RNA conferred by the modifications. Non-limiting examples of such modifications may include 2′-O-methyl (T-O-Me), 2′-O-(2-methoxyethyl) (2′-O-MOE), 2′-fluoro (2′-F), phosphorothioate (PS) bond between nucleotides, G-C substitutions, and inverted abasic linkages between nucleotides and equivalents thereof.

In some embodiments, the template RNA (e.g., at the portion thereof that binds a target site) or the guide RNA comprises a 5′ terminus region. In some embodiments, the template RNA or the guide RNA does not comprise a 5′ terminus region. In some embodiments, the 5′ terminus region comprises a CRISPR spacer region, e.g., as described with respect to sgRNA in Briner A E et al, Molecular Cell 56: 333-339 (2014) (incorporated herein by reference in its entirety; applicable herein, e.g., to all guide RNAs). In some embodiments, the 5′ terminus region comprises a 5′ end modification. In some embodiments, a 5′ terminus region with or without a spacer region may be associated with a crRNA, trRNA, sgRNA and/or dgRNA. The CRISPR spacer region can, in some instances, comprise a guide region, guide domain, or targeting domain. In some embodiments, a target domain or target sequence may comprise a sequence of nucleic acid to which the guide region/domain directs a nuclease for cleavage. In some embodiments, a spyCas9 protein may be directed by a guide region/domain to a target sequence of a target nucleic acid molecule by the nucleotides present in the CRISPR spacer region.

In some embodiments, the template RNAs (e.g., at the portion thereof that binds a target site) or the gRNA comprises a 2′-O-methyl (2′-O-Me) modified nucleotide. In some embodiments, the gRNA comprises a 2′-O-(2-methoxy ethyl) (2′-O-moe) modified nucleotide. In some embodiments, the gRNA comprises a 2′-fluoro (2′-F) modified nucleotide. In some embodiments, the gRNA comprises a phosphorothioate (PS) bond between nucleotides. In some embodiments, the gRNA comprises a 5′ end modification, a 3′ end modification, or 5′ and 3′ end modifications. In some embodiments, the 5′ end modification comprises a phosphorothioate (PS) bond between nucleotides. In some embodiments, the 5′ end modification comprises a 2′-O-methyl (2′-O-Me), 2′-O-(2-methoxy ethyl) (2′-O-MOE), and/or 2′-fluoro (2′-F) modified nucleotide. In some embodiments, the 5′ end modification comprises at least one phosphorothioate (PS) bond and one or more of a 2′-O-methyl (2′-O-Me), 2′-O-(2-methoxyethyl) (2′-O-MOE), and/or 2′-fluoro (2′-F) modified nucleotide. The end modification may comprise a phosphorothioate (PS), 2′-O-methyl (2′-O-Me), 2′-O-(2-methoxyethyl) (2′-O-MOE), and/or 2′-fluoro (2′-F) modification. Equivalent end modifications are also encompassed by embodiments described herein. In some embodiments, the template RNA or gRNA comprises an end modification in combination with a modification of one or more regions of the template RNA or gRNA. Additional exemplary modifications and methods for protecting RNA, e.g., gRNA, and formulae thereof, are described in WO2018126176A1, which is incorporated herein by reference in its entirety.

In some embodiments, structure-guided and systematic approaches are used to introduce modifications (e.g., 2′-OMe-RNA, 2′-F-RNA, and PS modifications) to a template RNA or guide RNA, for example, as described in Mir et al. Nat Commun 9:2641 (2018) (incorporated by reference herein in its entirety). In some embodiments, the incorporation of 2′-F-RNAs increases thermal and nuclease stability of RNA:RNA or RNA:DNA duplexes, e.g., while minimally interfering with C3′-endo sugar puckering. In some embodiments, 2′-F may be better tolerated than 2′-OMe at positions where the 2′-OH is important for RNA:DNA duplex stability. In some embodiments, a crRNA comprises one or more modifications that do not reduce Cas9 activity, e.g., C10, C20, or C21 (fully modified), e.g., as dscribed in Supplementary Table 1 of Mir et al. Nat Commun 9:2641 (2018), incorporated herein by reference in its entirety. In some embodiments, a tracrRNA comprises one or more modifications that do not reduce Cas9 activity, e.g., T2, T6, T7, or T8 (fully modified) of Supplementary Table 1 of Mir et al. Nat Commun 9:2641 (2018). In some embodiments, a crRNA comprises one or more modifications (e.g., as described herein) may be paired with a tracrRNA comprising one or more modifications, e.g., C20 and T2. In some embodiments, a gRNA comprises a chimera, e.g., of a crRNA and a tracrRNA (e.g., Jinek et al. Science 337(6096):816-821 (2012)). In embodiments, modifications from the crRNA and tracrRNA are mapped onto the single-guide chimera, e.g., to produce a modified gRNA with enhanced stability.

In some embodiments, structure-guided and systematic approaches (e.g., as described in Mir et al. Nat Commun 9:2641 (2018); incorporated herein by reference in its entirety) are employed to find optimal modifications for the template RNA. In embodiments, the modifications are identified with the inclusion or exclusion of a guide region of the template RNA. In some embodiments, a structure of polypeptide bound to template RNA is used to determine non-protein-contacted nucleotides of the RNA that may then be selected for modifications, e.g., with lower risk of disrupting the association of the RNA with the polypeptide.

In some embodiments, a template nucleic acid (e.g., template RNA) comprises a heterologous object sequence comprising a sequence given in Table 7.

### Additional Nucleic Acid Components of Gene Editor Systems

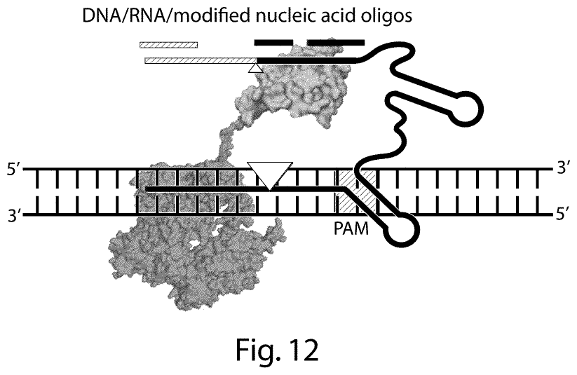

As mentioned above, in some embodiments it is desirable to reduce secondary structure in a template RNA described herein. This disclosure provides several strategies for reducing secondary structure. One such strategy is illustrated in FIG. 12. FIG. 12 depicts a template RNA and corresponding protein, with oligonucleotides that pair with specific regions of the template RNA and disrupt pairing within the template RNA.

Accordingly, in some embodiments, a system described herein further comprises an oligonucleotide with complementarity (e.g., perfect complementarity) to the template RNA. In some embodiments, the oligonucleotide is complementary to one or both of (iii) the heterologous object sequence, and (iv) the 3′ target homology domain. In some embodiments, the oligonucleotide is complementary to a region of (iv), wherein the region of (iv) has complementarity with a second region in the template RNA, e.g., wherein the second region is a region in (iii) or (i). In some embodiments, the oligonucleotide is complementary to a region of (iii), wherein the region of (iii) has complementarity with a second region in the template RNA, e.g., wherein the second region is a region in (iii) or (i).

In some embodiments, the oligonucleotide has a sufficiently weak affinity for the template RNA that the oligonucleotide does not interfere with reverse transcriptase activity and integration of new sequence into the target DNA.

In some embodiments, the oligonucleotide comprises DNA or RNA. In some embodiments, the oligonucleotide comprises one or more chemical modifications, e.g., as described herein. In some embodiments, the chemical modification comprises 2′-OMe, 2′F, or LNA. In some embodiments, the oligonucleotide has a length of about 5, 6, 7, 8, 9, or 10, nucleotides.

In some embodiments, the system comprises one, two, or three different oligonucleotides.

In some embodiments, a method herein comprises providing a template RNA and an oligonucleotide described herein. In some embodiments, the method comprises incubating the template RNA and the oligonucleotide under conditions that allow for binding.

### Polypeptide Components of Gene Editor System

Domains and Functions:

In some embodiments, a polypeptide as described herein (e.g., comprising an RT domain, e.g., a genome editing polypeptide as described herein) possesses the functions of DNA target site binding, template nucleic acid (e.g., RNA) binding, DNA target site cleavage, and template nucleic acid (e.g., RNA) writing, e.g., reverse transcription. In some embodiments, each functions is contained within a distinct domain. In some embodiments, a function may be attributed to two or more domains (e.g., two or more domains, together, exhibit the functionality). In some embodiments, two or more domains may have the same or similar function (e.g., two or more domains each independently have DNA-binding functionality, e.g., for two different DNA sequences). In other embodiments, one or more domains may be capable of enabling one or more functions, e.g., a Cas9 domain enabling both DNA binding and target site cleavage. In some embodiments, the domains are all located within a single polypeptide. In some embodiments, a first domain is in one polypeptide and a second domain is in a second polypeptide. For example, in some embodiments, the polypeptide may be split between a first polypeptide and a second polypeptide, e.g., wherein the first polypeptide comprises a reverse transcriptase (RT) domain and wherein the second polypeptide comprises a DNA-binding domain and an endonuclease domain, e.g., a nickase domain. As a further example, in some embodiments, the first polypeptide and the second polypeptide each comprise a DNA binding domain (e.g., a first DNA binding domain and a second DNA binding domain). In some embodiments, the first and second polypeptide may be brought together post-translationally via a split-intein.

Writing Domain:

In certain aspects of the present invention, the writing domain of the system possesses reverse transcriptase activity and is also referred to as a reverse transcriptase domain (a RT domain). In some embodiments, the RT domain comprises an RT catalytic portion and RNA-binding region (e.g., a region that binds the template RNA).

In certain aspects of the present invention, the writing domain is based on a reverse transcriptase domain of an APE-type or RLE-type non-LTR retrotransposon. A wild-type reverse transcriptase domain of an APE-type or RLE-type non-LTR retrotransposon can be used in a system or can be modified (e.g., by insertion, deletion, or substitution of one or more residues) to alter the reverse transcriptase activity for target DNA sequences. In some embodiments the reverse transcriptase is altered from its natural sequence to have altered codon usage, e.g. improved for human cells. In some embodiments the reverse transcriptase domain is a heterologous reverse transcriptase from a different retrovirus, LTR-retrotransposon, or non-LTR retrotransposon.

Template Nucleic Acid Binding Domain:

The polypeptide typically contains regions capable of associating with the template nucleic acid (e.g., template RNA). In some embodiments, the template nucleic acid binding domain is an RNA binding domain. In some embodiments, the RNA binding domain is a modular domain that can associate with RNA molecules containing specific signatures, e.g., structural motifs, e.g., secondary structures present in the 3′ UTR in non-LTR retrotransposons. In other embodiments, the template nucleic acid binding domain (e.g., RNA binding domain) is contained within the reverse transcription domain, e.g., the reverse transcriptase-derived component has a known signature for RNA preference, e.g., secondary structures present in the 3′ UTR in non-LTR retrotransposons. In other embodiments, the template nucleic acid binding domain (e.g., RNA binding domain) is contained within the DNA binding domain. For example, in some embodiments, the DNA binding domain is a CRISPR-associated protein that recognizes the structure of a template nucleic acid (e.g., template RNA) comprising a gRNA. In some embodiments, the gRNA is a short synthetic RNA composed of a scaffold sequence that participates in CRISPR-associated protein binding and a user-defined ˜20 nucleotide targeting sequence for a genomic target.

Endonuclease Domain:

In some embodiments, a polypeptide possesses the function of DNA target site cleavage via an endonuclease domain. In some embodiments, the endonuclease domain is also a DNA-binding domain. In some embodiments, the endonuclease domain is also a template nucleic acid (e.g., template RNA) binding domain. For example, in some embodiments a polypeptide comprises a CRISPR-associated endonuclease domain that binds a template RNA comprising a gRNA, binds a target DNA sequence (e.g., with complementarity to a portion of the gRNA), and cuts the target DNA sequence. In certain embodiments, the endonuclease/DNA binding domain of an APE-type retrotransposon or the endonuclease domain of an RLE-type retrotransposon can be used or can be modified (e.g., by insertion, deletion, or substitution of one or more residues) in a system described herein. In some embodiments the endonuclease domain or endonuclease/DNA binding domain is altered from its natural sequence to have altered codon usage, e.g. improved for human cells. In some embodiments the endonuclease element is a heterologous endonuclease element, such as Fok1 nuclease, a type-II restriction 1-like endonuclease (RLE-type nuclease), or another RLE-type endonuclease (also known as REL). In some embodiments the heterologous endonuclease activity has nickase activity and does not form double stranded breaks.

DNA Binding Domain:

In certain aspects, the DNA-binding domain of a polypeptide described herein is selected, designed, or constructed for binding to a desired host DNA target sequence. In certain embodiments, the DNA-binding domain of the polypeptide is a heterologous DNA-binding protein or domain relative to a native retrotransposon sequence. In some embodiments the heterologous DNA binding element is a zinc-finger element or a TAL effector element, e.g., a zinc-finger or TAL polypeptide or functional fragment thereof. In some embodiments the heterologous DNA binding element is a sequence-guided DNA binding element, such as Cas9, Cpf1, or other CRISPR-related protein that has been altered to have no endonuclease activity. In some embodiments the heterologous DNA binding element retains endonuclease activity. In some embodiments, the heterologous DNA binding element retains partial endonuclease activity to cleave ssDNA, e.g., possesses nickase activity.

Linkers

In some embodiments, domains of the compositions and systems described herein (e.g., the endonuclease and reverse transcriptase domains of a polypeptide or the DNA binding domain and reverse transcriptase domains of a polypeptide) may be joined by a linker. A composition described herein comprising a linker element has the general form S1-L-S2, wherein S1 and S2 may be the same or different and represent two domain moieties (e.g., each a polypeptide or nucleic acid domain) associated with one another by the linker. In some embodiments, a linker may connect two polypeptides. In some embodiments, a linker may connect two nucleic acid molecules. In some embodiments, a linker may connect a polypeptide and a nucleic acid molecule. A linker may be a chemical bond, e.g., one or more covalent bonds or non-covalent bonds. A linker may be flexible, rigid, and/or cleavable. In some embodiments, the linker is a peptide linker. Generally, a peptide linker is at least 2, 3, 4, 5, 6, 7, 8, 9, 10 or more amino acids in length, e.g., 2-50 amino acids in length, 2-30 amino acids in length.

### Resolution of Editing Events

After writing of the template nucleic acid into the target site, additional activities may be performed to increase the overall efficiency of incorporation. In some embodiments, a nick may be initiated in the genome on the non-written DNA strand to encourage copying of the newly written DNA onto the second strand. In some embodiments, the nick may be within at least 10, 20, 30, 40, 50, 60, 70 80, 90, or 100 bases of the target site. In some embodiments, this second nick is performed by the same polypeptide performing the writing. In other embodiments, the second nick may be performed by an additional polypeptide encoding nickase activity, e.g. a Cas9 nickase.

For some systems, the writing process may leave a 3′ flap containing the newly written DNA that must displace the flanking target sequence to anneal to the second genomic strand to complete the edit. In some embodiments, the 3′ flap is designed to have enhanced strand invasion capability. In some embodiments, 5′-3′ exonuclease activity is supplemented to chew back the exposed 5′ end of the displaced strand. In some embodiments, DNA ligase activity is supplemented to complete the reaction. In some embodiments, the exonuclease and/or ligase activities are optionally provided on the polypeptide. In some embodiments, the exonuclease and/or ligase activities are optionally provided separately from the polypeptide.

Based on the published mechanism of non-LTR retrotransposons, systems derived therefrom may not require supplementation of additional functions for resolution of the writing event. In some embodiments, the system may result in complete writing without requiring endogenous host factors. In some embodiments, the system may result in complete writing without the need for DNA repair. In some embodiments, the system may result in complete writing without eliciting a DNA damage response.

### Production of Compositions and Systems

As will be appreciated by one of skill, methods of designing and constructing nucleic acid constructs and proteins or polypeptides (such as the systems, constructs and polypeptides described herein) are routine in the art. Generally, recombinant methods may be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012). The disclosure provides, in part, a nucleic acid, e.g., vector, encoding a polypeptide described herein (e.g., comprising an RT domain, e.g., a genome editing polypeptide as described herein), a template nucleic acid described herein, or both.

In some embodiments, a system, polypeptide, and/or template nucleic acid (e.g., template RNA), e.g., as described herein, conforms to certain quality standards. In some embodiments, a system, polypeptide, and/or template nucleic acid (e.g., template RNA), e.g., as described herein, produced by a method described herein conforms to certain quality standards. Accordingly, the disclosure is directed in part to methods of manufacturing a system, polypeptide, and/or template nucleic acid (e.g., template RNA), e.g., as described herein, that conforms to certain quality standards, e.g., in which said quality standards are assayed. The disclosure is further directed to methods of assaying said quality standards in a system, polypeptide, and/or template nucleic acid (e.g., template RNA), e.g., as described herein. In some embodiments, quality standards include, but are not limited to:
(i) the length of the template RNA, e.g., whether the template RNA has a length that is above a reference length or within a reference length range, e.g., whether at least 80, 85, 90, 95, 96, 97, 98, or 99% of the template RNA present is greater than 100, 125, 150, 175, or 200 nucleotides long;
(ii) the presence, absence, and/or length of a polyA tail on the template RNA, e.g., whether at least 80, 85, 90, 95, 96, 97, 98, or 99% of the template RNA present contains a polyA tail (e.g., a polyA tail that is at least 5, 10, 20, 30, 50, 70, 100 nucleotides in length);
(iii) the presence, absence, and/or type of a 5′ cap on the template RNA, e.g., whether at least 80, 85, 90, 95, 96, 97, 98, or 99% of the template RNA present contains a 5′ cap, e.g., whether that cap is a 7-methylguanosine cap, e.g., a O-Me-m7G cap;
(iv) the presence, absence, and/or type of one or more modified nucleotides (e.g., selected from pseudouridine, dihydrouridine, inosine, 7-methylguanosine, 1-N-methylpseudouridine (1-Me-Ψ), 5-methoxyuridine (5-MO-U), 5-methylcytidine (5mC), or a locked nucleotide) in the template RNA, e.g., whether at least 80, 85, 90, 95, 96, 97, 98, or 99% of the template RNA present contains one or more modified nucleotides;
(v) the stability of the template RNA (e.g., over time and/or under a pre-selected condition), e.g., whether at least 80, 85, 90, 95, 96, 97, 98, or 99% of the template RNA remains intact (e.g., greater than 100, 125, 150, 175, or 200 nucleotides long) after a stability test; (vi) the potency of the template RNA in a system for modifying DNA, e.g., whether at least 1% of target sites are modified after a system comprising the template RNA is assayed for potency;
(vii) the length of the polypeptide, first polypeptide, or second polypeptide, e.g., whether the polypeptide, first polypeptide, or second polypeptide has a length that is above a reference length or within a reference length range, e.g., whether at least 80, 85, 90, 95, 96, 97, 98, or 99% of the polypeptide, first polypeptide, or second polypeptide present is greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long);
(viii) the presence, absence, and/or type of post-translational modification on the polypeptide, first polypeptide, or second polypeptide, e.g., whether at least 80, 85, 90, 95, 96, 97, 98, or 99% of the polypeptide, first polypeptide, or second polypeptide contains phosphorylation, methylation, acetylation, myristoylation, palmitoylation, isoprenylation, glypiation, or lipoylation, or any combination thereof;
(ix) the presence, absence, and/or type of one or more artificial, synthetic, or non-canonical amino acids (e.g., selected from ornithine, β-alanine, GABA, δ-Aminolevulinic acid, PABA, a D-amino acid (e.g., D-alanine or D-glutamate), aminoisobutyric acid, dehydroalanine, cystathionine, lanthionine, Djenkolic acid, Diaminopimelic acid, Homoalanine, Norvaline, Norleucine, Homonorleucine, homoserine, O-methyl-homoserine and O-ethyl-homoserine, ethionine, selenocysteine, selenohomocysteine, selenomethionine, selenoethionine, tellurocysteine, or telluromethionine) in the polypeptide, first polypeptide, or second polypeptide, e.g., whether at least 80, 85, 90, 95, 96, 97, 98, or 99% of the polypeptide, first polypeptide, or second polypeptide present contains one or more artificial, synthetic, or non-canonical amino acids;
(x) the stability of the polypeptide, first polypeptide, or second polypeptide (e.g., over time and/or under a pre-selected condition), e.g., whether at least 80, 85, 90, 95, 96, 97, 98, or 99% of the polypeptide, first polypeptide, or second polypeptide remains intact (e.g., greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long)) after a stability test;
(xi) the potency of the polypeptide, first polypeptide, or second polypeptide in a system for modifying DNA, e.g., whether at least 1% of target sites are modified after a system comprising the polypeptide, first polypeptide, or second polypeptide is assayed for potency; or
(xii) the presence, absence, and/or level of one or more of a pyrogen, virus, fungus, bacterial pathogen, or host cell protein, e.g., whether the system is free or substantially free of pyrogen, virus, fungus, bacterial pathogen, or host cell protein contamination.

Further included here are compositions and methods for the assembly of full or partial template RNA molecules (e.g., template RNA molecules optionally comprising a gRNA, or separate gRNA molecules). In some embodiments, RNA molecules may be assembled by the connection of two or more (e.g., two, three, four, five, six, seven, eight, nine, ten, or more) RNA segments with each other. In an aspect, the disclosure provides methods for producing nucleic acid molecules, the methods comprising contacting two or more linear RNA segments with each other under conditions that allow for the 5′ terminus of a first RNA segment to be covalently linked with the 3′ terminus of a second RNA segment. In some embodiments, the joined molecule may be contacted with a third RNA segment under conditions that allow for the 5′ terminus of the joined molecule to be covalently linked with the 3′ terminus of the third RNA segment. In embodiments, the method further comprises joining a fourth, fifth, or additional RNA segments to the elongated molecule. This form of assembly may, in some instances, allow for rapid and efficient assembly of RNA molecules.

The present disclosure also provides compositions and methods for the connection (e.g., covalent connection) of crRNA molecules and tracrRNA molecules. In some embodiments, guide RNA molecules with specificity for different target sites can be generated using a single tracrRNA molecule/segment connected to a target site specific crRNA molecule/segment (e.g., as shown in FIG. 10 of US20160102322A1; incorporated herein by reference in its entirety). For example, FIG. 10 of US20160102322A1 shows four tubes with different crRNA molecules with crRNA molecule 3 being connected to a tracrRNA molecule to form a guide RNA molecule, thereby depicting an exemplary connection of two RNA segments to form a product RNA molecule.

The disclosure also provides compositions and methods for the production of template RNA molecules with specificity for a polypeptide as described herein (e.g., comprising an RT domain, e.g., a genome editing polypeptide as described herein) and/or a genomic target site. In an aspect, the method comprises: (1) identification of the target site and desired modification thereto, (2) production of RNA segments including an upstream homology segment, a heterologous object sequence segment, a polypeptide binding motif, and a gRNA segment, and/or (3) connection of the four or more segments into at least one molecule, e.g., into a single RNA molecule. In some embodiments, some or all of the template RNA segments comprised in (2) are assembled into a template RNA molecule, e.g., one, two, three, or four of the listed components. In some embodiments, the segments comprised in (2) may be produced in further segmented molecules, e.g., split into at least 2, at least 3, at least 4, or at least 5 or more sub-segments, e.g., that are subsequently assembled, e.g., by one or more methods described herein.

In some embodiments, RNA segments may be produced by chemical synthesis. In some embodiments, RNA segments may be produced by in vitro transcription of a nucleic acid template, e.g., by providing an RNA polymerase to act on a cognate promoter of a DNA template to produce an RNA transcript. In some embodiments, in vitro transcription is performed using, e.g., a T7, T3, or SP6 RNA polymerase, or a derivative thereof, acting on a DNA, e.g., dsDNA, ssDNA, linear DNA, plasmid DNA, linear DNA amplicon, linearized plasmid DNA, e.g., encoding the RNA segment, e.g., under transcriptional control of a cognate promoter, e.g., a T7, T3, or SP6 promoter. In some embodiments, a combination of chemical synthesis and in vitro transcription is used to generate the RNA segments for assembly. In embodiments, the gRNA, upstream target homology, and polypeptide binding segments are produced by chemical synthesis and the heterologous object sequence segment is produced by in vitro transcription. Without wishing to be bound by theory, in vitro transcription may be better suited for the production of longer RNA molecules. In some embodiments, reaction temperature for in vitro transcription may be lowered from optimal, e.g., be less than 37° C., to result in a higher proportion of full-length transcripts (Krieg Nucleic Acids Res 18:6463 (1990)). In some embodiments, a protocol for improved synthesis of long transcripts is employed to synthesize a long template RNA, e.g., a template RNA greater than 5 kb, such as the use of e.g., T7 RiboMAX Express, which can generate 27 kb transcripts in vitro (Thiel et al. J Gen Virol 82(6):1273-1281 (2001)). In some embodiments, modifications to RNA molecules as described herein may be incorporated during synthesis of RNA segments (e.g., through the inclusion of modified nucleotides or alternative binding chemistries), following synthesis of RNA segments through chemical or enzymatic processes, following assembly of one or more RNA segments, or a combination thereof.

In some embodiments, an mRNA of the system (e.g., an mRNA encoding a polypeptide as described herein, e.g., comprising an RT domain, e.g., a genome editing polypeptide as described herein) is synthesized in vitro using T7 polymerase-mediated DNA-dependent RNA transcription from a linearized DNA template, where UTP is optionally substituted with 1-methylpseudoUTP. In some embodiments, the transcript incorporates 5′ and 3′ UTRs, e.g., GGGAAAUAAGAGAGAAAAGAAGAGUAAGAAGAAAUAUAAGAGCCACC (SEQ ID NO: 56) and UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA (SEQ ID NO: 4), and optionally includes a poly-A tail, which can be encoded in the DNA template or added enzymatically following transcription. In some embodiments, a donor methyl group, e.g., S-adenosylmethionine, is added to a methylated capped RNA with cap 0 structure to yield a cap 1 structure that increases mRNA translation efficiency (Richner et al. Cell 168(6): P1114-1125 (2017)).

In some embodiments, the transcript from a T7 promoter starts with a GGG motif. In some embodiments, a transcript from a T7 promoter does not start with a GGG motif. It has been shown that a GGG motif at the transcriptional start, despite providing superior yield, may lead to T7 RNAP synthesizing a ladder of poly(G) products as a result of slippage of the transcript on the three C residues in the template strand from +1 to +3 (Imburgio et al. Biochemistry 39(34):10419-10430 (2000). For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

In some embodiments, RNA segments may be connected to each other by covalent coupling. In some embodiments, an RNA ligase, e.g., T4 RNA ligase, may be used to connect two or more RNA segments to each other. When a reagent such as an RNA ligase is used, a 5′ terminus is typically linked to a 3′ terminus. In some embodiments, if two segments are connected, then there are two possible linear constructs that can be formed (i.e., (1) 5′-Segment 1-Segment 2-3′ and (2) 5′-Segment 2-Segment 1-3′). In some embodiments, intramolecular circularization can also occur. Both of these issues can be addressed, for example, by blocking one 5′ terminus or one 3′ terminus so that RNA ligase cannot ligate the terminus to another terminus. In embodiments, if a construct of 5′-Segment 1-Segment 2-3′ is desired, then placing a blocking group on either the 5′ end of Segment 1 or the 3′ end of Segment 2 may result in the formation of only the correct linear ligation product and/or prevent intramolecular circularization. Compositions and methods for the covalent connection of two nucleic acid (e.g., RNA) segments are disclosed, for example, in US20160102322A1 (incorporated herein by reference in its entirety), along with methods including the use of an RNA ligase to directionally ligate two single-stranded RNA segments to each other.

One example of an end blocker that may be used in conjunction with, for example, T4 RNA ligase, is a dideoxy terminator. T4 RNA ligase typically catalyzes the ATP-dependent ligation of phosphodiester bonds between 5′-phosphate and 3′-hydroxyl termini. In some embodiments, when T4 RNA ligase is used, suitable termini must be present on the termini being ligated. One means for blocking T4 RNA ligase on a terminus comprises failing to have the correct terminus format. Generally, termini of RNA segments with a 5-hydroxyl or a 3′-phosphate will not act as substrates for T4 RNA ligase.

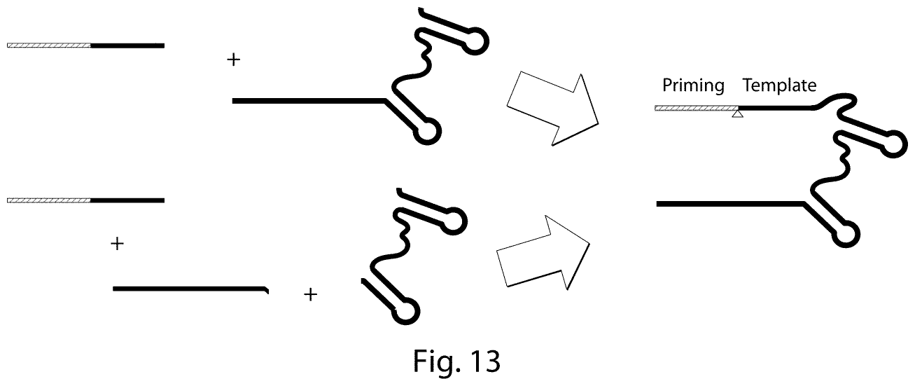

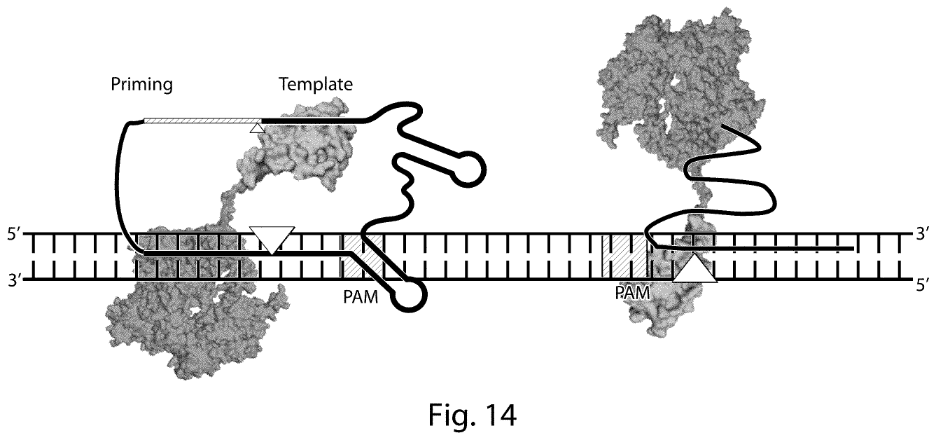

In some embodiments, a template RNA is produced as a plurality of segments (e.g., wherein each segment is made by solid phase synthesis) and the segments are then joined. Such a process is illustrated in FIG. 13. In some embodiment, the first segment comprises (iii) a heterologous object sequence (labeled “template” in FIG. 13), and (iv) a 3′ target homology domain (labeled “priming” in FIG. 13). In some embodiments, the second segment comprises (ii) a sequence that binds the RT domain and/or endonuclease domain. In some embodiments, the second segment further comprises (i) the sequence that binds a target site in the DNA. In some embodiments, (i) is present on a third segment. Various methods are provided herein for connecting the RNA segments.

For instance, RNA segments can be connected using splint ligation. Typically, in splint ligation, a splint oligonucleotide is provided that can pair with the first and second segments to be joined. Thus, in some embodiments, the splint oligonucleotide has a first region of complementarity to one end (e.g., the 5′ end) of the first RNA segment and a second region of complementarity to a compatible end (e.g., the 3′ end) of the second segment. The splint oligonucleotide therefore can bring the compatible ends of the first and second RNAs into proximity. A ligase (e.g., T4 DNA ligase) can then be added under conditions that allow for covalent linkage of the compatible ends of the first and second RNA segments. In some embodiments, the splint oligonucleotide comprises DNA.

Additional exemplary methods that may be used to connect RNA segments is by click chemistry (e.g., as described in U.S. Pat. Nos. 7,375,234 and 7,070,941, and US Patent Publication No. 2013/0046084, the entire disclosures of which are incorporated herein by reference). For example, one exemplary click chemistry reaction is between an alkyne group and an azide group (see FIG. 11 of US20160102322A1, which is incorporated herein by reference in its entirety). Any click reaction may potentially be used to link RNA segments (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). In some embodiments, ligation of RNA molecules using a click chemistry reaction is advantageous because click chemistry reactions are fast, modular, efficient, often do not produce toxic waste products, can be done with water as a solvent, and/or can be set up to be stereospecific. In some embodiments, the click chemistry used is a chemistry described in WO2016065364A1, which is incorporated herein by reference.

In some embodiments, the RNA segments are connected by a structure as shown in Table 8. In some embodiments, the RNA segments are connected by a structure as shown in Table 9.

In some embodiments, RNA segments may be connected using an Azide-Alkyne Huisgen Cycloaddition. reaction, which is typically a 1,3-dipolar cycloaddition between an azide and a terminal or internal alkyne to give a 1,2,3-triazole for the ligation of RNA segments. Without wishing to be bound by theory, one advantage of this ligation method may be that this reaction can initiated by the addition of required Cu(I) ions. Other exemplary mechanisms by which RNA segments may be connected include, without limitation, the use of halogens (F-, Br-, I-)/alkynes addition reactions, carbonyls/sulfhydryls/maleimide, and carboxyl/amine linkages. For example, one RNA molecule may be modified with thiol at 3′ (using disulfide amidite and universal support or disulfide modified support), and the other RNA molecule may be modified with acrydite at 5′ (using acrylic phosphoramidite), then the two RNA molecules can be connected by a Michael addition reaction. This strategy can also be applied to connecting multiple RNA molecules stepwise. Also provided are methods for linking more than two (e.g., three, four, five, six, etc.) RNA molecules to each other. Without wishing to be bound by theory, this may be useful when a desired RNA molecule is longer than about 40 nucleotides, e.g., such that chemical synthesis efficiency degrades, e.g., as noted in US20160102322A1 (incorporated herein by reference in its entirety).

By way of illustration, a tracrRNA is typically around 80 nucleotides in length. Such RNA molecules may be produced, for example, by processes such as in vitro transcription or chemical synthesis. In some embodiments, when chemical synthesis is used to produce such RNA molecules, they may be produced as a single synthesis product or by linking two or more synthesized RNA segments to each other. In embodiments, when three or more RNA segments are connected to each other, different methods may be used to link the individual segments together. Also, the RNA segments may be connected to each other in one pot (e.g., a container, vessel, well, tube, plate, or other receptacle), all at the same time, or in one pot at different times or in different pots at different times. In a non-limiting example, to assemble RNA Segments 1, 2 and 3 in numerical order, RNA Segments 1 and 2 may first be connected, 5′ to 3′, to each other. The reaction product may then be purified for reaction mixture components (e.g., by chromatography), then placed in a second pot, for connection of the 3′ terminus with the 5′ terminus of RNA Segment 3. The final reaction product may then be connected to the 5′ terminus of RNA Segment 3.

In another non-limiting example, RNA Segment 1 (about 30 nucleotides) is the target locus recognition sequence of a crRNA and a portion of Hairpin Region 1. RNA Segment 2 (about 35 nucleotides) contains the remainder of Hairpin Region 1 and some of the linear tracrRNA between Hairpin Region 1 and Hairpin Region 2. RNA Segment 3 (about 35 nucleotides) contains the remainder of the linear tracrRNA between Hairpin Region 1 and Hairpin Region 2 and all of Hairpin Region 2. In this example, RNA Segments 2 and 3 are linked, 5′ to 3′, using click chemistry. Further, the 5′ and 3′ end termini of the reaction product are both phosphorylated. The reaction product is then contacted with RNA Segment 1, having a 3′ terminal hydroxyl group, and T4 RNA ligase to produce a guide RNA molecule.

A number of additional linking chemistries may be used to connect RNA segments according to method of the invention. Some of these chemistries are set out in Table 6 of US20160102322A1, which is incorporated herein by reference in its entirety.

### Applications

By integrating coding genes into a RNA sequence template, the system can address therapeutic needs, for example, by providing expression of a therapeutic transgene in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, the RNA sequence template encodes a promotor region specific to the therapeutic needs of the host cell, for example a tissue specific promotor or enhancer. In still other embodiments, a promotor can be operably linked to a coding sequence.

In some embodiments, a system as described herein can be used to make an insertion, deletion, substitution, or combination thereof in a cell, tissue, or subject. In some embodiments, an insertion, deletion, substitution, or combination thereof, increases or decreases expression (e.g. transcription or translation) of a gene. In some embodiments, an insertion, deletion, substitution, or combination thereof, increases or decreases expression (e.g. transcription or translation) of a gene by altering, adding, or deleting sequences in a promoter or enhancer, e.g. sequences that bind transcription factors. In some embodiments, an insertion, deletion, substitution, or combination thereof alters translation of a gene (e.g. alters an amino acid sequence), inserts or deletes a start or stop codon, alters or fixes the translation frame of a gene. In some embodiments, an insertion, deletion, substitution, or combination thereof alters splicing of a gene, e.g. by inserting, deleting, or altering a splice acceptor or donor site. In some embodiments, an insertion, deletion, substitution, or combination thereof alters transcript or protein half-life. In some embodiments, an insertion, deletion, substitution, or combination thereof alters protein localization in the cell (e.g. from the cytoplasm to a mitochondria, from the cytoplasm into the extracellular space (e.g. adds a secretion tag)). In some embodiments, an insertion, deletion, substitution, or combination thereof alters (e.g. improves) protein folding (e.g. to prevent accumulation of misfolded proteins). In some embodiments, an insertion, deletion, substitution, or combination thereof, alters, increases, decreases the activity of a gene, e.g. a protein encoded by the gene.

The disclosure is directed, in part, to a method of modifying a target site in genomic DNA in a cell. In some embodiments, the method comprises contacting the cell with a system, template RNA, virus, viral-like particle, or virosome, or LNP described herein, or DNA encoding the same, thereby modifying the target site in genomic DNA in a cell.

The disclosure is directed, in part, to a method for treating a subject having a disease or condition associated with a genetic defect. In some embodiments, the method comprises administering to the subject a system, template RNA, virus, viral-like particle, or virosome, or LNP described herein, or DNA encoding the same, thereby treating the subject having a disease or condition associated with a genetic defect. In some embodiments, the disease or condition associated with a genetic defect is an indication listed in any of Tables 9-12 of International Application PCT/US2021/020948 filed Mar. 4, 2021, which is herein incorporated by reference in its entirety including said tables, and/or wherein the genetic defect is a defect in a gene listed in any of said Tables 9-12 therein. In some embodiments, the subject is a human subject.

In some embodiments, a system or template nucleic acid (e.g., template RNA) described herein can be prepared (e.g., prepared for administration or delivery to a subject, tissue, or cell) as a lipid nanoparticle. The disclosure is also directed, in part, to lipid nanoparticles (LNPs) comprising the system or template nucleic acid (e.g., template RNA), or DNA encoding the system or template RNA.

In some embodiments, a system or template nucleic acid (e.g., template RNA) described herein can be prepared (e.g., prepared for administration or delivery to a subject, tissue, or cell) as a virus, viral-like particle, or virosome. The disclosure is also directed, in part, to a virus, viral-like particle, or virosome comprising the system or template nucleic acid (e.g., template RNA), or DNA encoding the system or template RNA. In some embodiments, the virus, viral-like particle, or virosome comprises an adeno-associated virus (AAV) capsid protein. In some embodiments, the virus, viral-like particle, or virosome is an AAV. The disclosure is also directed, in part, to reaction mixtures comprising a system or template nucleic acid (e.g., template RNA) described herein and a cell (e.g., a cell from a cell line or a cell from a subject, e.g., a human cell). The disclosure is further directed, in part, to reaction mixtures comprising a system or template nucleic acid (e.g., template RNA) described herein and DNA (e.g., genomic DNA or a vector) comprising a target site (e.g., a target site that is the target of the system or template RNA).

The disclosure is also directed, in part, to kits comprising a system, template nucleic acid (e.g., template RNA), or reaction mixture described herein, and instructions for using instructions for using the system, template RNA, or reaction mixture. In some embodiments, a kit further comprises a cell (e.g., a cell from a cell line or a cell from a subject, e.g., a human cell) or DNA (e.g., genomic DNA or a vector) comprising a target site (e.g., a target site that is the target of the system or template RNA).

### EXAMPLES

### Example 1: Preventing Reverse Transcriptase Readthrough and Integration of Downstream Sequences

This example describes the use of an improved template nucleic acid comprising an additional cis-regulatory feature for precise termination of reverse transcription. Specifically, the template design is added with 1) a reverse transcriptase (RT) termination motif (an RT terminator) or 2) an artificially stabilized RNA hairpin loop. During HIV-1 reverse transcription, the plus-strand of viral DNA is synthesized as two discrete segments. It was shown by Charneau et al. J Mol Biol 241(5):651-662 (1994) that synthesis of the upstream segment terminates at the center of the genome after an 88 or 98 nucleotide strand displacement of the downstream segment, initiated at the central polypurine tract. In vitro reconstitution using only purified reverse transcriptase with appropriate DNA hybrids gave rise to efficient and accurate termination, while mutation of the sequence immediately upstream of the termination sites almost completely abolished termination both in infected cells and in vitro. These two termination sites of the central termination sequence (CTS) are known as called Ter1 and Ter2.

As described in bacteria, RT termination may be triggered a GC-rich dyad repeat that forms a hairpin structure facilitated by RNA polymerase pausing during transcription (Ray-Soni et al. Annual Rev of Biochem 85:319-347 (2016). The hairpin loop may destabilize specific binding interactions between polymerase and the nascent RNA (Wilson et al. Proc Natl Acad Sci USA 92:8793-8797 (1995) or cause ribosome-stalling resulting in the accumulation of truncated polypeptides (Yan et al. Cell 160(5):870-881 (2015). Inclusion of a hairpin-forming region between the template and the scaffold may stimulate RT termination before the scaffold is reverse transcribed.

In this example, an exemplary template RNA follows a structure of
(1) gRNA spacer
(2) gRNA scaffold
(3) Heterologous object sequence
(4) 3′ target homology
   Given the orientation of the construct, it is possible that the RT reads from (3) into (2), integrating undesirable sequences and features. Thus, either the CTS from HIV-1 or a stabilized hairpin loop is placed between (3) and (2) to ensure proper termination.

A system comprising a polypeptide (e.g., comprising an RT domain) and a template RNA encoding a GFP reporter either (a) as is; or (b) with the HIV-1 CTS, is transfected into HEK293T cells. After three days, genomic DNA is harvested for analysis. To analyze the integration events, primers flanking the genomic target site are used to amplify across the locus. Amplicons are analyzed via Long-read amplicon sequencing (e.g., PacBio). The incorporation of bases from region (4) of the template RNA may be indicative of a lack of appropriate termination. In some embodiments, the template RNA with the added termination structure does not incorporate non-desired portions of the template RNA, which could represent unnecessary addition of exogenous sequence into genomic DNA. In some embodiments, the template RNA with the added termination structure incorporates non-desired portions of the template RNA (e.g., which could include unnecessary exogenous sequence) at a reduced rate e.g., as compared to the standard template.

### Example 2: Reducing Secondary Structure of the Reverse Transcriptase Template to Improve its Integration

This example describes the improvement of the template nucleic acid component of a gene editing system designed to treat a repeat expansion disease by rewriting a normal number of repeats into the locus. More specifically, the polypeptide and template RNA are delivered to cells to rewrite CAG repeats in HTT as per the template RNA heterologous object sequence to treat Huntington Disease.

Healthy humans tend to carry between 10 and 35 CAG repeats within the huntingtin gene (HTT), while those with Huntington Disease may possess between 36 to greater than 120 repeats. In order to address pathogenic loci with an expanded number of repeats, a template RNA was designed to work with a system to replace the expanded number of trinucleotide repeats (e.g., 100 repeats) with a reference number of repeats, specifically here using the human genome reference repeat locus (NC 000004.12:3,074,877-3,074,939). An exemplary template RNA was first designed, comprising the sequence

(SEQ ID NO: 5)
(1)GGCGGCUGAGGAAGCUGAGG(2)GUUUUAGAGCUA
GAAAUAGCAAGUUAAAAUAAGGCUAGUCCGUUAUCAAC
UUGAAAAAGUGGGACCGAGUCGGUCC(3)AGUCCCUCA
AGUCCUUCCAGCAGCAGCAGCAGCAGCAGCAGCAGCAG
CAGCAGCAGCAGCAGCAGCAGCAGCAGCAACAGCCGCC
ACCGCCGCCGCCGCCGCCGCCGCCUCCU(4)CAGCUUC
CUCAG,

where numbers are used to delineate the modules of the template in the order (5′-3′): (1) gRNA spacer, (2) gRNA scaffold, (3) heterologous object sequence, (4) 3′ homology priming domain, with the repeat correction being encoded in (3). The CAG repeat region is followed by a short repeat region encoding for 11 proline residues (8 residues being encoded by CCG triplets) and this region was included in (3) to place (4) in a more unique region to prevent mispriming. An exemplary gRNA for providing a second nick as described in embodiments of this system comprises the spacer sequence CGCTGCACCGACCGTGAGTT (SEQ ID NO: 6) and directs a Cas9 nickase to nick the second strand of the target site within the homologous region. In some embodiments, this second nick improves the efficiency of the editing by the system.

The tool RNAstructure (Bellaousov et al. Nucleic Acids Res 41:W471-W474 (2013)) was used to analyze the initial template nucleic acid sequence, and extensive secondary structure was observed in the heterologous object sequence, owing to base pairing between GC dinucleotides (FIG. 1). The molecule yielded a predicted folding free energy of −80.6 kcal/mol. Secondary structure could impact the ability of the reverse transcriptase domain of a polypeptide, so a second template was designed from the first with an effort to reduce these structures. Specifically, the heterologous object sequence was extracted and the repeat regions, as described above, were run through mRNAoptimiser, a tool designed to alter (e.g. increase, decrease) the secondary structures of mRNA without changing the amino acid sequence of the encoded protein (Gaspar et al. Nucleic Acids Res 41(6):e73 (2013)). Since the repeat region addressed here is in the coding region of the HTT gene, it is desirable to maintain the coding potential of this region. Placing the improved region back into the template RNA yielded the sequence, from 5′ to 3′ (number indications are as described in Example 1):

(SEQ ID NO: 7)
(1)GGCGGCUGAGGAAGCUGAGG(2)GUUUUAGAGCUAG
AAAUAGCAAGUUAAAAUAAGGCUAGUCCGUUAUCAACUU
GAAAAAGUGGGACCGAGUCGGUCC(3)AGUCCCUCAAGU
CCUUCCAACAACAACAACAACAACAACAACAACAACAAC
AACAACAACAACAACAACAACAACAACAACCACCCCCAC
CACCACCACCACCACCACCCCCA(4)CAGCUUCCUCAG,

which yields a new folding free energy of −45.0, indicating the loss of secondary structure from the molecule.

To assay template nucleic acids for integration, an mRNA encoding a polypeptide as described herein (e.g., comprising an RT domain) is co-transfected with template RNA into HEK293 cells containing an expansion of the CAG trinucleotide repeat in HTT (Morozova et al. PLoS One 13(10):e0204735 (2018)). After three days, genomic DNA is extracted and analyzed for the efficiency of correct editing. In brief, primers flanking the HTT locus are used to amplify the repeat region, and amplicons are analyzed by long-read sequencing (e.g., PacBio). In some embodiments, an improved template RNA demonstrates a higher fraction of integration events as indicated by a higher fraction of HTT loci containing 21 or fewer repeats. In some embodiments, an improved template RNA leads to a higher processivity of the polypeptide, as evidenced by analyzing the fraction of complete integrations (21 trinucleotide repeats) and truncated integrations (less than 21 trinucleotide repeats).

### Example 3: Reducing Secondary Structure of the Reverse Transcriptase Template to Improve its Integration

This example describes the improvement of the template nucleic acid component of a gene editing system designed to treat OTC deficiency. More specifically, the polypeptide and template RNA are delivered to cells to write an expression cassette encoding OTC into the AAVS1 locus.

To target the polypeptide to the AAVS1 safe harbor locus, a Cas9-RT fusion (e.g., nickase Cas9 fused to the RT domain of the retrotransposon R2Tg) is used in combination with a template RNA comprising a gRNA targeting the AAVS1 site, e.g., GGGGCCACTAGGGACAGGAT (SEQ ID NO: 8). A template RNA was first created containing the following components:
(1) 100 nt homology to AAVS1 site, upstream
(2) 5′ UTR of R2Tg
(3) Heterologous object sequence
(4) 3′ UTR of R2Tg
(5) 100 nt homology to AAVS1 site, downstream
   Where (3) comprises a simple OTC expression cassette in which the OTC coding sequence is operably linked to the CMV promoter. To analyze secondary structure of the OTC coding sequence, RNAstructure (Bellaousov et al. Nucleic Acids Res 41:W471-W474 (2013)) was employed on the mRNA coding sequence, defined here as. Folding predictions from RNAstructure revealed a free folding energy of −408.1.

Secondary structure can impact the activity of the reverse transcriptase domain of a polypeptide as described herein. Additional templates are derived from the first with an effort to reduce these structures. Specifically, the coding sequence is extracted and run through either a codon-optimizer (e.g., Geneious Prime), designed to maximize expression by codon utilization, or mRNAoptimiser, a tool designed to alter (e.g. increase, decrease) the secondary structures of mRNA without changing the amino acid sequence of the encoded protein (Gaspar et al. Nucleic Acids Res 41(6):e73 (2013)). As compared to the initial folding free energy of −408.1 kcal/mol, the codon optimization approach yielded a slightly improved −341.4 kcal/mol, while the structure optimization approach yielded a much improved −150.1 kcal/mol.

To assay the three templates comprising various optimizations of OTC for integration, a mRNA encoding a polypeptide as described herein (e.g., comprising an RT domain) is co-transfected into HEK293T cells with a template RNA. After three days, genomic DNA is extracted and analyzed for the efficiency of integration of the OTC cassette. To look at efficiency of integration, a ddPCR assay is performed with a primer pair flanking the 3′ junction of the integration (e.g., one primer anneals to the integration sequence and one primer anneals to the flanking AAVS1 genomic sequence. By comparing to an internal reference for each condition (e.g., RPP30), the fraction of cells containing an insert can be approximated and used as a measure of efficiency. In some embodiments, a structure-optimized template RNA yields a higher efficiency of integration.

To analyze processivity, primers flanking the AAVS1 integration site are used to amplify the target region, and amplicons are analyzed by long-read sequencing (e.g., PacBio). Analysis of processivity is performed by looking at complete integrations (e.g., containing the full OTC expression cassette) as compared to partial integrations (e.g., containing at least part of the template sequence). In some embodiments, a structure-optimized template demonstrates an increased fraction of complete integration events as compared to total integration events.

TABLE 7
Exemplary template RNAs comprising coding
sequence of OTC and associated folding
free energy. The wild-type OTC mRNA is
derived from NCBI GeneID: 5009. This
sequence was either codon-optimized
(Geneious Prime) or improved to minimize
folding free energy (mRNAoptimiser). All
structures were analyzed for folding free
energy by RNAstructure.

| | | Folding |
|---|---|---|
| | | Free |
| | | Energy |
| Variant | Sequence | (kcal/mol) |
| OTC | AUGCUGUUUAAUCUGAGGAUCCUGUUAAACAAUGCAGC | −408.1 |
| mRNA | UUUUAGAAAUGGUCACAACUUCAUGGUUCGAAAUUUUC | |
| | GGUGUGGACAACCACUACAAAAUAAAGUGCAGCUGAAG | |
| | GGCCGUGACCUUCUCACUCUAAAAAACUUUACCGGAGA | |
| | AGAAAUUAAAUAUAUGCUAUGGCUAUCAGCAGAUCUGA | |
| | AAUUUAGGAUAAAACAGAAAGGAGAGUAUUUGCCUUU | |
| | AUUGCAAGGGAAGUCCUUAGGCAUGAUUUUUGAGAAAA | |
| | GAAGUACUCGAACAAGAUUGUCUACAGAAACAGGCUUU | |
| | GCACUUCUGGGAGGACAUCCUUGUUUUCUUACCACACA | |
| | AGAUAUUCAUUUGGGUGUGAAUGAAAGUCUCACGGACA | |
| | CGGCCCGUGUAUUGUCUAGCAUGGCAGAUGCAGUAUUG | |
| | GCUCGAGUGUAUAAACAAUCAGAUUUGGACACCCUGGC | |
| | UAAAGAAGCAUCCAUCCCAAUUAUCAAUGGGCUGUCAG | |
| | AUUUGUACCAUCCUAUCCAGAUCCUGGCUGAUUACCUC | |
| | ACGCUCCAGGAACACUAUAGCUCUCUGAAAGGUCUUAC | |
| | CCUCAGCUGGAUCGGGGAUGGGAACAAUAUCCUGCACU | |
| | CCAUCAUGAUGAGCGCAGCGAAAUUCGGAAUGCACCUU | |
| | CAGGCAGCUACUCCAAAGGGUUAUGAGCCGGAUGCUAG | |
| | UGUAACCAAGUUGGCAGAGCAGUAUGCCAAAGAGAAUG | |
| | GUACCAAGCUGUUGCUGACAAAUGAUCCAUUGGAAGCA | |
| | GCGCAUGGAGGCAAUGUAUUAAUUACAGACACUUGGAU | |
| | AAGCAUGGGACAAGAAGAGGAGAAGAAAAAGCGGCUCC | |
| | AGGCUUUCCAAGGUUACCAGGUUACAAUGAAGACUGCU | |
| | AAAGUUGCUGCCUCUGACUGGACAUUUUUACACUGCUU | |
| | GCCCAGAAAGCCAGAAGAAGUGGAUGAUGAAGUCUUUU | |
| | AUUCUCCUCGAUCACUAGUGUUCCCAGAGGCAGAAAAC | |
| | AGAAAGUGGACAAUCAUGGCUGUCAUGGUGUCCCUGCU | |
| | GACAGAUUACUCACCUCAGCUCCAGAAGCCUAAAUUUU | |
| | GA (SEQ ID NO: 9) | |
| Codon | AUGCUGUUUAAUUUGCGGAUCUUGCUCAACAAUGCCGC | −341.4 |
| optimized | UUUUCGCAACGGACACAACUUCAUGGUGCGUAACUUCA | |
| | GAUGCGGCCAGCCGCUGCAAAACAAAGUCCAGCUGAAG | |
| | GGCCGCGACCUUUUGACCCUGAAGAACUUCACUGGCGA | |
| | AGAGAUCAAGUACAUGUUGUGGCUGUCUGCCGAUCUGA | |
| | AAUUCCGUAUUAAACAAAAGGGUGAGUACCUGCCUUUG | |
| | CUGCAGGGAAAGAGUCUGGGUAUGAUUUUUGAGAAGCG | |
| | CUCCACCAGGACGCGGCUCAGCACCGAAACAGGAUUCG | |
| | CUCUCCUGGGCGGGCACCCUUGCUUCCUCACCACACAG | |
| | GACAUCCACCUCGGCGUGAACGAGUCCCUGACUGACAC | |
| | UGCGCGCGUCCUGUCUUCCAUGGCCGAUGCUGUGCUGG | |
| | CUCGUGUGUACAAACAGAGCGACCUGGAUACUUUGGCU | |
| | AAAGAGGCCUCAAUCCCUAUUAUCAAUGGUCUUUCCGA | |
| | CCUUUAUCAUCCGAUCCAGAUCUUGGCUGACUAUCUGA | |
| | CUCUGCAGGAACACUAUUCUUCCCUGAAAGGCCUCACC | |
| | UUGUCAUGGAUUGGCGACGGCAAUAACAUCCUUCACAG | |
| | CAUCAUGAUGAGCGCCGCGAAGUUCGGGAUGCAUCUGC | |
| | AAGCGGCUACGCCCAAGGGCUACGAGCCCGAUGCCAGU | |
| | GUGACCAAGCUGGCUGAGCAGUAUGCCAAGGAAAAUGG | |
| | AACAAAGCUUCUCCUGACCAACGAUCCACUCGAAGCGG | |
| | CUCACGGGGGUAACGUGCUGAUUACUGACACAUGGAUC | |
| | UCCAUGGGGCAGGAGGAAGAGAAGAAAAAGCGCCUGCA | |
| | GGCGUUUCAGGGGUACCAGGUGACAAUGAAGACCGCUA | |
| | AGGUCGCCGCUUCCGAUUGGACUUUCUUGCAUUGCCUG | |
| | CCCAGAAAGCCCGAAGAGGUUGAUGACGAGGUGUUUUA | |
| | CUCCCCUCGCUCCCUGGUGUUCCCCGAGGCCGAGAAUC | |
| | GCAAAUGGACAAUUAUGGCUGUGAUGGUCUCUCUCCUG | |
| | ACCGACUACUCCCCUCAGCUCCAGAAGCCAAAGUUCUA | |
| | A (SEQ ID NO: 10) | |
| Structure | AUGCUCUUCAAUCUAAGAAUACUACUAAACAACGCAGC | −150.1 |
| optimized | AUUCAGAAAUGGACACAACUUUAUGGUAAGAAACUUCC | |
| | GAUGUGGACAACCACUACAAAACAAAGUACAACUAAAA | |
| | GGAAGAGACCUAUUAACACUCAAAAACUUCACAGGUGA | |
| | AGAAAUAAAAUACAUGCUAUGGCUAUCCGCAGAUUUAA | |
| | AAUUCAGAAUAAAACAAAAAGGAGAAUACCUACCUCUU | |
| | CUACAAGGAAAAUCACUAGGAAUGAUAUUCGAAAAAAG | |
| | AUCAACACGAACAAGACUAAGCACCGAAACCGGAUUCG | |
| | CACUACUAGGAGGACAUCCCUGCUUCCUUACAACUCAA | |
| | GACAUCCAUUUAGGAGUAAACGAAUCACUAACCGACAC | |
| | AGCAAGAGUAUUAAGCUCAAUGGCAGAUGCAGUACUUG | |
| | CAAGAGUAUACAAACAAUCAGACCUAGACACACUAGCA | |
| | AAAGAAGCAUCAAUCCCAAUAAUAAACGGACUUUCAGA | |
| | CUUAUACCAUCCAAUACAAAUUUUAGCAGACUACUUAA | |
| | CCUUACAAGAACACUACUCCUCACUGAAAGGACUUACA | |
| | CUAUCCUGGAUAGGAGACGGAAACAACAUCCUACACUC | |
| | AAUAAUGAUGUCAGCAGCAAAAUUCGGAAUGCACCUAC | |
| | AAGCAGCCACACCAAAAGGAUACGAACCAGAUGCUUCA | |
| | GUAACAAAACUAGCAGAACAAUACGCAAAAGAAAACGG | |
| | AACUAAACUAUUAUUAACAAACGACCCACUCGAAGCAG | |
| | CACACGGAGGAAACGUACUCAUCACAGACACAUGGAUA | |
| | UCAAUGGGACAAGAAGAAGAAAAAAAAAAAAGACUACA | |
| | AGCAUUCCAAGGAUACCAAGUAACAAUGAAAACAGCAA | |
| | AAGUCGCAGCAUCCGACUGGACAUUCUUACACUGUCUA | |
| | CCACGAAAACCAGAAGAAGUCGACGACGAAGUAUUCUA | |
| | UAGUCCAAGAUCAUUAGUUUUCCCAGAAGCAGAAAACA | |
| | GAAAAUGGACCAUAAUGGCAGUAAUGGUAUCACUACUA | |
| | ACAGAUUACUCACCCCAACUACAAAAACCAAAAUUUUA | |
| | A (SEQ ID NO: 11) | |

### Example 4: Preventing Reverse Transcriptase Readthrough and Integration of Downstream Sequences Using Streptavidin-Biotin Spacer

This example describes the use of an improved template nucleic acid involving a non-covalently bound spacer feature for precise termination of reverse transcription. Specifically, a biotin-streptavidin-biotin motif is added between the template and the scaffold regions to maintain their connectivity and simultaneously to impede undesired readthrough of RT into the scaffold region.

The affinity between streptavidin (Sty) and biotin is one of the strongest non-covalent interactions known. With the advances in solid phase synthesis of oligonucleotides, biotinylated oligos have become more accessible nowadays leading to a plethora of applications in the field of biotechnology. Biotin-Sty-labeled oligonucleotides have been used by Raney et al. Methods 23:149-159 (2001) (doi.org/10.1006/meth.2000.1116) as a “protein block” to prevent translocation of helicase enzyme during the unwinding of double-stranded nucleic acids.

In this example, an exemplary template RNA follows, from 5′ to 3′, a structure of
(1) gRNA spacer
(2) gRNA scaffold terminated with biotin
(3) Streptavidin
(4) Heterologous object sequence terminated with biotin
(5) 3′ target homology
   An RT using this template RNA uses (5) as a primer binding site, and proceeds to reverse-transcribe (4). The presence of (3) will increase the likelihood that the RT will terminate reverse transcription at that point, because (3) lacks molecular structure the RT requires to traverse a polynucleotide. Consequently the RT will be prevented from continuing reverse transcription into (2). The biotin-streptavidin-biotin therefore is placed between (4) and (2) to ensure proper termination.

FIG. 7B shows a system having a polypeptide as described herein and a template RNA encoding a GFP reporter with the biotin-streptavidin-biotin motif. The template RNA containing the spacer modification is synthesized by incubating oligos A and B and streptavidin. Oligo A sequence (5′ 4 3′) contains (1) and (2) including a biotin modification hanging off the 3′ end of the molecule. Oligo B sequence (5′ 4 3′) contains (5) and (4) including a biotin modification on the 5′ end of the molecule. The system is transfected into HEK293T cell which after three days, genomic DNA is harvested for analysis. To analyze the integration events, primers flanking the genomic target site are used to amplify across the locus. Amplicons are analyzed via Long-read amplicon sequencing (e.g., PacBio). The incorporation of bases from region (2) of the template RNA may be indicative of a lack of appropriate termination. In some embodiments, the template RNA with the added spacer motif structure does not incorporate non-desired portions of the template RNA, which could represent unnecessary addition of exogenous sequence into genomic DNA. In some embodiments, the template RNA with the added termination structure incorporates non-desired portions of the template RNA (e.g., which could include unnecessary exogenous sequence) at a reduced rate e.g., as compared to the standard template.

### Example 5: Preventing Reverse Transcriptase Readthrough and Integration of Downstream Sequences Using Triazole or PEG Spacer

This example describes the use of an improved template nucleic acid involving a covalently attached spacer feature for precise termination of reverse transcription. Specifically, the template and the scaffold regions are connected via triazole or polyethylene glycol (PEG) spacer of varying carbon chain lengths to impede undesired readthrough of RT into the scaffold region.

In this example, an exemplary template RNA follows a 5′ to 3′ structure of
(1) gRNA spacer
(2) gRNA scaffold
(3) Triazole or PEG spacer
(4) Heterologous object sequence
(5) 3′ target homology
   An RT using this template RNA uses (5) as a primer binding site, and proceeds to reverse-transcribe (4). The presence of a triazole or TEG spacer will increase the likelihood that the RT will terminate reverse transcription at that point, because it lacks the nucleotide backbone required for transcription. Consequently the RT will be prevented from continuing reverse transcription into (2). The triazole or PEG spacer therefore is placed between (2) and (4) to ensure proper termination.

A system having a polypeptide as described herein (e.g., comprising an RT domain) and a template RNA encoding a GFP reporter either (a) without; or (b) with Triazole or PEG spacer is generated. The template RNA containing the triazole modification is synthesized by Click chemistry with one oligo containing the azide functionality while the other oligo contains alkyne functionality. Template RNA containing the PEG spacer motif is synthesized by conventional Solid Phase Synthesis using phosphoramidite chemistry. The system is transfected into HEK293T cell which after three days, genomic DNA is harvested for analysis. To analyze the integration events, primers flanking the genomic target site are used to amplify across the locus. Amplicons are analyzed via Long-read amplicon sequencing (e.g., PacBio). The incorporation of bases from region (2) of the template RNA may be indicative of a lack of appropriate termination. In some embodiments, the template RNA with the added linker or spacer motif does not incorporate non-desired portions of the template RNA, which could represent unnecessary addition of exogenous sequence into genomic DNA. In some embodiments, the template RNA with the added linker or spacer motif incorporates non-desired portions of the template RNA (e.g., which could include unnecessary exogenous sequence) at a reduced rate e.g., as compared to the standard template.

Other spacers can be used, such as those listed in Table 8.

TABLE 8
Internal Spacers

| | | Mode |
|---|---|---|
| | | of |
| Modifi- | | Attach- |
| cation | Structure | ment |
| triazole | 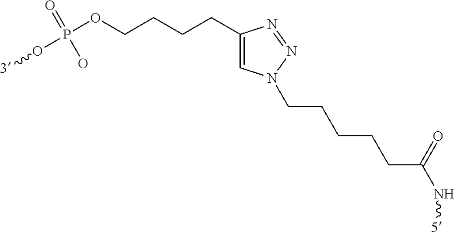 | Chem- ical reac- tion (Click) |
| DBCO- triazole | 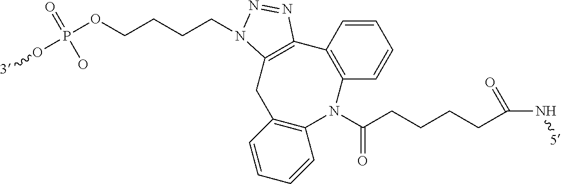 | Chem- ical reac- tion (Click) |
| DBCO- TEG- triazole | 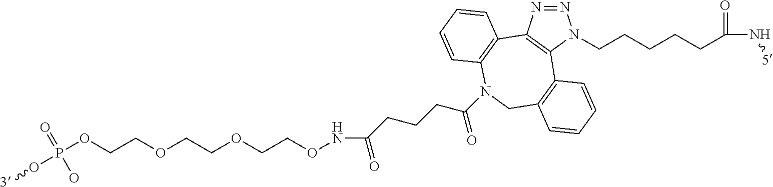 | Chem- ical reac- tion (Click) |
| Biotin- Strepta- vidin- Biotin | 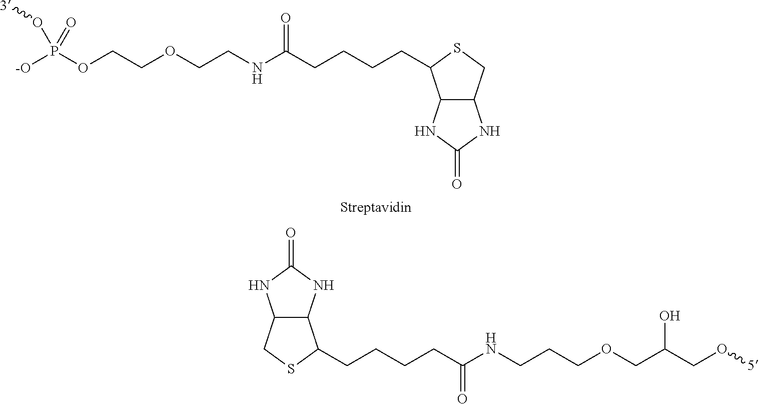 | Affin- ity |

### Example 6: Use of Dual Template gRNA to Increase Rewriting Efficiency

A cis-gene writing system that employs a single guide RNA called tgRNA that encodes the intended edit at a desired locus incorporates the edit on only one DNA strand. The edit is likely incorporated into the second DNA strand via the mismatch repair pathway. However, mismatch repair can also remove the edit from the first strand. The rewriting efficacy can be enhanced in some cases using a second nicking gRNA which can trigger mismatch repair and incorporate the edit on both DNA strands resulting in enhanced efficacy. However, use of a second nicking gRNA can also increase the incorporation of unintended edits, also called insertions and deletions (indels).

An alternative strategy to enhance precise editing is by using two template gRNAs in an anti-parallel orientation. Both the template gRNAs encode the same edit but on opposite DNA strands. Therefore, the intended edit is incorporated in both DNA strands, resulting in higher editing efficiency overall. This strategy is particularly useful for long insertions and deletions.

An example of a pathogenic mutation requiring correction via long deletion is Huntington Disease. Healthy humans tend to carry between 10 and 35 CAG repeats within the huntingtin gene (HTT), while those with Huntington Disease may possess between 36 to greater than 120 repeats. An exemplary tgRNA was first designed, having the sequence:

(SEQ ID NO: 12)
(Spacer)GGCGGCUGAGGAAGCUGAGG(Scaffold)
GUUUUAGAGCUAGAAAUAGCAAGUUAAAAUAAGGCUA
GUCCGUUAUCAACUUGAAAAAGUGGGACCGAGUCGGU
CC(Template)AGUCCCUCAAGUCCUUCCAGCAGCA
GCAGCAGCAGCAGCAGCAGCAGCAGCAGCAGCAGCAG
CAGCAGCAGCAGCAACAGCCGCCACCGCCGCCGCCGC
CGCCGCCGCCUCCU(Priming)CAGCUUCCUCAG

This tgRNA, when acting on the HTT locus, will cause deletion from the antisense strand all but the 19 repeats complementary to the CAG repeats represented in the template region. Once this occurs, the two strands of the DNA in this region of the HTT gene will no longer match, and correction by mismatch repair could result in either excision of the same complementary repeats from the sense strand (desired result) or restoration of the excised repeats to the antisense strand (undesired result). To reduce the chance of the undesired restoration, a second, anti-parallel, tgRNA designed to incorporate the same edit (deletion of pathogenic repeats) in the opposite strand is included:

(SEQ ID NO: 13)
(Spacer)GACCCUGGAAAAGCUGAUGA(Scaffold)
GUUUUAGAGCUAGAAAUAGCAAGUUAAAAUAAGGCUA
GUCCGUUAUCAACUUGAAAAAGUGGGACCGAGUCGGU
CC(Template)CUGAGGAAGCUGAGGAGGCGGCGGC
GGCGGCGGCGGCGGUGGCGGCUGUUGCUGCUGCUGCU
GCUGCUGCUGCUGCUGCUGCUGCUGCUGCUGCUGCUG
CUGCUGCUGGAAGGACUUGAGGGACUCGAAGGCCUUC
A(Priming)UCAGCUUUUCCAG

To assay template nucleic acids for integration, a Rewriter mRNA is co-transfected with template RNA into HEK293 cells containing an expansion of the CAG trinucleotide repeat in HTT (Morozova et al. PLoS One 13(10):e0204735 (2018)) [https://doi.org/10.1371/journal.pone.0204735]. After three days, genomic DNA is extracted and analyzed for the efficiency of correct editing. In brief, primers flanking the HTT locus are used to amplify the repeat region, and amplicons are analyzed by long-read sequencing (e.g., PacBio) and/or gel electrophoresis to detect a size-change in the amplified fragment. A successful deployment of dual template gRNAs demonstrates a higher fraction of integration events as indicated by a higher fraction of HTT loci containing 19 or fewer CAG repeats.

### Example 7: Improving Template Guide RNA Stability with Chemical Modifications

In this example, a sterically bulky chemical modification to the 3′ end of a tgRNA can be made to improve stability. The template and priming components of a tgRNA are relatively susceptible to degradation by exonucleases present in cells because they are relatively unprotected by polypeptide, compared to the spacer and scaffold components. 3′ chemical modification can provide additional protection to the 3′ region of the tgRNA.

Sterically bulky chemical modifications that can be added to the 3′ end are listed in Table 9 and these are predicted to prevent cleavage by 3′ exonucleases.

TABLE 9
5′ and 3′ Sterically bulky protecting groups

| Modification | Structure |
|---|---|
| 5′-Biotin | 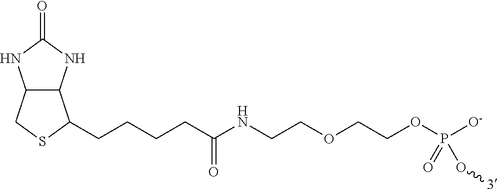 |
| 3′-Biotin | 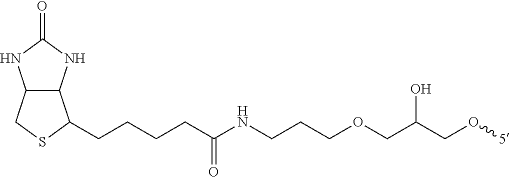 |
| 5′-Biotin- TEG | 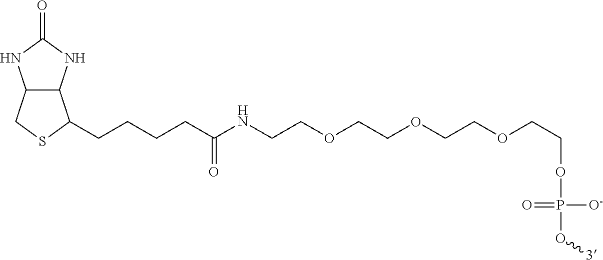 |
| 3′-Biotin- TEG | 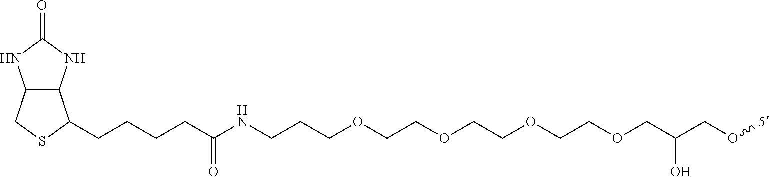 |
| 5′-Dual- Biotin | 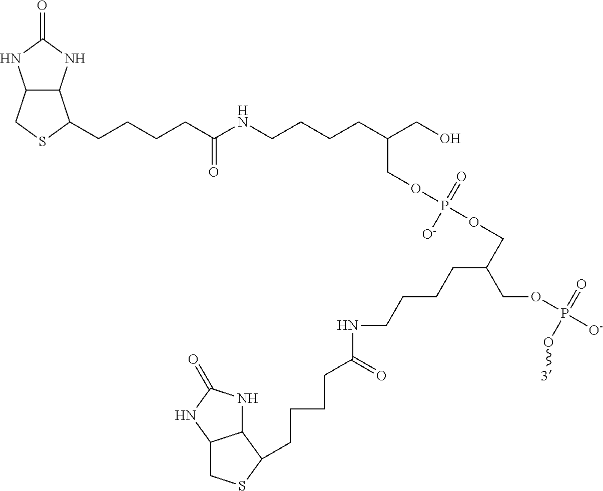 |
| Cholesterol- TEG | 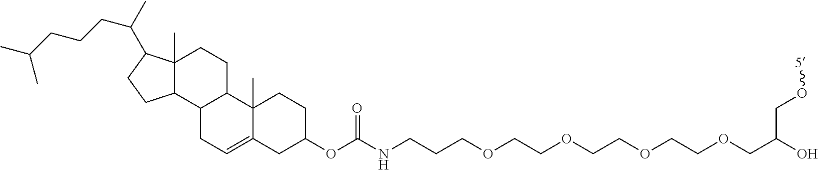 |

Fah encodes the liver enzyme fumarylacetoacetate hydrolase. In model cell lines and animal models, the Fah gene is disrupted, for example, the mouse model Fah5981SB (PMID: 24681508) contains a single nucleotide splicing mutation (G->A) in exon 8 leading to FAH deficiency. To correct the mutation, we designed a tgRNA called FAH1 which encodes a single nucleotide change that corrects the splicing mutation (A→G correction encoded by C in the sequence below).

(SEQ ID NO: 14)
(spacer)GGAUGGUCCUCAUGAACGAC(scaffold)
GUUUUAGAGCUAGAAAUAGCAAGUUAAAAUAAGGCUAG
UCCGUUAUCAACUUGAAAAAGUGGCACCGAGUCGGUGC
(template)AUUAC*C*GCUCCAGUC(priming)GU
UCAUGAGGACC

In one example, tgRNAs were designed that contain sterically bulky chemical modification on the 3′ end:

(SEQ ID NO: 15)
(spacer)GGAUGGUCCUCAUGAACGAC(scaffold)
GUUUUAGAGCUAGAAAUAGCAAGUUAAAAUAAGGCUA
GUCCGUUAUCAACUUGAAAAAGUGGCACCGAGUCGGU
GC(template)AUUAC*C*GCUCCAGUC(priming)
GUUCAUGAGGACC-B

Here B refers to any bulky modification from Table 9.

### Example 8: Improving Template Guide RNA Stability with Terminal Sequence

In this example, stable RNA structures can be appended on the 3′ end of the tgRNA to resist exonuclease cleavage. G-quadruplexes have previously been shown to improve Cas9 gRNA stability resulting in increased indel formation (doi:10.1039/c7cc08893k). Xrn1 resistant RNAs (xrRNAs) are natural sequences found in the untranslated regions (UTRs) of flaviviruses that confer increased exoribonuclease resistance to their genomes (doi:10.1038/s41467-017-02604-y). These xrRNAs can also appended on the 3′ end of the tgRNAs for increased exoribonuclease protection.

A G-quadruplex sequence may be added to the 3′ end of the tgRNA to protect from exonuclease degradation:

(SEQ ID NO: 16)
(spacer)GGAUGGUCCUCAUGAACGAC(scaffold)
GUUUUAGAGCUAGAAAUAGCAAGUUAAAAUAAGGCUA
GUCCGUUAUCAACUUGAAAAAGUGGCACCGAGUCGGU
GC(template)AUUAC*C*GCUCCAGUC(priming)
GUUCAUGAGGACC(G-quadruplex)ACAUCAGGU
GGUGGUGG

To assay designed tgRNAs, a Rewriter mRNA is co-transfected with template RNA into mouse hepatocytes derived from the mouse model Fah5981SB (PMID: 24681508). After three days, genomic DNA is extracted and analyzed for the efficiency of correct editing. In brief, primers flanking the Fah locus are used to amplify locus, and amplicons are analyzed by short-read sequencing (e.g. MiSeq).

Both the tgRNAs with protected 3′ end are expected to increase exoribonuclease resistance of the Fah tgRNA resulting in higher rewriting efficiency as shown by higher fraction reads with correct A→G mutation.

### Example 9: Improving Rewriting Performance by Disrupting Spacer: Priming Annealing Through Synonymous Mutations

Because a tgRNA includes a primer component that is complementary to the spacer component, there is increased risk on secondary structure developing in the tgRNA that may interfere with both loading into the Cas9 (lowering nicking activity) and/or RT read through the entire template.

In this example, disruption of the annealing between the spacer and primer components may be facilitated by introducing sequence mutations in the priming region of the tgRNA.

Fah model cell lines may be used. Fah encodes the liver enzyme fumarylacetoacetate hydrolase. In model cell lines and animal models, the Fah gene is disrupted, for example, the mouse model Fah5981SB (PMID: 24681508) contains a single nucleotide splicing mutation (G->A) in exon 8 leading to FAH deficiency. To correct the mutation, tgRNA, FAH1 was designed to encode a single nucleotide change that corrects the splicing mutation (A→G correction encoded by C in the sequence below).

(SEQ ID NO: 17)
(spacer)GGAUGGUCCUCAUGAACGAC(scaffold)
GUUUUAGAGCUAGAAAUAGCAAGUUAAAAUAAGGCUAG
UCCGUUAUCAACUUGAAAAAGUGGCACCGAGUCGGUGC
(template)AUUAC*C*GCUCCAGUC(priming)GU
UCAUGAGGACC

The spacer and priming region form a stable duplex as predicted by the RNAcofold web server (rna.tbi.univie.ac.at/cgi-bin/RNAWebSuite/RNAcofold.cgi). The predicted free energy of this interaction is −24.14 kcal/mol. Mutations can be introduced in the priming region to disrupt this interaction as shown in the table below:

| | | Free Energy of |
|---|---|---|
| | | spacer: |
| | Priming | priming |
| Name | sequence | interaction |
| FAH-1 without | GUUCAUGAGGACC | −24.14 |
| priming | (SEQ ID | kcal/mol |
| mutation | NO: 18) | |
| mutation 1 | GUUCAUGAGAACC | −17.28 |
| | (SEQ ID | kcal/mol |
| | NO: 10) | |
| mutation 2 | GUUCAUUAGAACC | −10.97 |
| | (SEQ ID | kcal/mol |
| | NO: 18) | |
| mutation 3 | GUUCAUUUAAACC | −8.5 |
| | (SEQ ID | kcal/mol |
| | NO: 21) | |

The tgRNAs comprising the priming mutations may be tested in HEK293T model cell line by nucleofection. Briefly, 5 uM of tgRNA and 1 ug of ReWriter polypeptide mRNA (1 ug) can be mixed with 250,000 cells and nucleofected using the Lonza Nucleofector. The cells may then be plated on a 24-well plate and incubated at 37° C. for 3 days. The genomic DNA may be harvested on day 4 using the Beckman DNAdvance DNA extraction kit. The Fah amplicon may then be amplified and sequenced to measure the A->G editing efficiency.

Relative to FAH tgRNA without any synonymous mutations, mutations 1 and 2 are predicted to yield higher rewriting efficiencies. However mutation 3 is predicted to be too disruptive for efficient priming between priming region and the genomic DNA strand.

### Example 10: Improving Rewriting Performance by Disrupting Spacer: Priming Annealing Through Chemical Modifications

While synonymous mutations can disrupt priming: spacer annealing, they can also reduce annealing between priming region and the intended genomic sequence. Some chemical modifications such as N1-methyl deoxyadenosine, N1-methyl deoxyguanosine, N1-methyl adenosine or N1-methyl guanosine (PMID: 27478929) can be introduced in place of guanosine or adenosine. These chemical modifications are predicted to disrupt pairing between spacer and priming region but not with priming region and the intended genomic DNA sequence. Therefore, these modifications can be used to increase the overall rewriting performance.

To show that N1-methyl deoxyadenosine, N1-methyl deoxyguanosine, N1-methyl adenosine or N1-methyl guanosine can improve rewriting performance, the following RNA sequences may be chemically synthesized:

| Name | Priming sequence | Description |
|---|---|---|
| Unmodified | rGrUrUrCrArUrG | r = ribose |
| FAH-1 | rArGrGrArCrC | |
| priming | (SEQ ID NO: 22) | |
| Modified | rGrUrUrCrArUrG | x = N1-methyl |
| sequence 1 | rAxGrGrArCrC | guanosine |
| | (SEQ ID NO: 23) | |
| Modified | rGrUrUrCrArUrG | x = N1-methyl |
| sequence 2 | rAxGxGrArCrC | guanosine |
| | (SEQ ID NO: 24) | |
| Modified | rGrUrUrCxArUrG | x = N1-methyl |
| sequence 3 | rArGrGrArCrC | guanosine |
| | (SEQ ID NO: 25) | |

The tgRNA incorporating the chemical modifications may be nucleofected into HEK293T model cell line. 5 uM of tgRNA and 1 ug of ReWriter polypeptide mRNA (1 ug) may be mixed with 250,000 cells and nucleofected using the Lonza Nucleofector. The cells may then be plated on a 24-well plate and incubated at 37° C. for 3 days. The genomic DNA may be harvested on day 4 using the Beckman DNAdvance DNA extraction kit. The Fah amplicon may then be amplified and sequenced to measure the A->G editing efficiency. All three tested sequences are expected to show an increase in A→G conversion.

Other chemical modifications (e.g., as described herein) can be added to disrupt the priming sequence.

### Example 11: Improving Rewriting Performance by Disrupting Spacer: Priming Annealing Through Hairpins

To prevent annealing between the priming region and the spacer, hairpins of various lengths may be added to the priming region. It has previously been shown that addition of hairpins on the 5′ end of spacer can improve the specificity of Cas9 based editing by controlling the interaction between spacer and the genomic targets (PMID: 30988504). Here, we use a similar approach to disrupt annealing between priming region and the spacer region. Following sequences were designed with various stabilities to test this design. The free energies and structures were calculated using the RNAfold WebServer.

| | | Free Energy of |
|---|---|---|
| | Priming | hairpin-priming |
| Name | sequence | structure |
| Unmodifed FAH- | GUUCAUGAGGACC | NA (no hairpin) |
| 1 priming | (SEQ ID NO: 26) | |
| Hairpin 1 | GGUCCUCAUGAAGGA | −20.06 kcal/mol |
| | AAGUUCAUGAGGACC | |
| | (SEQ ID NO: 27) | |
| Hairpin 2 | GGACCUCAUCAAGGA | −8.60 kcal/mol |
| | AAGUUCAUGAGGACC | |
| | (SEQ ID NO: 28) | |
| Hairpin 3 | CCUCAUGAAGGAAA | −13.25 kcal/mol |
| | GUUCAUGAGGACC | |
| | (SEQ ID NO: 29) | |
| Hairpin 4 | CAUGAAGGAAAGUU | −5.46 kcal/mol |
| | CAUGAGGACC | |
| | (SEQ ID NO: 30) | |
| Hairpin 5 | AGGAAAGUUCAUGA | − 1.96 kcal/mol |
| | GGACC | |
| | (SEQ ID NO: 31) | |
| Hairpin 6 | GAAAGUUCAUGAGG | −1.95 kcal/mol |
| | ACC | |
| | (SEQ ID NO: 32) | |

The RNA designs may be chemically synthesized and then tested in HEK293T model cells. 5 uM of tgRNA and 1 ug of ReWriter polypeptide mRNA (1 ug) may be mixed with 250,000 cells and nucleofected using the Lonza Nucleofector. The cells may be plated on a 24-well plate and incubated at 37° C. for 3 days. The genomic DNA may be harvested on day 4 using the Beckman DNAdvance DNA extraction kit. The Fah amplicon may be amplified and sequenced to measure the A->G editing efficiency.

Hairpins with predicted stabilities −3 kcal/mol and −9 kcal/mol (hairpin 2 and 4) are expected to perform better than the unmodified FAH1 tgRNA. However, hairpins 3 and 1 with free energies of less than −10 kcal/mol are expected to perform worse than the unmodified FAH1 tgRNA. Lastly, hairpins 5 and 6 are expected to perform similarly to unmodified tgRNA.

Other RNA secondary structures such as aptazymes (PMID) hairpins containing photocleavable linkers (PMID) can also be used for the same purpose instead of hairpins described here.

### Example 12: Improving Rewriting Performance by Disrupting Secondary Structure Through Synonymous Mutations in the Template Region of tgRNA

Once the tgRNA is loaded into the Cas9, the scaffold and spacer region is shielded by the Cas9. However, the template and priming region remains unprotected and are free to form stable secondary structures that can hamper rewriting performance by preventing the RT from reverse transcribing the template region.

Synonymous mutations may be used to disrupt the secondary structure of FAH tgRNA for more efficient editing. The figure below shows the secondary structure of the template and priming region of FAH1 tgRNA:

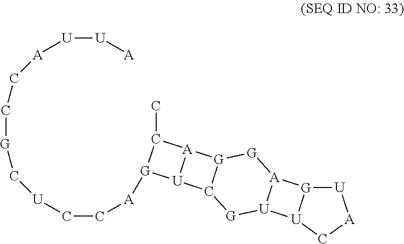

Exemplary templates for FAH1 tgRNA that contain synonymous mutations are listed below alongside the predicted free energy of the structure:

| | Template + | Free Energy of |
|---|---|---|
| | Priming | hairpin-priming |
| Name | sequence | structure |
| Unmodifed | AUUACCGCUCCAGU | −3.00 kcal/mol |
| FAH-1 | CGUUCAUGAGGACC | |
| | (SEQ ID NO: 34) | |
| Mutated | AUUACCGCUCCAAU | −2.40 kcal/mol |
| template 1 | CGUUCAUGAGGACC | |
| | (SEQ ID NO: 35) | |
| Mutated | AUUACCGCUCCAGU | −2.43 kcal/mol |
| template 2 | CGUUCAUUAGGACC | |
| | (SEQ ID NO: 36) | |
| Mutated | AUUACCGCUCCAGU | −2.61 kcal/mol |
| template 3 | CGUUCAUGAGAACC | |
| | (SEQ ID NO: 37) | |
| Mutated | AUUACCGCUCCAAU | −2.21 kcal/mol |
| template 4 | CGUUCAUUAGGACC | |
| | (SEQ ID NO: 38) | |
| Mutated | AUUACCGCUCCAGU | −2.57 kcal/mol |
| template 5 | CGUUCAUUAGAACC | |
| | (SEQ ID NO: 39) | |
| Mutated | AUUACCGCUCCAAU | −2.53 kcal/mol |
| template 6 | CGUUCAUUAGAACC | |
| | (SEQ ID NO: 40) | |

Template gRNAs containing the templates from the table above may chemically synthesized and nucleofected into HEK293T model cell lines. 5 uM of tgRNA and 1 ug of ReWriter polypeptide mRNA (1 ug) may be mixed with 250,000 cells and nucleofected using the Lonza Nucleofector. The cells then may be plated on a 24-well plate and incubated at 37° C. for 3 days. The genomic DNA may be harvested on day 4 using the Beckman DNAdvance DNA extraction kit. Fah amplicon may be PCR amplified using Fah specific primers and then sequenced. Rewriting efficiency then may be calculated by identifying percent of reads that contain the intended A->G edit.

All the designs with synonymous mutations are predicted to have less stable secondary structures relative to unmodified FAH1 tgRNA. All the designs are expected to result in higher efficiency than unmodified tgRNA.

### Example 13: Improving Rewriting Performance by Disrupting Secondary Structure Through Chemical Modifications in the Template Region of tgRNA

Similar to example 10, chemical modifications can also be used to disrupt the secondary structures within the template and priming region. Chemical modifications such as N1-methyl deoxyadenosine, N1-methyl deoxyguanosine, N1-methyl adenosine or N1-methyl guanosine (PMID: 27478929) will prevent stable secondary structures in the template and priming region without disrupting annealing with genomic sequence or integration into the genome.

To disrupt the secondary structure present in the template region of FAH tgRNA, introduced N1-methyl guanosine modification at the underlined position in the sequence below:

(SEQ ID NO: 41)
AUUACCGCUCCAGUCGUUCAUGAGGACC.

The chemically modified design is expected to disrupt the hairpin and increase rewriting performance.

### Example 14: Improving Template Guide RNA Stability by Circularization

In this example, the template guide RNA terminals are stabilized by connecting the 5′ and 3′ ends to form a circularized structure. Due to the lack of free ends, the circular template guide RNA is expected to have increased exonucleolytic stability compared to linear transcripts. Studies have shown that circular RNAs (circRNAs) accumulate in slow-dividing cells like neurons owing to their resistance to RNA degradation (Zhang et al. Cell Rep. 15: 611-624 (2016).

The circular tgRNAs (ctgRNAs) are synthesized by enzymatic ligation of the 5′ and 3′ ends of the linear transcript using either T4 DNA ligase and a DNA splint (Moore et al Science 256: 992-997 (2000). The linear tgRNAs are synthesized by conventional solid phase synthesis (SPS) using phosphoramidite chemistry. In cases where tgRNA are >150 nts, the synthesis of the linear tgRNAs is done in a stepwise manner utilizing enzymatic ligations at appropriate splice junctions on the transcript.

To assay designed circular tgRNAs (ctgRNAs), a Rewriter mRNA is co-transfected with template RNA into mouse hepatocytes derived from the mouse model Fah5981SB (PMID: 24681508). After three days, genomic DNA is extracted and analyzed for the efficiency of correct editing. In brief, primers flanking the Fah locus are used to amplify locus, and amplicons are analyzed by short-read sequencing (e.g. MiSeq).

The ctgRNAs with free ends are expected to increase exoribonuclease resistance of the Fah tgRNA resulting in higher rewriting efficiency as shown by higher fraction reads with correct A→G mutation.

### Example 15: Improving Secondary Structure of Template gRNA (tgRNA) by Annealing Nucleic Acids Oligos

In tgRNA in which the priming region is complementary to the spacer region, annealing between the two can impede loading into nCas9, thereby reducing nicking activity and decreasing overall system performance. The template region also may be prone to forming stable secondary structures that can prevent the RT from reading through the entire template, which results in lower intended genomic integrations.

Both problems can be reduced or prevented by including nucleic acid oligos that bind to the priming region. The oligos can interfere with annealing between the priming region and the spacer and can also block secondary structure formation by annealing to the sequences that participate in secondary structure formation. To serve these purposes, any nucleic acid oligos can be used e.g. UNA, PNA, FNA etc. Modified nucleic acids oligos such as LNA, PNA, FNA (2′-F RNA) are preferred in some embodiments because they are unlikely to trigger RNaseH-mediated degradation when exposed to the cellular environment, unlike DNA, are more nuclease resistant than RNA, and can bind RNA with higher affinity than DNA or RNA.

Another consideration for oligos design is the overall stability of duplex. The oligos should not be so stable that they stay bound to the tgRNA in the cell. For this reason, oligos of length 5-10 nt are preferred in certain embodiments.

In this example LNA oligos are shown to illustrate this concept. LNAs are highly nuclease resistant and bind RNA or DNA with higher affinity than RNA or DNA oligos. The Fah1 tgRNA was used to demonstrate the use of LNAs to block secondary structure formation and mis-annealing of priming and spacer regions. Fah1 tgRNA contains a single hairpin as predicted by RNAfold. Oligos are designed to anneal to the hairpin to prevent the formation of this hairpin. Since the hairpin is found in the priming sequence, oligos are also expected to disrupt priming and spacer interaction.

| | Design (Upper case = template + |
|---|---|
| | priming sequence and |
| | lowercase = annealed LNA |
| | sequence, underlined = |
| | sequence that form a hairpin, |
| Name | italics = priming) |
| Design 1 | 5′-AUUACCGCUCCAGUCGUUCAUGAGGACC-3′ |
| | (SEQ ID NO: 42) |
| | 3′-cagca-5′ |
| Design 2 | 5′-AUUACCGCUCCAGUCGUUCAUGAGGACC-3′ |
| | (SEQ ID NO: 44) |
| | 3′-cagcaa-5′ |
| Design 3 | 5′-AUUACCGCUCCAGUCGUUCAUGAGGACC-3′ |
| | (SEQ ID NO: 46) |
| | 3′-cagcaag-5′ |
| Design 4 | 5′-AUUACCGCUCCAGUCGUUCAUGAGGACC-3′ |
| | (SEQ ID NO: 48) |
| | 3′-cagcaagu-5′ |
| Design 5 | 5′-AUUACCGCUCCAGUCGUUCAUGAGGACC-3′ |
| | (SEQ ID NO: 50) |
| | 3′-cagcaagua-5′ |
| Design 6 | 5′-AUUACCGCUCCAGUCGUUCAUGAGGACC-3′ |
| | (SEQ ID NO: 52) |
| | 3′-cagcaaguac-5′ (SEQ ID NO: 53) |

Fah tgRNA and LNA oligos are chemically synthesized. The LNAs are annealed to Fah tgRNA in vitro by heating the nucleic acids to 90 C for 2 min and cooling to 25 C. The annealed complex is then nucleofected into HEK293T model cell line. 5 uM of tgRNA:LNA and 1 ug of ReWriter polypeptide mRNA (1 ug) is mixed with 250,000 cells and nucleofected using, e.g., the Lonza Nucleofector. The cells are then plated on a 24-well plate and incubated at 37° C. for 3 days. The genomic DNA is harvested on day 4 using the Beckman DNAdvance DNA extraction kit. Fah amplicon is PCR amplified using Fah specific primers and then sequenced. Rewriting efficiency is then calculated by identifying percent of reads that contain the intended A->G edit.

It is expected that the designed oligos will increase rewriting efficiency as measured by percent of reads with the intended A→G edit.

1-7. (canceled)

8. A system for modifying DNA comprising:
(a) a polypeptide or a nucleic acid encoding the polypeptide, wherein the polypeptide comprises a reverse transcriptase (RT) domain from a retrovirus and an endonuclease domain; and
(b) a template RNA (or DNA encoding the template RNA) comprising from 5′ to 3′ (i) a sequence that binds a target site in the DNA, (ii) a sequence that binds the polypeptide, (iii) a heterologous object sequence, (iv) a 3′ target homology domain, and (v) a region capable of hybridizing to any of (i), (ii), (iii), or (iv), or portions or combinations thereof.

9-10. (canceled)

11. A template RNA (or DNA encoding the template RNA) comprising from 5′ to 3′ (i) a sequence that binds a target site, (ii) a sequence that binds a polypeptide comprising a reverse transcriptase (RT) or RT domain, (iii) a heterologous object sequence, and (iv) a 3′ target homology domain,
wherein the heterologous object sequence comprises an alteration relative to a corresponding original sequence, wherein the alteration improves the speed, fidelity, or speed and fidelity of target-primed reverse transcription by a reverse transcriptase (RT),
or wherein the heterologous object sequence has one or both of the following characteristics:
i) does not comprise self-complementary sequences or if a self-complementary sequence is present, it has one, two, or all of the following characteristics:
(1) each self-complementary sequence is no more than 10, 9, 8, 7, 6, 5, 4, or 3 nucleotides in length,
(2) the self-complementary sequence forms a hairpin comprising arms of no longer than 10, 9, 8, 7, 6, 5, 4, or 3 nucleotides in length, or
(3) the self-complementary sequence comprises at least 1, 2, 3, 4, or 5 positions of non-complementarity with its partner sequence,
ii) does not comprise a repetitive sequence or if a repetitive sequence is present it is of no more than 12, 11, 10, 9, 8, 7, or 6 nucleotides in length.

12-18. (canceled)

19. A template RNA (or DNA encoding the template RNA) comprising from 5′ to 3′ (i) a sequence that binds a target site, (ii) a sequence that binds a polypeptide comprising a reverse transcriptase (RT) or RT domain, (iii) a heterologous object sequence, and (iv) a 3′ target homology domain,
wherein the template RNA further comprises an RT termination moiety situated between the heterologous object sequence and either (i) or (ii).

20-47. (canceled)

48. A lipid nanoparticle (LNP) comprising the system or template RNA, or DNA encoding the template RNA of claim 19.

49. (canceled)

50. A method for treating a subject having a disease or condition associated with a genetic defect, the method comprising:
administering to the subject the template RNA or DNA encoding the template RNA of claim 19,
thereby treating the subject having a disease or condition associated with a genetic defect.

51. A system comprising:
the template RNA of claim 19, and
a polypeptide or a nucleic acid encoding the polypeptide, wherein the polypeptide comprises a reverse transcriptase (RT) domain from a retrovirus and an endonuclease domain.

52. The template RNA of claim 19, wherein the RT termination moiety is situated between the heterologous object sequence and (i).

53. The template RNA of claim 19, wherein the RT termination moiety is situated between the heterologous object sequence and (ii).

54. The template RNA of claim 19, wherein the RT termination moiety comprises a non-nucleic acid molecule.

55. The template RNA of claim 19, wherein the RT termination moiety comprises a spacer.

56. The template RNA of claim 55, wherein the spacer is a C3 spacer or tri/hexa-ethylene glycol spacer.

57. The template RNA of claim 19, wherein the RT termination moiety comprises a trizole moiety.

58. The template RNA of claim 19, wherein the RT termination moiety comprises a streptavidin moiety.

59. The template RNA of claim 58, wherein the sequence that binds the polypeptide is attached to a first biotin moiety bound to the streptavidin moiety, and/or wherein the heterologous object sequence is attached to a second biotin moiety bound to the streptavidin moiety.

60. The template RNA of claim 19, wherein the RT termination moiety comprises a RT terminator sequence.

61. The template RNA of claim 60, wherein the RT terminator sequence comprises a sequence that adopts a secondary structure under physiological conditions.

62. The template RNA of claim 60, wherein the RT terminator sequence comprises a first self-complementary region and a second self-complementary region.

63. The template RNA of claim 60, wherein the RT terminator sequence adopts a secondary or tertiary structure comprising one or more hairpins.

64. The template RNA of claim 60, wherein the RT terminator sequence is situated no more than 1, 2, 3, 4, 5, 6, 7, 8, 9, 10, 11, 12, 13, 14, 15, 16, 17, 18, 19, 20, 30, 40, or 50 nucleotides from the heterologous object sequence.

65. The template RNA of claim 60, wherein the RT terminator sequence is situated directly adjacent to the heterologous object sequence.

66. The template RNA of claim 60, wherein the RT terminator sequence is situated no more than 1, 2, 3, 4, 5, 6, 7, 8, 9, 10, 11, 12, 13, 14, 15, 16, 17, 18, 19, 20, 30, 40, or 50 nucleotides from the sequence that binds the polypeptide.

67. The template RNA of claim 60, wherein the RT terminator sequence is situated directly adjacent to the sequence that binds the polypeptide.

68. The template RNA of claim 60, wherein the RT terminator sequence comprises:
(a) a sequence from the genome of a virus; or
(b) some or all of the HIV-1 central termination sequence (CTS).

69. The system of claim 51, wherein contacting a plurality of cells with the system produces fewer genomic modifications comprising template RNA sequence that is not the heterologous object sequence compared to contacting a similar plurality of cells with a similar system comprising a template RNA not comprising the RT terminator sequence.

70. The system of claim 51, wherein contacting a plurality of cells with the system produces fewer genomic modifications comprising (i) the sequence that binds the target site or (ii) the sequence that binds the polypeptide compared to contacting a similar plurality of cells with a similar system comprising a template RNA not comprising the RT terminator sequence.

71. The template RNA of claim 19, which comprises one or more chemically modified nucleotides.

72. The template RNA of claim 71, wherein the one or more chemically modified nucleotides comprise 1-methylguanosine, N6,N6-dimethyladenosine, or 3-methyluridine.


SEQUENCE LISTING (62 sequences)

SEQ ID NO: 1 - DNA, 16 bp, Adeno-associated virus 2
  source 1..16: mol_type = unassigned DNA
gcgcgctcgctcgctc

SEQ ID NO: 2 - skipped

SEQ ID NO: 3 - DNA, 76 bp, synthetic construct
  source 1..76: mol_type = other DNA; note = Description of Artificial Sequence: Synthetic oligonucleotide
gttttagagctagaaatagcaagttaaaataaggctagtccgttatcaacttgaaaaagtgggaccgagtcggtcc

SEQ ID NO: 4 - RNA, 110 nt, synthetic construct
  source 1..110: mol_type = other RNA; note = Description of Artificial Sequence: Synthetic polynucleotide
tgataataggctggagcctcggtggccatgcttcttgccccttgggcctccccccagcccctcctccccttcctgcacccgtacccccgtggtctttgaataaagtctga

SEQ ID NO: 5 - RNA, 221 nt, synthetic construct
  source 1..221: mol_type = other RNA; note = Description of Artificial Sequence: Synthetic polynucleotide
ggcggctgaggaagctgagggttttagagctagaaatagcaagttaaaataaggctagtccgttatcaacttgaaaaagtgggaccgagtcggtccagtccctcaagtccttccagcagcagcagcagcagcagcagcagcagcagcagcagcagcagcagcagcagcagcaacagccgccaccgccgccgccgccgccgccgcctcctcagcttcctcag

SEQ ID NO: 6 - DNA, 20 bp, synthetic construct
  source 1..20: mol_type = other DNA; note = Description of Artificial Sequence: Synthetic oligonucleotide
cgctgcaccgaccgtgagtt

SEQ ID NO: 7 - RNA, 221 nt, synthetic construct
  source 1..221: mol_type = other RNA; note = Description of Artificial Sequence: Synthetic polynucleotide
ggcggctgaggaagctgagggttttagagctagaaatagcaagttaaaataaggctagtccgttatcaacttgaaaaagtgggaccgagtcggtccagtccctcaagtccttccaacaacaacaacaacaacaacaacaacaacaacaacaacaacaacaacaacaacaacaacaaccacccccaccaccaccaccaccaccacccccacagcttcctcag

SEQ ID NO: 8 - DNA, 20 bp, synthetic construct
  source 1..20: mol_type = other DNA; note = Description of Artificial Sequence: Synthetic oligonucleotide
ggggccactagggacaggat

SEQ ID NO: 9 - RNA, 1065 nt, Homo sapiens
  source 1..1065: mol_type = unassigned RNA
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttga

SEQ ID NO: 10 - RNA, 1065 nt, synthetic construct
  source 1..1065: mol_type = other RNA; note = Description of Artificial Sequence: Synthetic polynucleotide
atgctgtttaatttgcggatcttgctcaacaatgccgcttttcgcaacggacacaacttcatggtgcgtaacttcagatgcggccagccgctgcaaaacaaagtccagctgaagggccgcgaccttttgaccctgaagaacttcactggcgaagagatcaagtacatgttgtggctgtctgccgatctgaaattccgtattaaacaaaagggtgagtacctgcctttgctgcagggaaagagtctgggtatgatttttgagaagcgctccaccaggacgcggctcagcaccgaaacaggattcgctctcctgggcgggcacccttgcttcctcaccacacaggacatccacctcggcgtgaacgagtccctgactgacactgcgcgcgtcctgtcttccatggccgatgctgtgctggctcgtgtgtacaaacagagcgacctggatactttggctaaagaggcctcaatccctattatcaatggtctttccgacctttatcatccgatccagatcttggctgactatctgactctgcaggaacactattcttccctgaaaggcctcaccttgtcatggattggcgacggcaataacatccttcacagcatcatgatgagcgccgcgaagttcgggatgcatctgcaagcggctacgcccaagggctacgagcccgatgccagtgtgaccaagctggctgagcagtatgccaaggaaaatggaacaaagcttctcctgaccaacgatccactcgaagcggctcacgggggtaacgtgctgattactgacacatggatctccatggggcaggaggaagagaagaaaaagcgcctgcaggcgtttcaggggtaccaggtgacaatgaagaccgctaaggtcgccgcttccgattggactttcttgcattgcctgcccagaaagcccgaagaggttgatgacgaggtgttttactcccctcgctccctggtgttccccgaggccgagaatcgcaaatggacaattatggctgtgatggtctctctcctgaccgactactcccctcagctccagaagccaaagttctaa

SEQ ID NO: 11 - RNA, 1065 nt, synthetic construct
  source 1..1065: mol_type = other RNA; note = Description of Artificial Sequence: Synthetic polynucleotide
atgctcttcaatctaagaatactactaaacaacgcagcattcagaaatggacacaactttatggtaagaaacttccgatgtggacaaccactacaaaacaaagtacaactaaaaggaagagacctattaacactcaaaaacttcacaggtgaagaaataaaatacatgctatggctatccgcagatttaaaattcagaataaaacaaaaaggagaatacctacctcttctacaaggaaaatcactaggaatgatattcgaaaaaagatcaacacgaacaagactaagcaccgaaaccggattcgcactactaggaggacatccctgcttccttacaactcaagacatccatttaggagtaaacgaatcactaaccgacacagcaagagtattaagctcaatggcagatgcagtacttgcaagagtatacaaacaatcagacctagacacactagcaaaagaagcatcaatcccaataataaacggactttcagacttataccatccaatacaaattttagcagactacttaaccttacaagaacactactcctcactgaaaggacttacactatcctggataggagacggaaacaacatcctacactcaataatgatgtcagcagcaaaattcggaatgcacctacaagcagccacaccaaaaggatacgaaccagatgcttcagtaacaaaactagcagaacaatacgcaaaagaaaacggaactaaactattattaacaaacgacccactcgaagcagcacacggaggaaacgtactcatcacagacacatggatatcaatgggacaagaagaagaaaaaaaaaaaagactacaagcattccaaggataccaagtaacaatgaaaacagcaaaagtcgcagcatccgactggacattcttacactgtctaccacgaaaaccagaagaagtcgacgacgaagtattctatagtccaagatcattagttttcccagaagcagaaaacagaaaatggaccataatggcagtaatggtatcactactaacagattactcaccccaactacaaaaaccaaaattttaa

SEQ ID NO: 12 - RNA, 221 nt, synthetic construct; identical to SEQ ID NO: 5
  source 1..221: mol_type = other RNA; note = Description of Artificial Sequence: Synthetic polynucleotide

SEQ ID NO: 13 - RNA, 246 nt, synthetic construct
  source 1..246: mol_type = other RNA; note = Description of Artificial Sequence: Synthetic polynucleotide
gaccctggaaaagctgatgagttttagagctagaaatagcaagttaaaataaggctagtccgttatcaacttgaaaaagtgggaccgagtcggtccctgaggaagctgaggaggcggcggcggcggcggcggcggtggcggctgttgctgctgctgctgctgctgctgctgctgctgctgctgctgctgctgctgctgctgctggaaggacttgagggactcgaaggccttcatcagcttttccag

SEQ ID NO: 14 - RNA, 124 nt, synthetic construct
  source 1..124: mol_type = other RNA; note = Description of Artificial Sequence: Synthetic polynucleotide
ggatggtcctcatgaacgacgttttagagctagaaatagcaagttaaaataaggctagtccgttatcaacttgaaaaagtggcaccgagtcggtgcattaccgctccagtcgttcatgaggacc

SEQ ID NO: 15 - RNA, 124 nt, synthetic construct; identical to SEQ ID NO: 14
  source 1..124: mol_type = other RNA; note = Description of Artificial Sequence: Synthetic polynucleotide
  misc_difference 124: note = Cytidine attached to 3'-Biotin, 3'-Biotin-TEG or Cholesterol-TEG

SEQ ID NO: 16 - RNA, 141 nt, synthetic construct
  source 1..141: mol_type = other RNA; note = Description of Artificial Sequence: Synthetic polynucleotide
ggatggtcctcatgaacgacgttttagagctagaaatagcaagttaaaataaggctagtccgttatcaacttgaaaaagtggcaccgagtcggtgcattaccgctccagtcgttcatgaggaccacatcaggtggtggtgg

SEQ ID NO: 17 - RNA, 124 nt, synthetic construct; identical to SEQ ID NO: 14
  source 1..124: mol_type = other RNA; note = Description of Artificial Sequence: Synthetic polynucleotide

SEQ ID NO: 18 - RNA, 13 nt, unidentified
  source 1..13: mol_type = unassigned RNA; note = Description of Unknown: FAH-1 sequence
gttcatgaggacc

SEQ ID NO: 19 - RNA, 13 nt, synthetic construct
  source 1..13: mol_type = other RNA; note = Description of Artificial Sequence: Synthetic oligonucleotide
gttcatgagaacc

SEQ ID NO: 20 - RNA, 13 nt, synthetic construct
  source 1..13: mol_type = other RNA; note = Description of Artificial Sequence: Synthetic oligonucleotide
gttcattagaacc

SEQ ID NO: 21 - RNA, 13 nt, synthetic construct
  source 1..13: mol_type = other RNA; note = Description of Artificial Sequence: Synthetic oligonucleotide
gttcatttaaacc

SEQ ID NO: 22 - RNA, 13 nt, unidentified
  source 1..13: mol_type = unassigned RNA; note = Description of Unknown: FAH-1 sequence
gttcatgaggacc

SEQ ID NO: 23 - RNA, 14 nt, synthetic construct
  source 1..14: mol_type = other RNA; note = Description of Artificial Sequence: Synthetic oligonucleotide
  modified_base 9: mod_base = OTHER; note = N1-methyl guanosine
gttcatgagggacc

SEQ ID NO: 24 - RNA, 15 nt, synthetic construct
  source 1..15: mol_type = other RNA; note = Description of Artificial Sequence: Synthetic oligonucleotide
  modified_base 9: mod_base = OTHER; note = N1-methyl guanosine
  modified_base 11: mod_base = OTHER; note = N1-methyl guanosine
gttcatgaggggacc

SEQ ID NO: 25 - RNA, 14 nt, synthetic construct
  source 1..14: mol_type = other RNA; note = Description of Artificial Sequence: Synthetic oligonucleotide
  modified_base 5: mod_base = OTHER; note = N1-methyl adenosine
gttcaatgaggacc

SEQ ID NO: 26 - RNA, 13 nt, unidentified
  source 1..13: mol_type = unassigned RNA; note = Description of Unknown: FAH-1 sequence
gttcatgaggacc

SEQ ID NO: 27 - RNA, 30 nt, synthetic construct
  source 1..30: mol_type = other RNA; note = Description of Artificial Sequence: Synthetic oligonucleotide
ggtcctcatgaaggaaagttcatgaggacc

SEQ ID NO: 28 - RNA, 30 nt, synthetic construct
  source 1..30: mol_type = other RNA; note = Description of Artificial Sequence: Synthetic oligonucleotide
ggacctcatcaaggaaagttcatgaggacc

SEQ ID NO: 29 - RNA, 27 nt, synthetic construct
  source 1..27: mol_type = other RNA; note = Description of Artificial Sequence: Synthetic oligonucleotide
cctcatgaaggaaagttcatgaggacc

SEQ ID NO: 30 - RNA, 24 nt, synthetic construct
  source 1..24: mol_type = other RNA; note = Description of Artificial Sequence: Synthetic oligonucleotide
catgaaggaaagttcatgaggacc

SEQ ID NO: 31 - RNA, 19 nt, synthetic construct
  source 1..19: mol_type = other RNA; note = Description of Artificial Sequence: Synthetic oligonucleotide
aggaaagttcatgaggacc

SEQ ID NO: 32 - RNA, 17 nt, synthetic construct
  source 1..17: mol_type = other RNA; note = Description of Artificial Sequence: Synthetic oligonucleotide
gaaagttcatgaggacc

SEQ ID NO: 33 - RNA, 28 nt, synthetic construct
  source 1..28: mol_type = other RNA; note = Description of Artificial Sequence: Synthetic oligonucleotide
attaccgctccagtcgttcatgaggacc

SEQ ID NO: 34 - RNA, 28 nt, unidentified
  source 1..28: mol_type = unassigned RNA; note = Description of Unknown: FAH-1 sequence
attaccgctccagtcgttcatgaggacc

SEQ ID NO: 35 - RNA, 28 nt, synthetic construct
  source 1..28: mol_type = other RNA; note = Description of Artificial Sequence: Synthetic oligonucleotide
attaccgctccaatcgttcatgaggacc

SEQ ID NO: 36 - RNA, 28 nt, synthetic construct
  source 1..28: mol_type = other RNA; note = Description of Artificial Sequence: Synthetic oligonucleotide
attaccgctccagtcgttcattaggacc

SEQ ID NO: 37 - RNA, 28 nt, synthetic construct
  source 1..28: mol_type = other RNA; note = Description of Artificial Sequence: Synthetic oligonucleotide
attaccgctccagtcgttcatgagaacc

SEQ ID NO: 38 - RNA, 28 nt, synthetic construct
  source 1..28: mol_type = other RNA; note = Description of Artificial Sequence: Synthetic oligonucleotide
attaccgctccaatcgttcattaggacc

SEQ ID NO: 39 - RNA, 28 nt, synthetic construct
  source 1..28: mol_type = other RNA; note = Description of Artificial Sequence: Synthetic oligonucleotide
attaccgctccagtcgttcattagaacc

SEQ ID NO: 40 - RNA, 28 nt, synthetic construct
  source 1..28: mol_type = other RNA; note = Description of Artificial Sequence: Synthetic oligonucleotide
attaccgctccaatcgttcattagaacc

SEQ ID NO: 41 - RNA, 28 nt, synthetic construct
  source 1..28: mol_type = other RNA; note = Description of Artificial Sequence: Synthetic oligonucleotide
  modified_base 13: mod_base = OTHER; note = N1-methyl guanosine
attaccgctccagtcgttcatgaggacc

SEQ ID NO: 42 - RNA, 28 nt, synthetic construct
  source 1..28: mol_type = other RNA; note = Description of Artificial Sequence: Synthetic oligonucleotide
attaccgctccagtcgttcatgaggacc

SEQ ID NO: 43 - skipped

SEQ ID NO: 44 - RNA, 28 nt, synthetic construct
  source 1..28: mol_type = other RNA; note = Description of Artificial Sequence: Synthetic oligonucleotide
attaccgctccagtcgttcatgaggacc

SEQ ID NO: 45 - skipped

SEQ ID NO: 46 - RNA, 28 nt, synthetic construct
  source 1..28: mol_type = other RNA; note = Description of Artificial Sequence: Synthetic oligonucleotide
attaccgctccagtcgttcatgaggacc

SEQ ID NO: 47 - skipped

SEQ ID NO: 48 - RNA, 28 nt, synthetic construct
  source 1..28: mol_type = other RNA; note = Description of Artificial Sequence: Synthetic oligonucleotide
attaccgctccagtcgttcatgaggacc

SEQ ID NO: 49 - skipped

SEQ ID NO: 50 - RNA, 28 nt, synthetic construct
  source 1..28: mol_type = other RNA; note = Description of Artificial Sequence: Synthetic oligonucleotide
attaccgctccagtcgttcatgaggacc

SEQ ID NO: 51 - skipped

SEQ ID NO: 52 - RNA, 28 nt, synthetic construct
  source 1..28: mol_type = other RNA; note = Description of Artificial Sequence: Synthetic oligonucleotide
attaccgctccagtcgttcatgaggacc

SEQ ID NO: 53 - RNA, 10 nt, synthetic construct
  source 1..10: mol_type = other RNA; note = Description of Artificial Sequence: Synthetic oligonucleotide
catgaacgac

SEQ ID NO: 54 - DNA, 26 bp, Homo sapiens
  source 1..26: mol_type = unassigned DNA
ctgacccttctctctctcccccagag

SEQ ID NO: 55 - DNA, 15 bp, Homo sapiens
  source 1..15: mol_type = unassigned DNA
tttctctcccacaag

SEQ ID NO: 56 - RNA, 47 nt, synthetic construct
  source 1..47: mol_type = other RNA; note = Description of Artificial Sequence: Synthetic oligonucleotide
gggaaataagagagaaaagaagagtaagaagaaatataagagccacc

SEQ ID NO: 57 - RNA, 221 nt, synthetic construct
  source 1..221: mol_type = other RNA; note = Description of Artificial Sequence: Synthetic polynucleotide
ggcggctgaggaagctgagggtgttttagagctagaaatagcaagttaaaataaggctagtccgttatcaacttgaaaaagtgggaccgagtcggtccagtccctcaagtccttccagcagcagcagcagcagcagcagcagcagcagcagcagcagcagcagcagcagcagcaacagccgccaccgccgccgccgccgccgccgccctcagcttcctcag

SEQ ID NO: 58 - RNA, 221 nt, synthetic construct; identical to SEQ ID NO: 7
  source 1..221: mol_type = other RNA; note = Description of Artificial Sequence: Synthetic polynucleotide

SEQ ID NO: 59 - RNA, 100 nt, synthetic construct
  source 1..100: mol_type = other RNA; note = Description of Artificial Sequence: Synthetic polynucleotide
  misc_difference 1: note = modified_base - a, c, u, g, unknown or other
  misc_difference 2: note = modified_base - a, c, u, g, unknown or other
  misc_difference 3: note = modified_base - a, c, u, g, unknown or other
  misc_difference 4: note = modified_base - a, c, u, g, unknown or other
  misc_difference 5: note = modified_base - a, c, u, g, unknown or other
  misc_difference 6: note = modified_base - a, c, u, g, unknown or other
  misc_difference 7: note = modified_base - a, c, u, g, unknown or other
  misc_difference 8: note = modified_base - a, c, u, g, unknown or other
  misc_difference 9: note = modified_base - a, c, u, g, unknown or other
  misc_difference 10: note = modified_base - a, c, u, g, unknown or other
  misc_difference 11: note = modified_base - a, c, u, g, unknown or other
  misc_difference 12: note = modified_base - a, c, u, g, unknown or other
  misc_difference 13: note = modified_base - a, c, u, g, unknown or other
  misc_difference 14: note = modified_base - a, c, u, g, unknown or other
  misc_difference 15: note = modified_base - a, c, u, g, unknown or other
  misc_difference 16: note = modified_base - a, c, u, g, unknown or other
  misc_difference 17: note = modified_base - a, c, u, g, unknown or other
  misc_difference 18: note = modified_base - a, c, u, g, unknown or other
  misc_difference 19: note = modified_base - a, c, u, g, unknown or other
  misc_difference 20: note = modified_base - a, c, u, g, unknown or other
nnnnnnnnnnnnnnnnnnnngttttagagctagaaatagcaagttaaaataaggctagtccgttatcaacttgaaaaagtggcaccgagtcggtgctttt

SEQ ID NO: 60 - RNA, 101 nt, synthetic construct
  source 1..101: mol_type = other RNA; note = Description of Artificial Sequence: Synthetic polynucleotide
gggcgaggagctgttcaccggttttagagctagaaatagcaagttaaaataaggctagtccgttatcaacttgaaaaagtggcaccgagtcggtgcttttt

SEQ ID NO: 61 - RNA, 36 nt, synthetic construct
  source 1..36: mol_type = other RNA; note = Description of Artificial Sequence: Synthetic oligonucleotide
ggtgagctcttatttgcgtagttttagagctatgct

SEQ ID NO: 62 - RNA, 67 nt, synthetic construct
  source 1..67: mol_type = other RNA; note = Description of Artificial Sequence: Synthetic oligonucleotide
agcatagcaagttaaaataaggctagtccgttatcaacttgaaaaagtggcaccgagtcggtgcttt